(19) United States
(12) Patent Application Publication
(10) Pub. No.: US 2024/0101716 A1
(43) Pub. Date: Mar. 28, 2024

(54) BISPECIFIC ANTIBODIES TARGETING SIRPa AND PD-L1

(71) Applicant: La Nova Medicines Development Co., Ltd., Shanghai (CN)

(72) Inventors: Runsheng LI, Shanghai (CN); Ying Qin ZANG, Shanghai (CN)

(21) Appl. No.: 18/267,397

(22) PCT Filed: Dec. 17, 2021
(86) PCT No.: PCT/CN2021/139115

§ 371 Date: Jun. 14, 2023

(30) Foreign Application Priority Data
Dec. 18, 2020 (WO) PCT/CN2020/137579

(51) Int. Cl. C07K 16/46; A61P 35/00

(52) U.S. Cl. CPC C07K 16/468; A61P 35/00; C07K 2317/31; C07K 2317/52; C07K 2317/55; C07K 2317/565; C07K 2317/569

(57) ABSTRACT

Provided are antibodies and fragments having binding specificity to the signal regulatory protein alpha (SIRPα) protein and to the programmed death-ligand 1 (PD-L1) protein. The antibodies and fragments can bring peripheral macrophages to PD-L1+ tumor site, thereby enhancing tumor treatment.

### BACKGROUND

Signal regulatory protein alpha (SIRPα) is a member of the signal-regulatory-protein (SIRP) family, and also belongs to the immunoglobulin superfamily. SIRPα recognizes CD47, an anti-phagocytic signal that distinguishes live cells from dying cells. The extracellular domain of SIRPα binds to CD47 and transmits intracellular signals through its cytoplasmic domain. CD47-binding is mediated through the NH2-terminal V-like domain of SIRPα. The cytoplasmic region contains four ITIMs that become phosphorylated after binding of ligand. The phosphorylation mediates activation of tyrosine kinase SHP2. SIRPα also binds phosphatase SHP1, adaptor protein SCAP2 and FYN-binding protein. Recruitment of SHP phosphatases to the membrane leads to the inhibition of myosin accumulation at the cell surface and results in the inhibition of phagocytosis.

Cancer cells highly express CD47 that activates SIRPα and inhibits macrophage-mediated destruction. It has been shown that high-affinity variants of SIRPα that antagonized CD47 on cancer cells increased phagocytosis of cancer cells. Anti-SIRPα antibodies have also been shown to help macrophages to reduce cancer growth and metastasis, alone and in synergy with other cancer treatments.

Programmed death-ligand 1 (PD-L1), also known as cluster of differentiation 274 (CD274) or B7 homolog 1 (B7-H1), is a 40 kDa type 1 transmembrane protein believed to play a major role in suppressing the immune system during particular events such as pregnancy, tissue allografts, autoimmune disease and other disease states such as hepatitis. The binding of PD-L1 to PD-1 or B7.1 transmits an inhibitory signal which reduces the proliferation of CD8+ T cells at the lymph nodes and supplementary to that PD-1 is also able to control the accumulation of foreign antigen specific T cells in the lymph nodes through apoptosis which is further mediated by a lower regulation of the gene Bcl-2.

Bispecific antibodies targeting both the SIRPα and PD-L1 proteins have been proposed, but development of bispecific antibodies with good stability and activity has been proven to be challenging.

### SUMMARY

The present disclosure, in some embodiments, discloses antibodies targeting both SIRPα and PD-L1 to enhance both T cell function and macrophage phagocytosis for treating cancers. By virtue of the specificity to PD-L1, the antibodies cam bring peripheral M1 macrophages to the tumor site that is positive of PD-L1. The anti-SIRPα specificity can block CD47/SIRPα interaction thereby enhancing the engulfment of tumor cells by macrophages. It can also enable dendritic cells to process and present tumor antigens, leading to priming and boosting of tumor-specific CD8+effector T cells.

Dual targeting of these antibodies at both the innate and the adaptive immune checkpoints, therefore, can maximize anti-tumor therapeutic effect and elicit more durable responses. Moreover, these antibodies can have better safety profiles as compared to anti-CD47 antibodies.

In accordance with one embodiment of the present disclosure, provided is an antibody comprising an anti-signal regulatory protein alpha (SIRPα) unit and an anti-programmed death-ligand 1 (PD-L1) unit, wherein the anti-SIRPα unit comprises an Fab fragment having binding specificity to a human SIRPα protein, and the anti-PD-L1 unit comprises a single-domain antibody (sdAb) having binding specificity to a human PD-L1 protein.

In some embodiments, the antibody further comprises an Fc fragment. In some embodiments, the sdAb is fused to the heavy chain of the Fab fragment. In some embodiments, the sdAb is fused to the light chain of the Fab fragment. In some embodiments, the sdAb is fused to the C-terminus of the heavy chain. In some embodiments, the sdAb is fused to the N-terminus of the heavy chain.

Specific sequences for the anti-SIRPα unit and the anti-PD-L1 unit are also disclosed herein.

Also provided, in some embodiments, are compositions comprising the antibody or fragment thereof and a pharmaceutically acceptable carrier. In some embodiments, the composition further comprises a second antibody having specificity to a tumor antigen. In some embodiments, the second antibody is a tumor-opsonizing antibody.

Methods and uses for the treatment of diseases and conditions are also provided. In one embodiment, provided is a method of treating cancer in a patient in need thereof, comprising administering to the patient the antibody or fragment thereof of the present disclosure.

### BRIEF DESCRIPTION OF THE DRAWINGS

FIG. 1 shows cross-binding to SIRPα v1 and v2 by the antibodies.

FIG. 2 shows binding affinity of the antibody against SIRPα v1.

FIG. 3 shows competition of SIRPα interaction with CD47 by the antibodies.

FIG. 4 shows induction of macrophage mediated phagocytosis by the antibodies.

FIG. 5 shows increase of macrophage mediated phagocytosis of tumor cells by the antibody treatments.

FIG. 6A-D illustrate four different formats of the bispecific antibodies.

FIG. 7 shows that the bispecific antibodies had high affinity for PD-L1 expressed on cells.

FIG. 8 shows that the bispecific antibodies had high affinity for SIRPα expressed on cells.

FIG. 9 shows that the bispecific antibodies were effective in blocking PD-1 and PD-L1 interactions.

FIG. 10 shows that the bispecific antibodies were effective in blocking CD47 and SIRPα interactions.

FIG. 11 shows that the bispecific antibodies had potent activities in inducing phagocytosis.

### DETAILED DESCRIPTION

### Definitions

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

The term antibody encompasses various broad classes of polypeptides that can be distinguished biochemically. Those skilled in the art will appreciate that heavy chains are classified as gamma, mu, alpha, delta, or epsilon (γ, μ, α, δ, ϵ) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

The term “heavy chain-only antibody” or “HCAb” refers to a functional antibody, which comprises heavy chains, but lacks the light chains usually found in 4-chain antibodies. Camelid animals (such as camels, llamas, or alpacas) are known to produce HCAbs.

The term “single-domain antibody” or “sdAb” refers to a single antigen-binding polypeptide having three complementary determining regions (CDRs). The sdAb alone is capable of binding to the antigen without pairing with a corresponding CDR-containing polypeptide. In some cases, single-domain antibodies are engineered from camelid HCAbs, and their heavy chain variable domains are referred herein as “V_{H}Hs” (Variable domain of the heavy chain of the Heavy chain antibody). Some V_{H}Hs can also be known as nanobodies. Camelid sdAb is one of the smallest known antigen-binding antibody fragments (see, e.g., Hamers-Casterman et al., Nature 363:446-8 (1993); Greenberg et al., Nature 374:168-73 (1995); Hassanzadeh-Ghassabeh et al., Nanomedicine (Lond), 8:1013-26 (2013)). A basic V_{H}H has the following structure from the N-terminus to the C-terminus: FR1-CDR1-FR2-CDR2-FR3-CDR3-FR4, in which FR1 to FR4 refer to framework regions 1 to 4, respectively, and in which CDR1 to CDR3 refer to the complementarity determining regions 1 to 3.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### Bispecific Antibodies

PD-L1 is a critical “don't find me” signal to the adaptive immune system. CD47/SIRPα transmits an anti-phagocytic signal, known as the “don't eat me” signal, to the innate immune system. It is contemplated that dual targeting both innate and adaptive immune checkpoints can help maximize anti-tumor therapeutic effect and elicit more durable responses.

The present disclosure provides anti-SIRPα antibodies and fragments that have high affinity to both variants v1 and v2. Variant 1 (hSIRPαV1) is the dominant variant among Europeans, Africans, Ad mixed Americans, and South Asians. Variant 2 (hSIRPαV2) is the dominant variant among East Asians. Sequences of hSIRPαV1 and hSIRPαV2 differ within the extracellular Ig-like V-like (IgV) domain. The ability of the instantly disclosed antibodies and fragments to recognize both variants enables them to be effective among the widest patient population.

The present disclosure also describes single-domain antibodies (sdAb) specifically recognizing PD-L1, as well as heavy chain-only antibody (HCAb). Single-chain antibodies (sdAbs) are different from conventional 4-chain antibodies by having a single monomeric antibody variable domain, such as heavy chain variable domain (V_{H}H), which can exhibit high affinity to an antigen without the aid of a light chain.

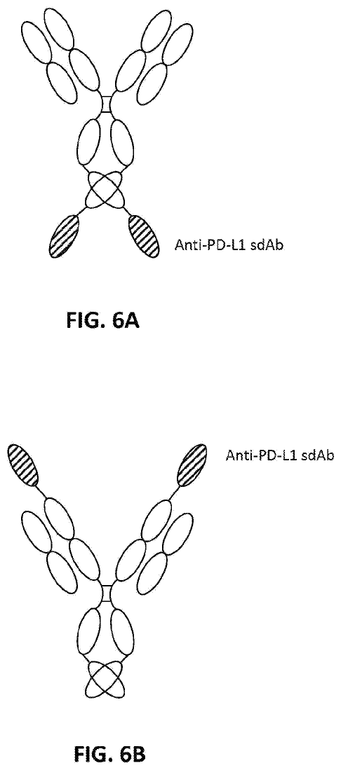

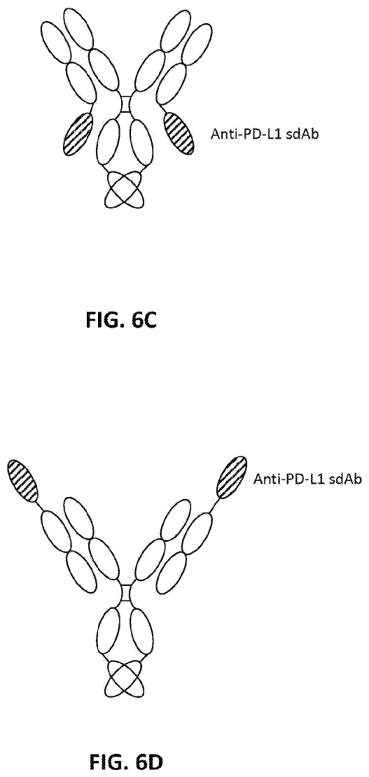

The anti-SIRPα antibodies and fragments and the anti-PD-L1 sdAbs, in some embodiments, are fused to form a bispecific antibody. Four bispecific antibody formats were tested in this disclosure, which are illustrated in FIG. 6A-D. In the first format, the sdAb is fused to the C-terminal end of the heavy chain of the Fab fragment. In the second format, the sdAb is fused to the N-terminal end of the heavy chain of the Fab fragment. In the third format, the sdAb is fused to the C-terminal end of the light chain of the Fab fragment. In the fourth format, the sdAb is fused to the N-terminal end of the light chain of the Fab fragment.

In some embodiments, the Fab fragment can further include a Fc fragment, as illustrated in the formats of FIG. 6. In a preferred embodiment, the format of FIG. 6A is used, in which an anti-PD-L1 sdAb is fused, through a (G4S)n linker, to the C-terminus of the Fc fragment of the full anti-SIRPα antibody.

In accordance with one embodiment of the present disclosure, provided is an antibody comprising an anti-signal regulatory protein alpha (SIRPα) unit and an anti-programmed death-ligand 1 (PD-L1) unit, wherein the anti-SIRPα unit comprises an Fab fragment having binding specificity to a human SIRPα protein, and the anti-PD-L1 unit comprises a single-domain antibody (sdAb) having binding specificity to a human PD-L1 protein.

In some embodiments, the antibody further comprises an Fc fragment. In some embodiments, the sdAb is fused to the heavy chain of the Fab fragment. In some embodiments, the sdAb is fused to the light chain of the Fab fragment. In some embodiments, the sdAb is fused to the C-terminus of the heavy chain. In some embodiments, the sdAb is fused to the N-terminus of the heavy chain.

Examples of anti-PD-L1 sdAbs and anti-SIRPα antibodies are also described herein.

### Anti-SIRPα Unit

In accordance with one embodiment of the present disclosure, therefore, provided are antibodies and antigen-binding fragments thereof that are able to bind to both variants 1 and 2 of SIRPα. Example antibodies include those murine ones listed in Table 1, as well as humanized ones of Tables 2-8. Also included are those that include the same CDRs as illustrated herein. In some embodiments, the disclosed antibodies and fragments include those that bind to the same epitope as those illustrated here, and those that compete with the instantly disclosed in binding to SIRPα.

In accordance with one embodiment of the present disclosure, provided is an antibody or fragment thereof that includes the heavy chain and light chain variable domains with the CDR regions disclosed herein, as well as their biological equivalents.

In one embodiment, the CDRs are those of 248G3F6, as exemplified in Tables 2B and 2D. In one embodiment, the CDRH1 comprises the amino acid sequence of SEQ ID NO: 15 or a variant thereof having one, two, or three deletions, additions, substitutions or the combinations thereof, the CDRH2 comprises the amino acid sequence of SEQ ID NO: 16, 21 or 22 or a variant thereof having one, two, or three deletions, additions, substitutions or the combinations thereof, the CDRH3 comprises the amino acid sequence of SEQ ID NO: 17 or a variant thereof having one, two, or three deletions, additions, substitutions or the combinations thereof, the CDRL1 comprises the amino acid sequence of SEQ ID NO: 18 or a variant thereof having one, two, or three deletions, additions, substitutions or the combinations thereof, the CDRL2 comprises the amino acid sequence of SEQ ID NO: 19 or a variant thereof having one, two, or three deletions, additions, substitutions or the combinations thereof, and the CDRL3 comprises the amino acid sequence of SEQ ID NO: 20 or a variant thereof having one, two, or three deletions, additions, substitutions or the combinations thereof.

One embodiment provides an antibody or fragment thereof having binding specificity to a wild-type human signal regulatory protein alpha (SIRPα) protein, wherein the antibody or fragment thereof comprises a heavy chain variable region comprising heavy chain complementarity determining regions CDRH1, CDRH2, and CDRH3 and a light chain variable region light chain comprising complementarity determining regions CDRL1, CDRL2, and CDRL3, and wherein the CDRH1 comprises the amino acid sequence of SEQ ID NO: 15, the CDRH2 comprises the amino acid sequence of SEQ ID NO: 16, the CDRH3 comprises the amino acid sequence of SEQ ID NO: 17, the CDRL1 comprises the amino acid sequence of SEQ ID NO: 18, the CDRL2 comprises the amino acid sequence of SEQ ID NO: 19, and the CDRL3 comprises the amino acid sequence of SEQ ID NO: 20.

One embodiment provides an antibody or fragment thereof having binding specificity to a wild-type human signal regulatory protein alpha (SIRPα) protein, wherein the antibody or fragment thereof comprises a heavy chain variable region comprising heavy chain complementarity determining regions CDRH1, CDRH2, and CDRH3 and a light chain variable region light chain comprising complementarity determining regions CDRL1, CDRL2, and CDRL3, and wherein the CDRH1 comprises the amino acid sequence of SEQ ID NO: 15, the CDRH2 comprises the amino acid sequence of SEQ ID NO: 21, the CDRH3 comprises the amino acid sequence of SEQ ID NO: 17, the CDRL1 comprises the amino acid sequence of SEQ ID NO: 18, the CDRL2 comprises the amino acid sequence of SEQ ID NO: 19, and the CDRL3 comprises the amino acid sequence of SEQ ID NO: 20.

One embodiment provides an antibody or fragment thereof having binding specificity to a wild-type human signal regulatory protein alpha (SIRPα) protein, wherein the antibody or fragment thereof comprises a heavy chain variable region comprising heavy chain complementarity determining regions CDRH1, CDRH2, and CDRH3 and a light chain variable region light chain comprising complementarity determining regions CDRL1, CDRL2, and CDRL3, and wherein the CDRH1 comprises the amino acid sequence of SEQ ID NO: 15, the CDRH2 comprises the amino acid sequence of SEQ ID NO: 22, the CDRH3 comprises the amino acid sequence of SEQ ID NO: 17, the CDRL1 comprises the amino acid sequence of SEQ ID NO: 18, the CDRL2 comprises the amino acid sequence of SEQ ID NO: 19, and the CDRL3 comprises the amino acid sequence of SEQ ID NO: 20.

In some embodiments, the heavy chain variable region comprises an amino acid sequence selected from the group consisting of SEQ ID NO:1 and 23-27, or a peptide having at least 90% sequence identity to an amino acid sequence selected from the group consisting of SEQ ID NO:1 and 23-27.

In some embodiments, the light chain variable region comprises an amino acid sequence selected from the group consisting of SEQ ID NO:2 and 28-29, or a peptide having at least 90% sequence identity to an amino acid sequence selected from the group consisting of SEQ ID NO:2 and 28-29.

In some embodiments, the heavy chain variable region comprises the amino acid sequence of SEQ ID NO:27 and the light chain variable region comprises the amino acid sequence of SEQ ID NO:29.

In one embodiment, the CDRs are those of 300A6A6, as exemplified in Tables 3B and 3D. In one embodiment, the CDRH1 comprises the amino acid sequence of SEQ ID NO: 30 or a variant thereof having one, two, or three deletions, additions, substitutions or the combinations thereof, the CDRH2 comprises the amino acid sequence of SEQ ID NO: 31, 36, 37 or 38 or a variant thereof having one, two, or three deletions, additions, substitutions or the combinations thereof, the CDRH3 comprises the amino acid sequence of SEQ ID NO: 32 or a variant thereof having one, two, or three deletions, additions, substitutions or the combinations thereof, the CDRL1 comprises the amino acid sequence of SEQ ID NO: 33 or a variant thereof having one, two, or three deletions, additions, substitutions or the combinations thereof, the CDRL2 comprises the amino acid sequence of SEQ ID NO: 34 or a variant thereof having one, two, or three deletions, additions, substitutions or the combinations thereof, and the CDRL3 comprises the amino acid sequence of SEQ ID NO: 35 or a variant thereof having one, two, or three deletions, additions, substitutions or the combinations thereof.

In one embodiment, provided is an antibody or fragment thereof having binding specificity to a wild-type human signal regulatory protein alpha (SIRPα) protein, wherein the antibody or fragment thereof comprises a heavy chain variable region comprising heavy chain complementarity determining regions CDRH1, CDRH2, and CDRH3 and a light chain variable region light chain comprising complementarity determining regions CDRL1, CDRL2, and CDRL3, and wherein the CDRH1 comprises the amino acid sequence of SEQ ID NO: 30, the CDRH2 comprises the amino acid sequence of SEQ ID NO: 31, the CDRH3 comprises the amino acid sequence of SEQ ID NO: 32, the CDRL1 comprises the amino acid sequence of SEQ ID NO: 33, the CDRL2 comprises the amino acid sequence of SEQ ID NO: 34, and the CDRL3 comprises the amino acid sequence of SEQ ID NO: 35.

In one embodiment, provided is an antibody or fragment thereof having binding specificity to a wild-type human signal regulatory protein alpha (SIRPα) protein, wherein the antibody or fragment thereof comprises a heavy chain variable region comprising heavy chain complementarity determining regions CDRH1, CDRH2, and CDRH3 and a light chain variable region light chain comprising complementarity determining regions CDRL1, CDRL2, and CDRL3, and wherein the CDRH1 comprises the amino acid sequence of SEQ ID NO: 30, the CDRH2 comprises the amino acid sequence of SEQ ID NO: 36, the CDRH3 comprises the amino acid sequence of SEQ ID NO: 32, the CDRL1 comprises the amino acid sequence of SEQ ID NO: 33, the CDRL2 comprises the amino acid sequence of SEQ ID NO: 34, and the CDRL3 comprises the amino acid sequence of SEQ ID NO: 35.

In one embodiment, provided is an antibody or fragment thereof having binding specificity to a wild-type human signal regulatory protein alpha (SIRPα) protein, wherein the antibody or fragment thereof comprises a heavy chain variable region comprising heavy chain complementarity determining regions CDRH1, CDRH2, and CDRH3 and a light chain variable region light chain comprising complementarity determining regions CDRL1, CDRL2, and CDRL3, and wherein the CDRH1 comprises the amino acid sequence of SEQ ID NO: 30, the CDRH2 comprises the amino acid sequence of SEQ ID NO: 37, the CDRH3 comprises the amino acid sequence of SEQ ID NO: 32, the CDRL1 comprises the amino acid sequence of SEQ ID NO: 33, the CDRL2 comprises the amino acid sequence of SEQ ID NO: 34, and the CDRL3 comprises the amino acid sequence of SEQ ID NO: 35.

In one embodiment, provided is an antibody or fragment thereof having binding specificity to a wild-type human signal regulatory protein alpha (SIRPα) protein, wherein the antibody or fragment thereof comprises a heavy chain variable region comprising heavy chain complementarity determining regions CDRH1, CDRH2, and CDRH3 and a light chain variable region light chain comprising complementarity determining regions CDRL1, CDRL2, and CDRL3, and wherein the CDRH1 comprises the amino acid sequence of SEQ ID NO: 30, the CDRH2 comprises the amino acid sequence of SEQ ID NO: 38, the CDRH3 comprises the amino acid sequence of SEQ ID NO: 32, the CDRL1 comprises the amino acid sequence of SEQ ID NO: 33, the CDRL2 comprises the amino acid sequence of SEQ ID NO: 34, and the CDRL3 comprises the amino acid sequence of SEQ ID NO: 35.

In some embodiments, the heavy chain variable region comprises an amino acid sequence selected from the group consisting of SEQ ID NO:3 and 39-44, or a peptide having at least 90% sequence identity to an amino acid sequence selected from the group consisting of SEQ ID NO:3 and 39-44.

In some embodiments, the light chain variable region comprises an amino acid sequence selected from the group consisting of SEQ ID NO:4 and 45-46, or a peptide having at least 90% sequence identity to an amino acid sequence selected from the group consisting of SEQ ID NO:4 and 45-46.

In some embodiments, the heavy chain variable region comprises the amino acid sequence of SEQ ID NO:43 and the light chain variable region comprises the amino acid sequence of SEQ ID NO:45.

In one embodiment, the CDRs are those of 102A10F2, as exemplified in Tables 4B and 4D. In one embodiment, the CDRH1 comprises the amino acid sequence of SEQ ID NO: 47 or a variant thereof having one, two, or three deletions, additions, substitutions or the combinations thereof, the CDRH2 comprises the amino acid sequence of SEQ ID NO: 48, 53 or 54 or a variant thereof having one, two, or three deletions, additions, substitutions or the combinations thereof, the CDRH3 comprises the amino acid sequence of SEQ ID NO: 49 or a variant thereof having one, two, or three deletions, additions, substitutions or the combinations thereof, the CDRL1 comprises the amino acid sequence of SEQ ID NO: 50 or a variant thereof having one, two, or three deletions, additions, substitutions or the combinations thereof, the CDRL2 comprises the amino acid sequence of SEQ ID NO: 51 or a variant thereof having one, two, or three deletions, additions, substitutions or the combinations thereof, and the CDRL3 comprises the amino acid sequence of SEQ ID NO: 52 or a variant thereof having one, two, or three deletions, additions, substitutions or the combinations thereof.

In one embodiment, provided is an antibody or fragment thereof having binding specificity to a wild-type human signal regulatory protein alpha (SIRPα) protein, wherein the antibody or fragment thereof comprises a heavy chain variable region comprising heavy chain complementarity determining regions CDRH1, CDRH2, and CDRH3 and a light chain variable region light chain comprising complementarity determining regions CDRL1, CDRL2, and CDRL3, and wherein the CDRH1 comprises the amino acid sequence of SEQ ID NO: 47, the CDRH2 comprises the amino acid sequence of SEQ ID NO: 48, the CDRH3 comprises the amino acid sequence of SEQ ID NO: 49, the CDRL1 comprises the amino acid sequence of SEQ ID NO: 50, the CDRL2 comprises the amino acid sequence of SEQ ID NO: 51, and the CDRL3 comprises the amino acid sequence of SEQ ID NO: 52.

In one embodiment, provided is an antibody or fragment thereof having binding specificity to a wild-type human signal regulatory protein alpha (SIRPα) protein, wherein the antibody or fragment thereof comprises a heavy chain variable region comprising heavy chain complementarity determining regions CDRH1, CDRH2, and CDRH3 and a light chain variable region light chain comprising complementarity determining regions CDRL1, CDRL2, and CDRL3, and wherein the CDRH1 comprises the amino acid sequence of SEQ ID NO: 47, the CDRH2 comprises the amino acid sequence of SEQ ID NO: 53, the CDRH3 comprises the amino acid sequence of SEQ ID NO: 49, the CDRL1 comprises the amino acid sequence of SEQ ID NO: 50, the CDRL2 comprises the amino acid sequence of SEQ ID NO: 51, and the CDRL3 comprises the amino acid sequence of SEQ ID NO: 52.

In one embodiment, provided is an antibody or fragment thereof having binding specificity to a wild-type human signal regulatory protein alpha (SIRPα) protein, wherein the antibody or fragment thereof comprises a heavy chain variable region comprising heavy chain complementarity determining regions CDRH1, CDRH2, and CDRH3 and a light chain variable region light chain comprising complementarity determining regions CDRL1, CDRL2, and CDRL3, and wherein the CDRH1 comprises the amino acid sequence of SEQ ID NO: 47, the CDRH2 comprises the amino acid sequence of SEQ ID NO: 54, the CDRH3 comprises the amino acid sequence of SEQ ID NO: 49, the CDRL1 comprises the amino acid sequence of SEQ ID NO: 50, the CDRL2 comprises the amino acid sequence of SEQ ID NO: 51, and the CDRL3 comprises the amino acid sequence of SEQ ID NO: 52.

In some embodiments, the heavy chain variable region comprises an amino acid sequence selected from the group consisting of SEQ ID NO:5 and 55-60, or a peptide having at least 90% sequence identity to an amino acid sequence selected from the group consisting of SEQ ID NO:5 and 55-60.

In some embodiments, the light chain variable region comprises an amino acid sequence selected from the group consisting of SEQ ID NO:6 and 61-62, or a peptide having at least 90% sequence identity to an amino acid sequence selected from the group consisting of SEQ ID NO: 6 and 61-62.

In one embodiment, the CDRs are those of 62D2H6, as exemplified in Tables 5B and 5D. In one embodiment, the CDRH1 comprises the amino acid sequence of SEQ ID NO: 63 or a variant thereof having one, two, or three deletions, additions, substitutions or the combinations thereof, the CDRH2 comprises the amino acid sequence of SEQ ID NO: 64 or a variant thereof having one, two, or three deletions, additions, substitutions or the combinations thereof, the CDRH3 comprises the amino acid sequence of SEQ ID NO: 65 or a variant thereof having one, two, or three deletions, additions, substitutions or the combinations thereof, the CDRL1 comprises the amino acid sequence of SEQ ID NO: 66 or a variant thereof having one, two, or three deletions, additions, substitutions or the combinations thereof, the CDRL2 comprises the amino acid sequence of SEQ ID NO: 67 or a variant thereof having one, two, or three deletions, additions, substitutions or the combinations thereof, and the CDRL3 comprises the amino acid sequence of SEQ ID NO: 68 or a variant thereof having one, two, or three deletions, additions, substitutions or the combinations thereof.

In some embodiments, the heavy chain variable region comprises an amino acid sequence selected from the group consisting of SEQ ID NO:7 and 69-72, or a peptide having at least 90% sequence identity to an amino acid sequence selected from the group consisting of SEQ ID NO:7 and 69-72.

In some embodiments, the light chain variable region comprises an amino acid sequence selected from the group consisting of SEQ ID NO:8 and 73-76, or a peptide having at least 90% sequence identity to an amino acid sequence selected from the group consisting of SEQ ID NO: 8 and 73-76.

In one embodiment, the CDRs are those of 211F8E11, as exemplified in Tables 6B and 6D. In one embodiment, the CDRH1 comprises the amino acid sequence of SEQ ID NO: 77 or a variant thereof having one, two, or three deletions, additions, substitutions or the combinations thereof, the CDRH2 comprises the amino acid sequence of SEQ ID NO: 78 or a variant thereof having one, two, or three deletions, additions, substitutions or the combinations thereof, the CDRH3 comprises the amino acid sequence of SEQ ID NO: 79 or a variant thereof having one, two, or three deletions, additions, substitutions or the combinations thereof, the CDRL1 comprises the amino acid sequence of SEQ ID NO: 80 or a variant thereof having one, two, or three deletions, additions, substitutions or the combinations thereof, the CDRL2 comprises the amino acid sequence of SEQ ID NO: 81 or a variant thereof having one, two, or three deletions, additions, substitutions or the combinations thereof, and the CDRL3 comprises the amino acid sequence of SEQ ID NO: 82 or a variant thereof having one, two, or three deletions, additions, substitutions or the combinations thereof.

In some embodiments, the heavy chain variable region comprises an amino acid sequence selected from the group consisting of SEQ ID NO:9 and 83-86, or a peptide having at least 90% sequence identity to an amino acid sequence selected from the group consisting of SEQ ID NO:9 and 83-86.

In some embodiments, the light chain variable region comprises an amino acid sequence selected from the group consisting of SEQ ID NO:10 and 87-90, or a peptide having at least 90% sequence identity to an amino acid sequence selected from the group consisting of SEQ ID NO: 10 and 87-90.

In one embodiment, the CDRs are those of 217D11E5, as exemplified in Tables 7B and 7D. In one embodiment, the CDRH1 comprises the amino acid sequence of SEQ ID NO: 91 or a variant thereof having one, two, or three deletions, additions, substitutions or the combinations thereof, the CDRH2 comprises the amino acid sequence of SEQ ID NO: 92 or a variant thereof having one, two, or three deletions, additions, substitutions or the combinations thereof, the CDRH3 comprises the amino acid sequence of SEQ ID NO: 93 or a variant thereof having one, two, or three deletions, additions, substitutions or the combinations thereof, the CDRL1 comprises the amino acid sequence of SEQ ID NO: 94 or a variant thereof having one, two, or three deletions, additions, substitutions or the combinations thereof, the CDRL2 comprises the amino acid sequence of SEQ ID NO: 95 or a variant thereof having one, two, or three deletions, additions, substitutions or the combinations thereof, and the CDRL3 comprises the amino acid sequence of SEQ ID NO: 96 or a variant thereof having one, two, or three deletions, additions, substitutions or the combinations thereof.

In some embodiments, the heavy chain variable region comprises an amino acid sequence selected from the group consisting of SEQ ID NO:11 and 97-100, or a peptide having at least 90% sequence identity to an amino acid sequence selected from the group consisting of SEQ ID NO:11 and 97-100.

In some embodiments, the light chain variable region comprises an amino acid sequence selected from the group consisting of SEQ ID NO:12 and 101-102, or a peptide having at least 90% sequence identity to an amino acid sequence selected from the group consisting of SEQ ID NO: 12 and 101-102.

In one embodiment, the CDRs are those of 234B7D5, as exemplified in Tables 8B and 8D. In one embodiment, the CDRH1 comprises the amino acid sequence of SEQ ID NO: 103 or a variant thereof having one, two, or three deletions, additions, substitutions or the combinations thereof, the CDRH2 comprises the amino acid sequence of SEQ ID NO: 104 or a variant thereof having one, two, or three deletions, additions, substitutions or the combinations thereof, the CDRH3 comprises the amino acid sequence of SEQ ID NO: 105 or a variant thereof having one, two, or three deletions, additions, substitutions or the combinations thereof, the CDRL1 comprises the amino acid sequence of SEQ ID NO: 106 or a variant thereof having one, two, or three deletions, additions, substitutions or the combinations thereof, the CDRL2 comprises the amino acid sequence of SEQ ID NO: 107 or a variant thereof having one, two, or three deletions, additions, substitutions or the combinations thereof, and the CDRL3 comprises the amino acid sequence of SEQ ID NO: 108 or a variant thereof having one, two, or three deletions, additions, substitutions or the combinations thereof.

In some embodiments, the heavy chain variable region comprises an amino acid sequence selected from the group consisting of SEQ ID NO:13 and 109-112, or a peptide having at least 90% sequence identity to an amino acid sequence selected from the group consisting of SEQ ID NO:13 and 109-112.

In some embodiments, the light chain variable region comprises an amino acid sequence selected from the group consisting of SEQ ID NO:14 and 113-118, or a peptide having at least 90% sequence identity to an amino acid sequence selected from the group consisting of SEQ ID NO: 14 and 113-118.

In some embodiments, the anti-SIRPα antibody specifically binds to SIRPα competitively with any one of the anti-SIRPα antibodies described herein. In some embodiments, competitive binding may be determined using an ELISA assay.

The antibodies that contained these CDR regions, whether mouse, humanized or chimeric, had potent SIRPα binding and inhibitory activities. As shown in Example 5, certain residues within the CDR can be modified to retain or improve the property or reduce their potential to have post-translational modifications (PTMs). Such modified CDR can be referred to as affinity matured or de-risked CDRs.

Non-limiting examples of de-risked CDRs are provided in Tables 2B, 3B and 4B. Modified CDRs can include those having one, two or three amino acid addition, deletion and/or substitutions. In some embodiments, the substitutions can be conservative substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

Non-limiting examples of conservative amino acid substitutions are provided in the table below, where a similarity score of 0 or higher indicates conservative substitution between the two amino acids.

TABLE A
Amino Acid Similarity Matrix

| | C | G | P | S | A | T | D | E | N | Q | H | K | R | V | M | I | L | F | Y | W |
|---|---|---|---|---|---|---|---|---|---|---|---|---|---|---|---|---|---|---|---|---|
| W | −8 | −7 | −6 | −2 | −6 | −5 | −7 | −7 | −4 | −5 | −3 | −3 | 2 | −6 | −4 | −5 | −2 | 0 | 0 | 17 |
| Y | 0 | −5 | −5 | −3 | −3 | −3 | −4 | −4 | −2 | −4 | 0 | −4 | −5 | −2 | −2 | −1 | −1 | 7 | 10 | |
| F | −4 | −5 | −5 | −3 | −4 | −3 | −6 | −5 | −4 | −5 | −2 | −5 | −4 | −1 | 0 | 1 | 2 | 9 | | |
| L | −6 | −4 | −3 | −3 | −2 | −2 | −4 | −3 | −3 | −2 | −2 | −3 | −3 | 2 | 4 | 2 | 6 | | | |
| I | −2 | −3 | −2 | −1 | −1 | 0 | −2 | −2 | −2 | −2 | −2 | −2 | −2 | 4 | 2 | 5 | | | | |
| M | −5 | −3 | −2 | −2 | −1 | −1 | −3 | −2 | 0 | −1 | −2 | 0 | 0 | 2 | 6 | | | | | |
| V | −2 | −1 | −1 | −1 | 0 | 0 | −2 | −2 | −2 | −2 | −2 | −2 | −2 | 4 | | | | | | |
| R | −4 | −3 | 0 | 0 | −2 | −1 | −1 | −1 | 0 | 1 | 2 | 3 | 6 | | | | | | | |
| K | −5 | −2 | −1 | 0 | −1 | 0 | 0 | 0 | 1 | 1 | 0 | 5 | | | | | | | | |
| H | −3 | −2 | 0 | −1 | −1 | −1 | 1 | 1 | 2 | 3 | 6 | | | | | | | | | |
| Q | −5 | −1 | 0 | −1 | 0 | −1 | 2 | 2 | 1 | 4 | | | | | | | | | | |
| N | −4 | 0 | −1 | 1 | 0 | 0 | 2 | 1 | 2 | | | | | | | | | | | |
| E | −5 | 0 | −1 | 0 | 0 | 0 | 3 | 4 | | | | | | | | | | | | |
| D | −5 | 1 | −1 | 0 | 0 | 0 | 4 | | | | | | | | | | | | | |
| T | −2 | 0 | 0 | 1 | 1 | 3 | | | | | | | | | | | | | | |
| A | −2 | 1 | 1 | 1 | 2 | | | | | | | | | | | | | | | |
| S | 0 | 1 | 1 | 1 | | | | | | | | | | | | | | | | |
| P | −3 | −1 | 6 | | | | | | | | | | | | | | | | | |
| G | −3 | 5 | | | | | | | | | | | | | | | | | | |
| C | 12 | | | | | | | | | | | | | | | | | | | |

TABLE B
Conservative Amino Acid Substitutions

| For Amino Acid | Substitution With |
|---|---|
| Alanine | D-Ala, Gly, Aib, ß-Ala, L-Cys, D-Cys |
| Arginine | D-Arg, Lys, D-Lys, Qrn D-Qrn |
| Asparagine | D-Asn, Asp, D-Asp, Glu, D-Glu Gln, D-Gln |
| Aspartic Acid | D-Asp, D-Asn, Asn, Glu, D-Glu, Gln, D-Gln |
| Cysteine | D-Cys, S-Me-Cys, Met, D-Met, Thr, D-Thr, L-Ser, D-Ser |
| Glutamine | D-GIn, Asn, D-Asn, Glu, D-Glu, Asp, D-Asp |
| Glutamic Acid | D-Glu, D-Asp, Asp, Asn, D-Asn, Gln, D-Gln |
| Glycine | Ala, D-Ala, Pro, D-Pro, Aib, ß-Ala |
| Isoleucine | D-Ile, Val, D-Val, Leu, D-Leu, Met, D-Met |
| Leucine | Val, D-Val, Met, D-Met, D-Ile, D-Leu, Ile |
| Lysine | D-Lys, Arg, D-Arg, Qrn, D-Qrn |
| Methionine | D-Met, S-Me-Cys, Ile, D-Ile, Leu, D-Leu, Val, D-Val |
| Phenylalanine | D-Phe, Tyr, D-Tyr, His, D-His, Trp, D-Trp |
| Proline | D-Pro |
| Serine | D-Ser, Thr, D-Thr, allo-Thr, L-Cys, D-Cys |
| Threonine | D-Thr, Ser, D-Ser, allo-Thr, Met, D-Met, Val, D-Val |
| Tyrosine | D-Tyr, Phe, D-Phe, His, D-His, Trp, D-Trp |
| Valine | D-Val, Leu, D-Leu, Ile, D-Ile, Met, D-Met |

### Anti-PD-L1 Unit

The anti-PD-L1 units described herein can include a single-domain antibody (sdAb). Exemplary sdAbs include, but are not limited to, heavy chain variable domains from heavy-chain only antibodies (e.g., V_{H}H (Variable domain of the heavy chain of the Heavy chain antibody) in Camelidae or V_{NAR} (Variable domain of the shark New Antigen Receptor) in cartilaginous fish), binding molecules naturally devoid of light chains, single domains (such as V_{H} or V_{L}) derived from conventional 4-chain antibodies, humanized heavy-chain only antibodies, human single-domain antibodies produced by transgenic mice or rats expressing human heavy chain segments, and engineered domains and single domain scaffolds other than those derived from antibodies. The sdAbs may be derived from any species including, but not limited to mouse, rat, human, camel, llama, lamprey, fish, shark, goat, rabbit, and bovine. Single-domain antibodies contemplated herein also include naturally occurring single-domain antibody molecules from species other than Camelidae and sharks.

In some embodiments, the sdAb is derived from a naturally occurring single-domain antigen binding molecule known as heavy chain antibody devoid of light chains (also referred herein as “heavy chain-only antibodies”, or “HCAb”). Such single domain molecules are disclosed in WO 94/04678 and Hamers-Casterman, C. et al. (1993) Nature 363:446-448, for example. For clarity reasons, the variable domain derived from a heavy chain molecule naturally devoid of light chain is known herein as a V_{H}H to distinguish it from the conventional VH of four chain immunoglobulins. Such a V_{H}H molecule can be derived from antibodies raised in Camelidae species, for example, camel, llama, vicuna, dromedary, alpaca and guanaco. Other species besides Camelidae may produce heavy chain molecules naturally devoid of light chain, and such V_{H}Hs are within the scope of the present application.

In some embodiments, the sdAb is derived from a variable region of the immunoglobulin found in cartilaginous fish. For example, the sdAb can be derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

In some embodiments, the anti-PD-L1 sdAb includes a CDR1 comprising the amino acid sequence of any one of SEQ ID NOs: 169-218, or a variant thereof comprising up to about 3 (such as about any of 1, 2, or 3) amino acid substitutions; a CDR2 comprising the amino acid sequence of any one of SEQ ID NOs: 169-318, or a variant thereof comprising up to about 3 (such as about any of 1, 2, or 3) amino acid substitutions; and a CDR3 comprising the amino acid sequence of any one of SEQ ID NOs: 369-418, or a variant thereof comprising up to about 3 (such as about any of 1, 2, or 3) amino acid substitutions. In some embodiments, the K_{d} of the binding between the anti-PD-L1 sdAb and PD-L1 is about 10⁻⁵ M to about 10⁻¹² M (such as about 10⁻⁷ M to about 10 12 M, or about 10⁻⁸ M to about 10⁻¹² M). In some embodiments, the anti-PD-L1 sdAb is camelid, chimeric, human, partially humanized, or fully humanized.

In some embodiments, the anti-PD-L1 sdAb includes a CDR3 comprising the amino acid sequence of any one of SEQ ID NOs: 369-418, and the amino acid substitutions are in CDR1 and/or CDR2.

Thus, in some embodiments, the anti-PD-L1 sdAb includes a CDR1 comprising the amino acid sequence of any one of SEQ ID NOs: 169-218, or a variant thereof comprising up to about 3 (such as about any of 1, 2, or 3) amino acid substitutions; a CDR2 comprising the amino acid sequence of any one of SEQ ID NOs: 169-318, or a variant thereof comprising up to about 3 (such as about any of 1, 2, or 3) amino acid substitutions; and a CDR3 comprising the amino acid sequence of any one of SEQ ID NOs: 369-418. In some embodiments, the K_{d} of the binding between the anti-PD-L1 sdAb and PD-L1 is about 10⁻⁵ M to about 10⁻¹² M (such as about 10⁻⁷ M to about 10⁻¹² M, or about 10⁻⁸ M to about 10⁻¹² M). In some embodiments, the anti-PD-L1 sdAb is camelid, chimeric, human, partially humanized, or fully humanized.

In some embodiments, the anti-PD-L1 sdAb includes a CDR1 comprising the amino acid sequence of any one of SEQ ID NOs: 169-218; a CDR2 comprising the amino acid sequence of any one of SEQ ID NOs: 169-318; and a CDR3 comprising the amino acid sequence of any one of SEQ ID NOs: 369-418. In some embodiments, the K_{d} of the binding between the anti-PD-L1 sdAb and PD-L1 is about 10⁻⁵ M to about 10⁻¹² M (such as about 10⁻⁷ M to about 10⁻¹² M, or about 10⁻⁸ M to about 10⁻¹² M). In some embodiments, the anti-PD-L1 sdAb is camelid, chimeric, human, partially humanized, or fully humanized.

The sequences of the CDRs noted herein are provided in Table 24.

The CDRs can be combined in various pair-wise combinations to generate a number of anti-PD-L1 sdAb.

The anti-PD-L1 sdAb may comprise one or more “hallmark residues” in one or more of the FR sequences. In some embodiments, the anti-PD-L1 sdAb may comprise a V_{H}H domain comprising the amino acid sequence of any one of the following: a-1) the amino acid residue at position 37 is selected from the group consisting of F, Y, L, I, and V (such as Y or such as F); a-2) the amino acid residue at position 44 is selected from the group consisting of A, G, E, D, G, Q, R, S, and L (such as G, E, or Q); a-3) the amino acid residue at position 45 is selected from the group consisting of L, R and C (such as L or R); a-4) the amino acid residue at position 103 is selected from the group consisting of G, W, R and S (such as W or R, or such as W); and a-5) the amino acid residue at position 108 is Q; or b-1) the amino acid residue at position 37 is selected from the group consisting of F, Y, L, I, and V (such as Y or such as F); b-2) the amino acid residue at position 44 is selected from the group consisting of E and Q; b-3) the amino acid residue at position 45 is R; b-4) the amino acid residue at position 103 is selected from the group consisting of G, W, R and S (such as W); and b-5) the amino acid residue at position 108 is selected from the group consisting of Q and L (such as Q); wherein the amino acid position is according to Kabat numbering. It should be noted that these “hallmark residues” at amino acid positions 37, 44, 45, 103 and 108 according to Kabat numbering apply to anti-PD-L1 sdAb of natural V_{H}H sequences, and can be substituted during humanization. For example, Q at amino acid position 108 according to Kabat numbering can be optionally humanized to L. Other humanized substitutions will be clear to those skilled in the art. For example, potentially useful humanizing substitutions can be determined by comparing the FR sequences of a naturally occurring V_{H}H with the corresponding FR sequences of one or more closely related human V_{H}, then introducing one or more of such potentially useful humanizing substitutions into said V_{H}H using methods known in the art (also as described herein). The resulting humanized V_{H}H sequences can be tested for their PD-L1 binding affinity, for stability, for ease and level of expression, and/or for other desired properties. Possible residue substitutions may also come from an antibody V_{H} domain wherein the VH/VL interface comprises one or more highly charged amino acid residues. The anti-PD-L1 sdAb described herein can be partially or fully humanized. Preferably, the resulting humanized anti-PD-L1 sdAb binds to PD-L1 with K_{d}, Kₒₙ, K_{off} described herein.

In some embodiments, the anti-PD-L1 sdAb includes a V_{H}H domain comprising the amino acid sequence of any one of SEQ ID NOs: 469-518, or a variant thereof having at least about 80% (such as at least about any of 80%, 85%, 90%, 91%, 92%, 93%, 94%, 95%, 96%, 97%, 98%, or 99%) sequence identify to any one of SEQ ID NOs:469-518. In some embodiments, the anti-PD-L1 sdAb includes a V_{H}H domain comprising the amino acid sequence of any one of SEQ ID NOs: 469-518, or a variant thereof comprising up to about 3 (such as about any of 1, 2, or 3) amino acid substitutions in the V_{H}H domain. In some embodiments, the anti-PD-L1 sdAb includes the V_{H}H domain comprising the amino acid sequence of any one of SEQ ID NOs: 469-518 or a variant thereof comprises amino acid substitutions in CDRs, such as the CDR1, and/or the CDR2, and/or the CDR3 of any one of SEQ ID NOs: 469-518. In some embodiments, the anti-PD-L1 sdAb includes the V_{H}H domain comprising the amino acid sequence of any one of SEQ ID NOs: 469-518 or a variant thereof comprises CDR1, CDR2, and CDR3 of any one of SEQ ID NOs: 469-518, and the amino acid substitutions are in FRs, such as the FR1, and/or the FR2, and/or the FR3, and/or the FR4 of any one of SEQ ID NOs: 469-518.

In some embodiments, the anti-PD-L1 sdAb specifically binds to PD-L1 competitively with any one of the anti-PD-L1 sdAb described herein. In some embodiments, competitive binding may be determined using an ELISA assay. For example, in some embodiments, there is provided an anti-PD-L1 sdAb that specifically binds to PD-L1 competitively with an anti-PD-L1 sdAb comprising the amino acid sequence of any one of SEQ ID NOs: 469-518. For another example, in some embodiments, there is provided an anti-PD-L1 sdAb that specifically binds to PD-L1 competitively with an anti-PD-L1 sdAb comprising a CDR1 comprising the amino acid sequence of any one of SEQ ID NOs: 169-218; a CDR2 comprising the amino acid sequence of any one of SEQ ID NOs: 169-318; and a CDR3 comprising the amino acid sequence of any one of SEQ ID NOs: 369-418. In some embodiments, the K_{d} of the binding between the competing anti-PD-L1 sdAb and PD-L1 is about 10⁻⁵ M to about 10⁻¹² M (such as about 10⁻⁷ M to about 10⁻¹² M, or about 10⁻⁸ M to about 10⁻¹² M). In some embodiments, the competing anti-PD-L1 sdAb is camelid, chimeric, human, partially humanized, or fully humanized.

In a preferred embodiment, the CDR1 comprises the amino acid sequence of SEQ ID NO:213, the CDR2 comprises the amino acid sequence of SEQ ID NO:313, and the CDR3 comprises the amino acid sequence of SEQ ID NO:413. In some embodiments, the anti-PD-L1 sdAb comprises the amino acid sequence of SEQ ID NO:513.

Example sequences of the bispecific antibodies are also provided. For instance, the bispecific antibody may include (a) a heavy chain comprising the amino acid sequence of SEQ ID NO:559 and a light chain comprising the amino acid sequence of SEQ ID NO:560; (b) a heavy chain comprising the amino acid sequence of SEQ ID NO:561 and a light chain comprising the amino acid sequence of SEQ ID NO:562; (c) a heavy chain comprising the amino acid sequence of SEQ ID NO:563 and a light chain comprising the amino acid sequence of SEQ ID NO:560; (d) a heavy chain comprising the amino acid sequence of SEQ ID NO:564 and a light chain comprising the amino acid sequence of SEQ ID NO:562; (e) a heavy chain comprising the amino acid sequence of SEQ ID NO:565 and a light chain comprising the amino acid sequence of SEQ ID NO:566; (f) a heavy chain comprising the amino acid sequence of SEQ ID NO:567 and a light chain comprising the amino acid sequence of SEQ ID NO:568; (g) a heavy chain comprising the amino acid sequence of SEQ ID NO:565 and a light chain comprising the amino acid sequence of SEQ ID NO:569; or (h) a heavy chain comprising the amino acid sequence of SEQ ID NO:567 and a light chain comprising the amino acid sequence of SEQ ID NO:570.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence.

In certain embodiments, the antibody comprises an amino acid sequence or one or more not normally associated with an antibody. Exemplary modifications are described in more detail below. For example, an antibody of the disclosure may comprise a flexible linker sequence, or may be modified to add a functional moiety (e.g., PEG, a drug, a toxin, or a label).

Antibodies, variants, or derivatives thereof of the disclosure include derivatives that are modified, i.e., by the covalent attachment of any type of molecule to the antibody such that covalent attachment does not prevent the antibody from binding to the epitope. For example, but not by way of limitation, the antibodies can be modified, e.g., by glycosylation, acetylation, pegylation, phosphorylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

In some embodiments, the antibodies may be conjugated to therapeutic agents, prodrugs, peptides, proteins, enzymes, viruses, lipids, biological response modifiers, pharmaceutical agents, or PEG.

The antibodies may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

The antibodies can be detectably labeled by coupling it to a chemiluminescent compound. The presence of the chemiluminescent-tagged antigen-binding polypeptide is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

The antibodies can also be detectably labeled using fluorescence emitting metals such as ¹⁵²Eu, or others of the lanthanide series. These metals can be attached to the antibody using such metal chelating groups as diethylenetriaminepentacetic acid (DTPA) or ethylenediaminetetraacetic acid (EDTA). Techniques for conjugating various moieties to an antibody are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al., (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. (52:119-58 (1982)).

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Treatment Methods

As described herein, the antibodies, variants or derivatives of the present disclosure may be used in certain treatment and diagnostic methods.

The present disclosure is further directed to antibody-based therapies which involve administering the antibodies of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

The antibodies of the disclosure can also be used to treat or inhibit cancer. As provided above, SIRPα can be overexpressed in tumor cells, in particular gastric, pancreatic, esophageal, ovarian, and lung tumors. Inhibition of SIRPα has been shown to be useful for treating the tumors. Some tumors may also overexpress PD-L1 or PD-1, or can be induced to overexpress PD-L1 or PD-1. All of the tumors, it is contemplated, can be effectively treated with the antibodies of the present disclosure.

Accordingly, in some embodiments, provided are methods for treating a cancer in a patient in need thereof. The method, in one embodiment, entails administering to the patient an effective amount of an antibody of the present disclosure. In some embodiments, at least one of the cancer cells (e.g., stromal cells) in the patient over-express SIRPα, CD47, PD-1 or PD-L1.

Cellular therapies, such as chimeric antigen receptor (CAR) T-cell therapies, are also provided in the present disclosure. A suitable cell can be used, that is put in contact with an anti-SIRPα antibody of the present disclosure (or alternatively engineered to express an anti-SIRPα antibody of the present disclosure). Upon such contact or engineering, the cell can then be introduced to a cancer patient in need of a treatment. The cancer patient may have a cancer of any of the types as disclosed herein. The cell (e.g., T cell) can be, for instance, a tumor-infiltrating T lymphocyte, a CD4+ T cell, a CD8+ T cell, or the combination thereof, without limitation.

In some embodiments, the cell was isolated from the cancer patient him- or her-self. In some embodiments, the cell was provided by a donor or from a cell bank. When the cell is isolated from the cancer patient, undesired immune reactions can be minimized.

Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer. In some embodiments, the cancer is one or more of gastric, pancreatic, esophageal, ovarian, and lung cancers.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibodies, variants or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antigen-binding polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

The amount of the antibodies of the disclosure which will be effective in the treatment, inhibition and prevention of an inflammatory, immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antigen-binding polypeptides of the present disclosure is typically 0.1 mg/kg to 100 mg/kg of the patient's body weight, between 0.1 mg/kg and 20 mg/kg of the patient's body weight, or 1 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions of the disclosure are administered in combination with other therapeutic or prophylactic regimens, such as, for example, radiation therapy.

### Compositions

The present disclosure also provides pharmaceutical compositions. Such compositions comprise an effective amount of an antibody, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1

### Generation of Murine Monoclonal Antibodies Against Human SIRPα

The human SIRPα protein was used to immunize different strains of mice and hybridomas were generated accordingly. Eight fusions were made to generate sufficient number of hybridoma clones. SIRPα v1/v2 positive binders were selected and subcloned. Subsequently, in vitro binding and functional screening were carried out with about 30 purified antibodies and lead antibodies with highest binding affinity and strongest functional potency were identified. The lead antibodies were humanized.

The VH/VL sequences of the lead murine antibodies are provided in the table below.

TABLE 1
VH/VL sequence of the lead murine antibodies

| Name | Sequence (CDRs are underlined) | SEQ ID NO: |
|---|---|---|
| 248G3F6 | EVQLQQSGAELVKPGASVKLSCTASGENFEDTYMHWVKQRPDQGLEWIGR | 1 |
| VH | IDPADGDTKYNPKFQDKATITVDTSSNTAYLQLSSLISEDTAVYYCVRGN | |
| | YVNWGQGTTLTVSS | |
| 248G3F6 | QIVLIQSPAIMSASPGERVILTCRASSSVSSSYLYWYQQKPGSSPKLWIY | 2 |
| VL | STSNLASGVPARFSGSGSGTSYSLTISSMEAEDAASYFCHQWYSYPRTFG | |
| | GGTKLEIK | |
| 300A6A6 | QVQLQQSGTELVKPGSSVKISCKASGYTFTSNYIHWIRQQPGNGLEWIGW | 3 |
| VH | IYPGDGDTNYNQKFNGKATLTADKSSSTAYMQLSSLTSEDYAVYFCAINY | |
| | GGIWFAYWGQGTLVTVSS | |
| 300A6A6 | DIQMTQSPSSMSASLGDRVTITCQASQDIGNKLIWFQQKPGKSPRIMIHY | 4 |
| VL | VTNLPGGVPLRFSGSRSGSDYSLTISSLESEDMADYYCLQYKQNPLTFGS | |
| | GTKLEIK | |
| 102A10F2 | QVTLKESGPGILQPSQTLSLTCSESGFSLNTYDIGMGWIRQPSGKGLEWL | 5 |
| VH | AHIWWNDREYYNSALQSRVTISKDTSNTQVFLKIASVDTADTATYYCVRI | |
| | DYFGSGQAWFTYWGQGTLVTVSA | |
| 102A10F2 | EIVLTQSPPTMAASPGEKITITCSSSSTISSTYLHWYQQKPGESPKLLIS | 6 |
| VL | GTSNLASGVPPRFSGSGSGTSYSLTIGTLEAEDVATYYCQQGSRIPFTFG | |
| | SGTKLEIK | |
| 62D2H6 | EVQLQQSGAELVKPGASVKLSCTASGENIKDYYMHWVKQRTEQGLEWIGR | 7 |
| VH | IDPEDGETKYAPKFQGKATITADTSSNTAYLQLSSLISEDTAVYYCSRSW | |
| | AYWGQGTTLTVSS | |
| 62D2H6 | QIVLTQSPAIMSASPGEKVTLTCSASSSVSSSYLYWYQQKPGSSPKLWIY | 8 |
| VL | STSNLASGVPARFSGSGSGTSYSLTISSMEAEDAASYFCHQWSSYPRTFG | |
| | GGTKLEIK | |
| 211F8E11 | EVQLQQSGAELVKPGASVKLSCTASGFNIKDTYMHWVKQRPEQGLEWVGR | 9 |
| VH | IDPANVNTIYDPKFQGKATITADTSSNTAYLQLSSLISEDTAVYYCARVG | |
| | AYDGYDFDYWGQGTTLTVSS | |
| 211F8E11 | DIVLTQSPASLAVSLGQRATISCRASESVDNYGNSEMHWYQQKPGQPPKL | 10 |
| VL | LIYRASNLESGIPARFSGSGSRIDFTLTINPVEADDVATYYCQQNNEDPL | |
| | TFGAGTKLELK | |
| 217D11E5 | EVQLQQSGPELVKPGASVKMSCKASGYTFTSYVMHWVKQKPGQGLEWIGY | 11 |
| VH | INPYNDGTKYNEKFKGKATLISDKSSSTAYMELSSLTSEDSAVYYCARSY | |
| | YDYDGSEDYWGQGTTLTVSS | |
| 217D11E5 | DIVMTQSHKFMSTSVGDRVSITCKASQDVTTAVAWYQQKSGQSPKLLIYS | 12 |
| VL | ASYRYTGDPDRETGSGSGTDFTFTISSVQAEDLAVYYCQQHYSTPWTFGG | |
| | GTKLEIK | |
| 234B7D5 | EVQLQQSGAELVKPGASVKLSCTASGENFEDTYIHWVKQRPDQGLEWIGR | 13 |
| VH | IDPADGDTKHNPKFHDKATVTVDTSSNTAYLELSSLTSEDTAVYYCVRGN | |
| | YVNWGQGTTLTVSS | |
| 234B7D5 | QIVLIQSPAIMSASPGERVILTCRASSSVTSSYLYWYQQKPGSSPKLWIY | 14 |
| VL | SASNLASGVPARFSGSGSGTSYSLTISSVEAEDAASYFCHQWYSYPRTFG | |
| | GGTKLEIK | |

### Example 2

### Cross-Binding of SIRPα v1 and v2

This example measured the dose response of ELISA binding of mouse anti-SIRPα mAb to recombinant human SIRPα variant 1 and variant 2 protein (0.5 μg/ml@100 μl). Recombinant human SIRPα v1 or v2 protein (Biointron) was coated at 0.5 μg/ml in PBS onto microtiter plates for 2 h at RT. After coating of the antigen, the wells were blocked with PBS/0.05% Tween (PBST) with 1% BSA for 1 h at RT.

After washing of the wells with PBST, different concentrations of anti-SIRPα antibodies were added to the well and incubated for 1 at RT. For detection of the binding antibodies, the HRP conjugated secondary antibodies against mouse Fc (Jackson Immuno Research) were added followed by the addition of fluorogenic substrates (Roche). Between all incubation steps, the wells of the plate were washed with PBST three times. Fluorescence was measured in a TECAN Spectrafluor plate reader.

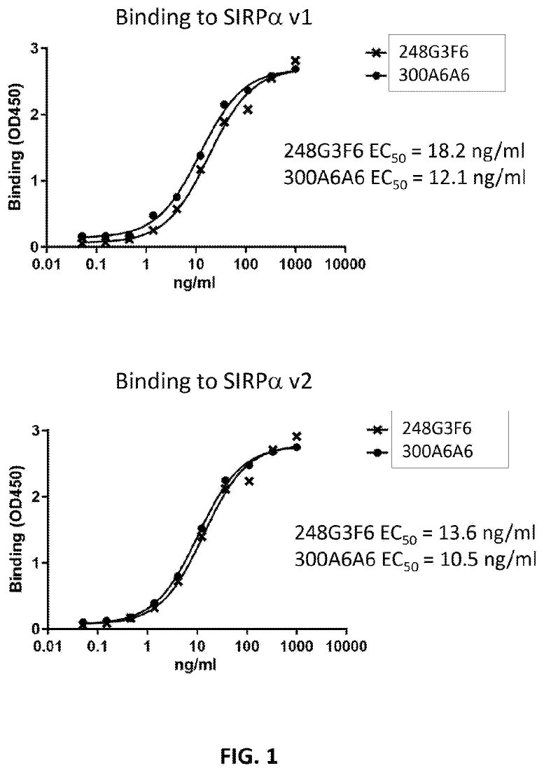

The results are shown in FIG. 1. Both tested antibodies, 248G3F6 and 300A6A6, exhibited nanogram level affinity to both variants 1 and 2.

The binding kinetics assay of antibody to variant 1 was performed using Biacore 8K system through human antibody capture approach. The anti-mouse Fc IgG were immobilized on CM5 sensor chip according to the manufactory's instruction. The test antibody was injected and captured by the immobilized anti-human Fc IgG. Serial concentrations of antigen was individually injected, and the binding profile was recorded for each concentration antigen analyte, respectively.

The assay system was regenerated by injection of 10 mM Glycine-HCl pH 1.5 for 30 seconds. The running buffer was HBS-EP+ (10 mM HEPES, pH 7.4, 150 mM NaCl, 3 mM EDTA and 0.05% P20). The assay temperature was 25° C., and the association and dissociation time were 180 and 600 seconds, respectively. The Biacore data were fitted using Biacore K8 evaluation software 1.0 according to 1:1 binding model to calculate the association (ka) and dissociation (kd) rate constants as well as the equilibrium constant (KD).

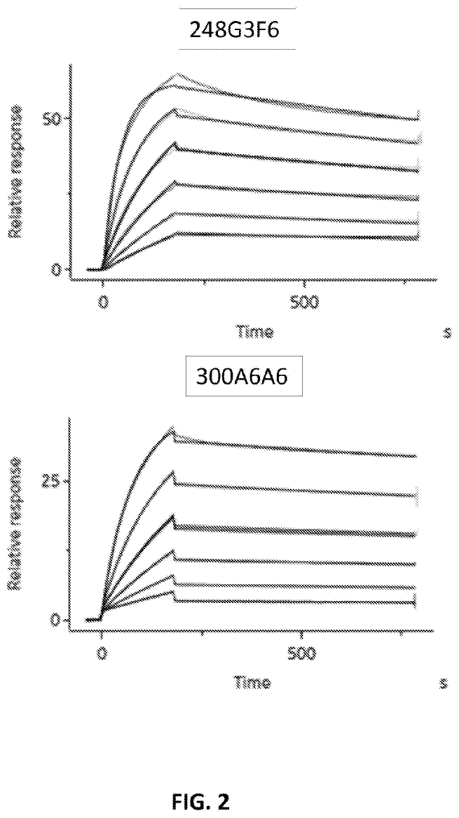

The results are shown in FIG. 2, and summarized in the table below. Both tested antibodies exhibited excellent binding affinity.

| Sample | ka (1/Ms) | kd (1/s) | KD (M) |
|---|---|---|---|
| 248G3F6 | 1.87E+05 | 3.74E−04 | 2.00E−09 |
| 300A6A6 | 1.31E+05 | 1.48E−04 | 1.13E−09 |

### Example 3

### Competition With CD47

This example tested the ability of the anti-SIRPα antibodies to compete with CD47 in binding to SIRPα.

Recombinant CD47-Fc fusion protein (Acrobiosystems) was coated at 1 μg/ml in PBS onto microtiter plates for 16 hours at 4° C. After blocking for 1 h with 1% BSA in PBST at RT, 1 μg/mL of SIRPα-His protein was added either in the absence or presence of different concentrations of anti-SIRPα antibodies at RT for 1 h. Plates were subsequently washed three times and incubated with an HRP-conjugated anti-His secondary antibody for 1 h at RT. After washing, the TMB solution was added to each well for 30 min and the reaction was stopped with 2M H₂SO₄, and OD was measured at 490 nm.

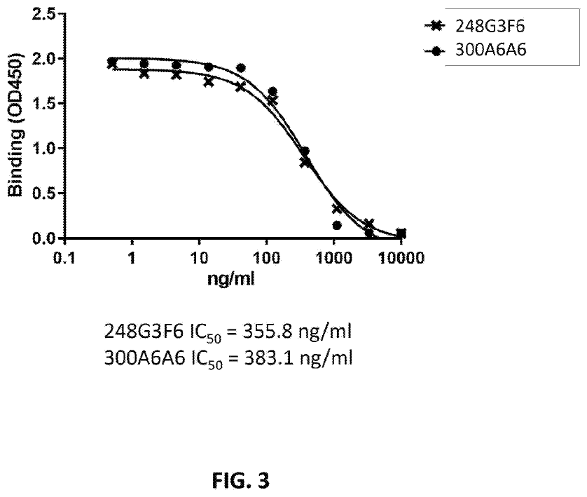

As shown in FIG. 3, both 248G3F6 and 300A6A6 potently and dose-dependently inhibited the binding of CD47 to SIRPα.

### Example 4

### Induction of Macrophage Mediated Phagocytosis

This example tested the ability of the anti-SIRPα antibodies to induce macrophage mediated phagocytosis.

PBMCs were isolated from human blood, and the monocytes were differentiated into macrophages for 6 days. The monocyte derived macrophages (MDMs) were scraped and re-plated in 24-well dishes and allowed to adhere for 24 hours. The human tumor cell line Raji which endogenously expressed CD47 were transfected with human PD-L1 to overexpress human PD-L1 on the surface. This PD-L1 overexpressed Raji cells were chosen as target cells and labeled with 1 μM CFSE for 10 minutes, then added to MDMs at a ratio of 5:1 tumor cells per phagocyte.

Anti-SIRPalpha antibodies and anti-PD-L1 antibody were added in the culture system. After incubation for 3 hours, non-phagocytosed target cells were washed away with PBS and the remaining phagocytes were scraped off, stained with macrophage marker CD14 antibody, and analyzed by flow cytometry. Phagocytosis was measured by gating on CD14⁺ cells and then assessing the percent of CFSE⁺ cells.

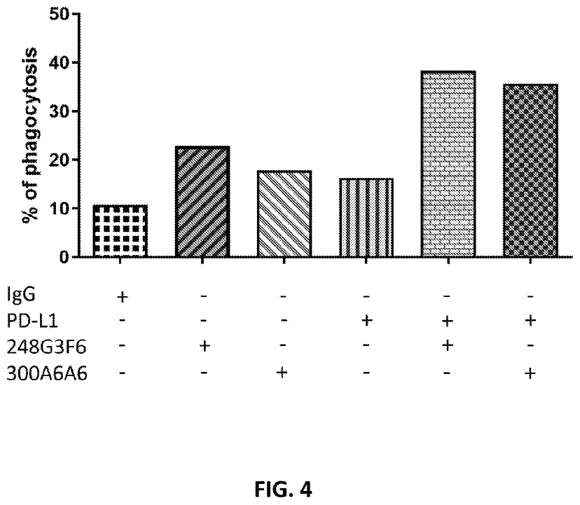

The results of phagocytosis of PD-L1 expressing tumor cells by combo-treatment of anti-SIRPα antibody with anti-PD-L1 antibody are shown in FIG. 4. The combination of anti-PD-L1 antibody with either of the anti-SIRPα antibodies exhibited the highest phagocytosis (the two columns on the right).

### Example 5

### Humanization of the Mouse mAbs

The murine antibody variable region genes were employed to create humanized mAbs. In the first step of this process, the amino acid sequences of the VH and VL of mAb were compared against the available database of human Ig gene sequences to find the overall best-matching human germline Ig gene sequences.

The amino acid sequences of the humanized antibody are provided below.

### Humanized Sequences

### A. 248G3F6

TABLE 2A
Humanization of 248G3F6-VH

| Name | Sequence | SEQ ID NO: |
|---|---|---|
| 248G3F6 VH | EVQLQQSGAELVKPGASVKLSCTASGENFEDTYMHWVKQRPDQGLEWIGR | 1 |
| | IDPADGDTKYNPKFQDKATITVDTSSNTAYLQLSSLISEDTAVYYCVRGN | |
| | YVNWGQGTTLTVSS | |
| V1 (CDR | QVQLVQSGAEVKKPGASVKVSCKASGENFEDTYMHWVRQAPGQGLEWMGR | 23 |
| grafting) | IDPADGDTKYNPKFQDRVTMTRDTSTSTVYMELSSLRSEDTAVYYCARGN | |
| | YVNWGQGTTVTVSS | |
| V2 (with back | QVQLVQSGAEVKKPGASVKVSCKASGENFEDTYMHWVRQAPGQGLEWMGR | 24 |
| mutations) | IDPADGDTKYNPKFQDRVTMTVDTSTNTAYMELSSLRSEDTAVYYCVRGN | |
| | YVNWGQGTTVTVSS | |
| V3 (with back | QVQLVQSGAEVKKPGASVKVSCKASGENFEDTYMHWVRQAPGQGLEWMGR | 25 |
| mutations) | IDPADGDTKYNPKFQDRVTITVDTSTNTAYMELSSLRSEDTAVYYCVRGN | |
| | YVNWGQGTTVTVSS | |
| V4 (with back | QVQLVQSGAEVKKPGASVKVSCKASGENFEDTYMHWVRQAPGQGLEWMGR | 26 |
| mutations) | IDPAEGDTKYNPKFQDRVTITVDTSTNTAYMELSSLRSEDTAVYYCVRGN | |
| | YVNWGQGTTVTVSS | |
| V5 (with back | QVQLVQSGAEVKKPGASVKVSCKASGENFEDTYMHWVRQAPGQGLEWMGR | 27 |
| mutations) | IDPADADTKYNPKFQDRVTITVDTSINTAYMELSSLRSEDTAVYYCVRGN | |
| | YVNWGQGTTVTVSS | |

TABLE 2B
CDR Sequences

| CDR | Sequence | SEQ ID NO: |
|---|---|---|
| CDR-H1 | DTYMH | 15 |
| CDR-H2 | RIDPADGDTKYNPKFQD | 16 |
| CDR-H3 | GNYVN | 17 |
| CDR-H2 (v4) | RIDPAEGDTKYNPKFQD | 21 |
| CDR-H2 (v5) | RIDPADADTKYNPKFQD | 22 |

TABLE 2C
Humanization of 248G3F6-VL

| Name | Sequence | SEQ ID NO: |
|---|---|---|
| 248G3F6 VL | QIVLIQSPAIMSASPGERVTLTCRASSSVSSSYLYWYQQKPGSSPKLWIY | 2 |
| | STSNLASGVPARFSGSGSGTSYSLTISSMEAEDAASYFCHQWYSYPRTFG | |
| | GGTKLEIK | |
| V1 (CDR | EIVLTQSPGTLSLSPGERATLSCRASSSVSSSYLYWYQQKPGQAPRLLIY | 28 |
| grafting) | STSNLASGIPDRESGSGSGTDETLTISRLEPEDFAVYYCHQWYSYPRTFG | |
| | GGTKVEIK | |
| V2 (with back | EIVLTQSPGTLSLSPGERATLSCRASSSVSSSYLYWYQQKPGQAPRLLIY | 29 |
| mutations) | STSNLASGIPDRESGSGSGTDYTLTISRLEPEDAAVYFCHQWYSYPRTFG | |
| | GGTKVEIK | |

TABLE 2D
CDR Sequences

| CDR | Sequence | SEQ ID NO: |
|---|---|---|
| CDR-L1 | RASSSVSSSYLY | 18 |
| CDR-L2 | STSNLAS | 19 |
| CDR-L3 | HQWYSYPRT | 20 |

TABLE 2E
Humanized antibodies

| | VL | VL v1 | VL v2 |
|---|---|---|---|
| VH | HSP210-02-Chi | | |
| VH v1 | | HSP210-02-hz11 | HSP210-02-hz12 |
| VH v2 | | HSP210-02-hz21 | HSP210-02-hz22 |
| VH v3 | | HSP210-02-hz31 | HSP210-02-hz32 |
| VH v4 | | HSP210-02-hz41 | HSP210-02-hz42 |
| VH v5 | | HSP210-02-hz51 | HSP210-02-hz52 |

### B. 300A6A6

TABLE 3A
Humanization of 300A6A6-VH

| Name | Sequence | SEQ ID NO: |
|---|---|---|
| 300A6A6 VH | QVQLQQSGTELVKPGSSVKISCKASGYTFTSNYIHWIRQQPGNGLEWIGW | 3 |
| | IYPGDGDTNYNQKFNGKATLTADKSSSTAYMQLSSLTSEDYAVYFCAINY | |
| | GGIWFAYWGQGTLVTVSS | |
| V1 (CDR | QVQLVQSGAEVKKPGSSVKVSCKASGYTFTSNYIHWVRQAPGQGLEWMGW | 39 |
| grafting) | IYPGDGDTNYNQKFNGRVTITADKSTSTAYMELSSLRSEDTAVYYCARNY | |
| | GGIWFAYWGQGTLVTVSS | |
| V2 (with back | QVQLVQSGAEVKKPGSSVKVSCKASGYTFTSNYIHWVRQAPGQGLEWMGW | 40 |
| mutations) | IYPGDGDTNYNQKENGRVTITADKSTSTAYMELSSLRSEDTAVYYCAINY | |
| | GGIWFAYWGQGTLVTVSS | |
| V3 (with back | QVQLVQSGAEVKKPGSSVKVSCKASGYTFTSNYIHWVRQAPGQGLEWMGW | 41 |
| mutations) | IYPGDGDTNYNQKENGRVTLTADKSTSTAYMELSSLRSEDTAVYYCAINY | |
| | GGIWFAYWGQGTLVTVSS | |
| V4 (with back | QVQLVQSGAEVKKPGSSVKVSCKASGYTFTSNYIHWVRQAPGQGLEWMGW | 42 |
| mutations) | IYPGEGDTNYNQKENGRVTLTADKSTSTAYMELSSLRSEDTAVYYCAINY | |
| | GGIWFAYWGQGTLVTVSS | |
| V5 (with back | QVQLVQSGAEVKKPGSSVKVSCKASGYTFTSNYIHWVRQAPGQGLEWMGW | 43 |
| mutations) | IYPGDADTNYNQKENGRVTLTADKSTSTAYMELSSLRSEDTAVYYCAINY | |
| | GGIWFAYWGQGTLVTVSS | |
| V6 (with back | QVQLVQSGAEVKKPGSSVKVSCKASGYTFTSNYIHWVRQAPGQGLEWMGW | 44 |
| mutations) | IYPGDGDTNYNQKFQGRVTLTADKSTSTAYMELSSLRSEDTAVYYCAINY | |
| | GGIWFAYWGQGTLVTVSS | |

TABLE 3B
CDR Sequences

| CDR | Sequence | SEQ ID NO: |
|---|---|---|
| CDR-H1 | SNYIH | 30 |
| CDR-H2 | WIYPGDGDTNYNQKFNG | 31 |
| CDR-H3 | NYGGIWFAY | 32 |
| CDR-H2 (v4) | WIYPGEGDTNYNQKFNG | 36 |
| CDR-H2 (v5) | WIYPGDADTNYNQKFNG | 37 |
| CDR-H2 (v6) | WIYPGDGDTNYNQKFQG | 38 |

TABLE 3C
Humanization of 300A6A6-VL

| Name | Sequence | SEQ ID NO: |
|---|---|---|
| 300A6A6 VL | DIQMTQSPSSMSASLGDRVTITCQASQDIGNKLIWFQQKPGKSPRLMIHY | 4 |
| | VTNLPGGVPLRESGSRSGSDYSLTISSLESEDMADYYCLQYKQNPLTFGS | |
| | GTKLEIK | |
| V1 (CDR | DIQMTQSPSSLSASVGDRVTITCQASQDIGNKLIWYQQKPGKAPKLLIYY | 45 |
| grafting) | VTNLPGGVPSRFSGSGSGTDFTLTISSLQPEDFATYYCLQYKQNPLTFGQ | |
| | GTKLEIK | |
| V2 (with back | DIQMTQSPSSLSASVGDRVTITCQASQDIGNKLIWFQQKPGKAPKLLIHY | 46 |
| mutations) | VTNLPGGVPSRESGSRSGSDYTLTISSLQPEDFATYYCLQYKQNPLTFGQ | |
| | GTKLEIK | |

TABLE 3D
CDR Sequences

| CDR | Sequence | SEQ ID NO: |
|---|---|---|
| CDR-L1 | QASQDIGNKLI | 33 |
| CDR-L2 | YVTNLPG | 34 |
| CDR-L3 | LQYKQNPLT | 35 |

TABLE 3E
Humanized antibodies

| | VL | VL v1 | VL v2 |
|---|---|---|---|
| VH | HSP210-03-Chi | | |
| VH v1 | | HSP210-03-hz11 | HSP210-03-hz12 |
| VH v2 | | HSP210-03-hz21 | HSP210-03-hz22 |
| VH v3 | | HSP210-03-hz31 | HSP210-03-hz32 |
| VH v4 | | HSP210-03-hz41 | HSP210-03-hz42 |
| VH v5 | | HSP210-03-hz51 | HSP210-03-hz52 |
| VH v6 | | HSP210-03-hz61 | HSP210-03-hz62 |

### C. 102A10F2

TABLE 4A
Humanization of 102A10F2-VH

| Name | Sequence | SEQ ID NO: |
|---|---|---|
| 102A10F2 VH | QVTLKESGPGILQPSQTLSLICSFSGFSLNTYDIGMGWIRQPSGKGLEWLAH | 5 |
| | IWWNDREYYNSALQSRVTISKDTSNTQVFLKIASVDTADTATYYCVRIDYFG | |
| | SGQAWFTYWGQGTLVTVSA | |
| V1 (CDR | QLQLQESGPGLVKPSETLSLTCTVSGFSLNTYDIGMGWIRQPPGKGLEWIGH | 55 |
| grafting) | IWWNDREYYNSALQSRVTISVDISKNQFSLKLSSVTAADTAVYYCARIDYFG | |
| | SGQAWFTYWGQGTLVTVSS | |
| V2 (with back | QVQLQESGPGLVKPSETLSLTCTFSGFSLNTYDIGMGWIRQPPGKGLEWIGH | 56 |
| mutations) | IWWNDREYYNSALQSRVTISKDTSKTQVSLKLSSVTAADTAVYYCVRIDYFG | |
| | SGQAWFTYWGQGTLVTVSS | |
| V3 (with back | QVQLQESGPGLVKPSETLSLTCTFSGESLNTYDIGMGWIRQPPGKGLEWIAH | 57 |
| mutations) | IWWNDREYYNSALQSRVTISKDTSKTQVSLKLSSVTAADTAVYYCVRIDYFG | |
| | SGQAWFTYWGQGTLVTVSS | |
| V4 (with back | QVQLQESGPGLVKPSETLSLTCTFSGESLSTYDIGMGWIRQPPGKGLEWIAH | 58 |
| mutations) | IWWNDREYYNSALQSRVTISKDTSKTQVSLKLSSVTAADTAVYYCVRIDYFG | |
| | SGQAWFTYWGQGTLVTVSS | |
| V5 (with back | QVQLQESGPGLVKPSETLSLTCTFSGFSLNTYDIGMGWIRQPPGKGLEWIAH | 59 |
| mutations) | IWWNDREYYSSALQSRVTISKDTSKTQVSLKLSSVTAADTAVYYCVRIDYFG | |
| | SGQAWFTYWGQGTLVTVSS | |
| V6 (with back | QVQLQESGPGLVKPSETLSLTCTFSGFSLNTYDIGMGWIRQPPGKGLEWIAH | 60 |
| mutations) | IWWNDREYYNPALQSRVTISKDTSKTQVSLKLSSVTAADTAVYYCVRIDYFG | |
| | SGQAWFTYWGQGTLVTVSS | |

TABLE 4B
CDR Sequences

| CDR | Sequence | SEQ ID NO: |
|---|---|---|
| CDR-H1 | TYDIGMG | 47 |
| CDR-H2 | HIWWNDREYYNSALQS | 48 |
| CDR-H3 | IDYFGSGQAWFTY | 49 |
| CDR-H2 (v5) | HIWWNDREYYSSALQS | 53 |
| CDR-H2 (v6) | HIWWNDREYYNPALQS | 54 |

TABLE 4C
Humanization of 102A10F2-VL

| Name | Sequence | SEQ ID NO: |
|---|---|---|
| 102A10F2 VL | EIVLTQSPPTMAASPGEKITITCSSSSTISSTYLHWYQQKPGFSPKLLIS | 6 |
| | GTSNLASGVPPRESGSGSGTSYSLTIGTLEAEDVATYYCQQGSRIPFTFG | |
| | SGTKLEIK | |
| VI (CDR | EIVLTQSPGTLSLSPGERATLSCSSSSTISSTYLHWYQQKPGQAPRLLIY | 61 |
| grafting) | GTSNLASGIPDRESGSGSGTDETLTISRLEPEDFAVYYCQQGSRIPFTFG | |
| | QGTKLEIK | |
| V2 (with back | EIVLTQSPGTLSLSPGERATLSCSSSSTISSTYLHWYQQKPGQAPRLLIS | 62 |
| mutations) | GTSNLASGIPDRESGSGSGTDYTLTISRLEPEDVAVYYCQQGSRIPETEG | |
| | QGTKLEIK | |

TABLE 4D
CDR Sequences

| CDR | Sequence | SEQ ID NO: |
|---|---|---|
| CDR-L1 | SSSSTISSTYLH | 50 |
| CDR-L2 | GTSNLAS | 51 |
| CDR-L3 | QQGSRIPFT | 52 |

TABLE 3E
Humanized antibodies

| | VL | VL v1 | VL v2 |
|---|---|---|---|
| VH | HSP210-01-Chi | | |
| VH v1 | | HSP210-01-hz11 | HSP210-01-hz12 |
| VH v2 | | HSP210-01-hz21 | HSP210-01-hz22 |
| VH v3 | | HSP210-01-hz31 | HSP210-01-hz32 |
| VH v4 | | HSP210-01-hz41 | HSP210-01-hz42 |
| VH v5 | | HSP210-01-hz51 | HSP210-01-hz52 |
| VH v6 | | HSP210-01-hz61 | HSP210-01-hz62 |

### D. 62D2H6

TABLE 5A
Humanization of 62D2H6-VH

| Name | Sequence | SEQ ID NO: |
|---|---|---|
| 62D2H6 VH | EVQLQQSGAELVKPGASVKLSCTASGFNIKDYYMHWVKQRTEQGLEWIGR | 7 |
| | IDPEDGETKYAPKFQGKATITADTSSNTAYLQLSSLTSEDTAVYYCSRSW | |
| | AYWGQGTTLTVSS | |
| V1 (CDR | EVQLVQSGAEVKKPGATVKISCKVSGENIKDYYMHWVQQAPGKGLEWMGR | 69 |
| grafting) | IDPEDGETKYAPKFQGRVTITADTSTDTAYMELSSLRSEDTAVYYCATSW | |
| | AYWGQGTTVTVSS | |
| V2 (with back | EVQLVQSGAEVKKPGATVKISCKASGENIKDYYMHWVQQAPGKGLEWMGR | 70 |
| mutations) | IDPEDGETKYAPKFQGRVTITADTSTNTAYMELSSLRSEDTAVYYCSRSW | |
| | AYWGQGTTVTVSS | |
| V3 (chimeric | QVQLVQSGAEVKKPGASVKVSCKASGENIKDYYMHWVRQAPGQGLEWMGR | 71 |
| 2) | IDPEDGETKYAPKFQGRVTMTRDTSTSTVYMELSSLRSEDTAVYYCARSW | |
| | AYWGQGTTVTVSS | |
| V4 (with back | QVQLVQSGAEVKKPGASVKVSCKASGFNIKDYYMHWVRQAPGQGLEWMGR | 72 |
| mutations) | IDPEDGETKYAPKFQGRVTMTADTSTNTAYMELSSLRSEDTAVYYCSRSW | |
| | AYWGQGTTVTVSS | |

TABLE 5B
CDR Sequences

| CDR | Sequence | SEQ ID NO: |
|---|---|---|
| CDR-H1 | DYYMH | 63 |
| CDR-H2 | RIDPEDGETKYAPKFQG | 64 |
| CDR-H3 | SWAY | 65 |

TABLE 5C
Humanization of 62D2H6-VL

| Name | Sequence | SEQ ID NO: |
|---|---|---|
| 62D2H6 VL | QIVLTQSPAIMSASPGEKVTLTCSASSSVSSSYLYWYQQKPGSSPKLWIYST | 8 |
| | SNLASGVPARFSGSGSGTSYSLTISSMEAEDAASYFCHQWSSYPRTFGGGTK | |
| | LEIK | |
| V1 (CDR | EIVLTQSPATLSLSPGERATLSCSASSSVSSSYLYWYQQKPGQAPRLLIYST | 73 |
| grafting) | SNLASGIPARFSGSGSGTDETLTISSLEPEDFAVYYCHQWSSYPRTFGGGTK | |
| | VEIK | |
| V2 (with back | EIVLTQSPATLSLSPGERATLSCSASSSVSSSYLYWYQQKPGQAPRLLIYST | 74 |
| mutations) | SNLASGIPARFSGSGSGTDYTLTISSLEPEDAAVYFCHQWSSYPRTFGGGTK | |
| | VEIK | |
| V3 (chimeric | EIVMTQSPPTLSLSPGERVTLSCSASSSVSSSYLYWYQQKPGQAPRLLIYST | 75 |
| 2) | SNLASGIPARFSGSGSGTDETLTISSLQPEDFAVYYCHQWSSYPRTFGGGTK | |
| | VEIK | |
| V4 (with back | EIVMTQSPPTLSLSPGERVTLSCSASSSVSSSYLYWYQQKPGQAPRLLIYST | 76 |
| mutations) | SNLASGIPARFSGSGSGTDYTLTISSLQPEDAAVYFCHQWSSYPRTFGGGTK | |
| | VEIK | |

TABLE 5D
CDR Sequences

| CDR | Sequence | SEQ ID NO: |
|---|---|---|
| CDR-L1 | SASSSVSSSYLY | 66 |
| CDR-L2 | STSNLAS | 67 |
| CDR-L3 | HQWSSYPRT | 68 |

TABLE 5E
Humanized antibodies

| | VL | VL v1 | VL v2 | VL v3 | VL v4 |
|---|---|---|---|---|---|
| VH | Chimeric | | | | |
| VH v1 | | hz11 | | | |
| VH v2 | | | hz22 | | hz24 |
| VH v3 | | | | hz33 | |
| VH v4 | | | hz42 | | hz44 |

### E. 211F8E11

TABLE 6A
Humanization of 211F8E11-VH

| Name | Sequence | SEQ ID NO: |
|---|---|---|
| 211F8E11 | EVQLQQSGAELVKPGASVKLSCTASGENIKDTYMHWVKQRPEQGLEWVGR | 9 |
| VH | IDPANVNTIYDPKFQGKATITADTSSNTAYLQLSSLTSEDTAVYYCARVG | |
| | AYDGYDFDYWGQGTTLTVSS | |
| V1 (CDR | EVQLVQSGAEVKKPGATVKISCKVSGENIKDTYMHWVQQAPGKGLEWMGR | 83 |
| grafting) | IDPANVNTIYDPKFQGRVTITADTSTDTAYMELSSLRSEDTAVYYCATVG | |
| | AYDGYDFDYWGQGTTVTVSS | |
| V2 (with back | EVQLVQSGAEVKKPGATVKISCKASGFNIKDTYMHWVQQAPGKGLEWMGR | 84 |
| mutations) | IDPANVNTIYDPKFQGRVTITADTSTNTAYMELSSLRSEDTAVYYCARVG | |
| | AYDGYDEDYWGQGTTVTVSS | |
| V3 (chimeric | QVQLVQSGAEVKKPGASVKVSCKASGENIKDTYMHWVRQAPGQGLEWMGR | 85 |
| 2) | IDPANVNTIYDPKFQGRVTMTRDTSTSTVYMELSSLRSEDTAVYYCARVG | |
| | AYDGYDEDYWGQGTTVTVSS | |
| V4 (with back | QVQLVQSGAEVKKPGASVKVSCKASGENIKDTYMHWVRQAPGQGLEWMGR | 86 |
| mutations) | IDPANVNTIYDPKFQGRVTMTADTSTNTAYMELSSLRSEDTAVYYCARVG | |
| | AYDGYDFDYWGQGTTVTVSS | |

TABLE 6B
CDR Sequences

| CDR | Sequence | SEQ ID NO: |
|---|---|---|
| CDR-H1 | DTYMH | 77 |
| CDR-H2 | RIDPANVNTIYDPKFQG | 78 |
| CDR-H3 | VGAYDGYDEDY | 79 |

TABLE 6C
Humanization of 211F8E11-VL

| Name | Sequence | SEQ ID NO: |
|---|---|---|
| 211F8E11 | DIVLTQSPASLAVSLGQRATISCRASESVDNYGNSFMHWYQQKPGQPPKLLI | 10 |
| VL | YRASNLESGIPARFSGSGSRTDFTLTINPVEADDVATYYCQQNNEDPLTFGA | |
| | GTKLELK | |
| V1 (CDR | DIVMTQSPDSLAVSLGERATINCRASESVDNYGNSFMHWYQQKPGQPPKLLI | 87 |
| grafting) | YRASNLESGVPDRESGSGSGTDETLTISSLQAEDVAVYYCQQNNEDPLTFGQ | |
| | GTKLEIK | |
| V2 (with back | DIVLTQSPDSLAVSLGERATINCRASESVDNYGNSFMHWYQQKPGQPPKLLI | 88 |
| mutations) | YRASNLESGVPDRESGSGSRTDETLTISSLQAEDVAVYYCQQNNEDPLTFGQ | |
| | GTKLEIK | |
| V3 (chimeric | DIQMTQSPSSLSASVGDRVTITCRASESVDNYGNSFMHWYQQKPGKVPKLLI | 89 |
| 2) | YRASNLESGVPSRESGSGSGTDETLTISSLQPEDVATYYCQQNNEDPLTFGQ | |
| | GTKLEIK | |
| V4 (with back | DIQLTQSPSSLSASVGDRVTITCRASESVDNYGNSFMHWYQQKPGKVPKLLI | 90 |
| mutations) | YRASNLESGVPSRESGSGSRTDETLTISSLQPEDVATYYCQQNNEDPLTFGQ | |
| | GTKLEIK | |

TABLE 6D
CDR Sequences

| CDR | Sequence | SEQ ID NO: |
|---|---|---|
| CDR-L1 | RASESVDNYGNSFMH | 80 |
| CDR-L2 | RASNLES | 81 |
| CDR-L3 | QQNNEDPLT | 82 |

TABLE 6E
Humanized antibodies

| | VL | VL v1 | VL v2 | VL v3 | VL v4 |
|---|---|---|---|---|---|
| VH | Chimeric | | | | |
| VH v1 | | hz11 | | | |
| VH v2 | | | hz22 | | hz24 |
| VH v3 | | | | hz33 | |
| VH v4 | | | hz42 | | hz44 |

### F. 217D11E5

TABLE 7A
Humanization of 217D11E5-VH

| Name | Sequence | SEQ ID NO: |
|---|---|---|
| 217D11E5 | EVQLQQSGPELVKPGASVKMSCKASGYTFTSYVMHWVKQKPGQGLEWIGY | 11 |
| VH | INPYNDGTKYNEKFKGKATLTSDKSSSTAYMELSSLTSEDSAVYYCARSY | |
| | YDYDGSFDYWGQGTTLTVSS | |
| V1 (CDR | QVQLVQSGAEVKKPGASVKVSCKASGYTFTSYVMHWVRQAPGQRLEWMGY | 97 |
| grafting) | INPYNDGTKYNEKFKGRVTITRDTSASTAYMELSSLRSEDTAVYYCARSY | |
| | YDYDGSFDYWGQGTTVTVSS | |
| V2 (with back | QVQLVQSGAEVKKPGASVKVSCKASGYTFTSYVMHWVRQAPGQRLEWMGY | 98 |
| mutations) | INPYNDGTKYNEKFKGRVTITSDKSASTAYMELSSLRSEDTAVYYCARSY | |
| | YDYDGSFDYWGQGTTVTVSS | |
| V3 (chimeric | QVQLVQSGAEVKKPGASVKVSCKASGYTFTSYVMHWVRQAPGQGLEWMGY | 99 |
| 2) | INPYNDGTKYNEKFKGRVTMTRDTSTSTVYMELSSLRSEDTAVYYCARSY | |
| | YDYDGSFDYWGQGTTVTVSS | |
| V4 (with back | QVQLVQSGAEVKKPGASVKVSCKASGYTFTSYVMHWVRQAPGQGLEWMGY | 100 |
| mutations) | INPYNDGTKYNEKFKGRVTMTSDKSTSTAYMELSSLRSEDTAVYYCARSY | |
| | YDYDGSFDYWGQGTTVTVSS | |

TABLE 7B
CDR Sequences

| CDR | Sequence | SEQ ID NO: |
|---|---|---|
| CDR-H1 | SYVMH | 91 |
| CDR-H2 | YINPYNDGTKYNEKFKG | 92 |
| CDR-H3 | SYYDYDGSFDY | 93 |

TABLE 7C
Humanization of 217D11E5 - VL

| Name | Sequence | SEQ ID NO: |
|---|---|---|
| 217D11E5 | DIVMTQSHKEMSTSVGDRVSITCKASQDVTTAVAWYQQKSGQSPKLLIYSAS | 12 |
| VL | YRYTGDPDRFTGSGSGTDFTFTISSVQAEDLAVYYCQQHYSTPWTFGGGTKL | |
| | EIK | |
| V1 (CDR | DIQMTQSPSSLSASVGDRVTITCKASQDVTTAVAWYQQKPGKAPKLLIYSAS | 101 |
| grafting) | YRYTGVPSRFSGSGSGTDETFTISSLQPEDIATYYCQQHYSTPWTFGGGTKV | |
| | EIK | |
| V2 (chimeric | DIQMTQSPSSLSASVGDRVTITCKASQDVTTAVAWYQQKPGKVPKLLIYSAS | 102 |
| 2) | YRYTGVPSRFSGSGSGTDETLTISSLQPEDVATYYCQQHYSTPWTFGGGTKV | |
| | EIK | |

TABLE 7D
CDR Sequences

| CDR | Sequence | SEQ ID NO: |
|---|---|---|
| CDR-L1 | KASQDVITAVA | 94 |
| CDR-L2 | SASYRYT | 95 |
| CDR-L3 | QQHYSTPWT | 96 |

TABLE 7E
Humanized antibodies

| | VL | VL v1 | VL v2 |
|---|---|---|---|
| VH | Chimeric | | |
| VH v1 | | hz11 | |
| VH v2 | | hz21 | hz22 |
| VH v3 | | | |
| VH v4 | | hz41 | hz42 |

### G. 234B7D5

TABLE 8A
Humanization of 234B7D5-VH

| Name | Sequence | SEQ ID NO: |
|---|---|---|
| 234B7D5 | EVQLQQSGAELVKPGASVKLSCTASGENFEDTYIHWVKQRPDQGLEWIGRID | 13 |
| VH | PADGDTKHNPKFHDKATVTVDTSSNTAYLELSSLISEDTAVYYCVRGNYVNW | |
| | GQGTTLTVSS | |
| V1 (CDR | EVQLVQSGAEVKKPGATVKISCKVSGENFEDTYIHWVQQAPGKGLEWMGRID | 109 |
| grafting) | PADGDTKHNPKFHDRVTITADTSTDTAYMELSSLRSEDTAVYYCATGNYVNW | |
| | GQGTTVTVSS | |
| V2 (with back | EVQLVQSGAEVKKPGATVKISCKASGENFEDTYIHWVQQAPGKGLEWMGRID | 110 |
| mutations) | PADGDTKHNPKFHDRVTITVDISTINTAYMELSSLRSEDTAVYYCVRGNYVNW | |
| | GQGTTVTVSS | |
| V3 (chimeric | QVQLVQSGAEVKKPGASVKVSCKASGENFEDTYIHWVRQAPGQGLEWMGRID | 111 |
| 2) | PADGDTKHNPKFHDRVTMTRDTSTSTVYMELSSLRSEDTAVYYCARGNYVNW | |
| | GQGTTVTVSS | |
| V4 (with back | QVQLVQSGAEVKKPGASVKVSCKASGENFEDTYIHWVRQAPGQGLEWMGRID | 112 |
| mutations) | PADGDTKHNPKFHDRVTMTVDTSTNTAYMELSSLRSEDTAVYYCVRGNYVNW | |
| | GQGTTVTVSS | |

TABLE 8B
CDR Sequences

| CDR | Sequence | SEQ ID NO: |
|---|---|---|
| CDR-H1 | DTYIH | 103 |
| CDR-H2 | RIDPADGDTKHNPKFHD | 104 |
| CDR-H3 | GNYVN | 105 |

TABLE 7C
Humanization of 234B7D5-VL

| Name | Sequence | SEQ ID NO: |
|---|---|---|
| 234B7D5 VL | QIVLIQSPAIMSASPGERVTLTCRASSSVTSSYLYWYQQKPGSSPKLWIYSA | 14 |
| | SNLASGVPARFSGSGSGTSYSLTISSVEAEDAASYFCHQWYSYPRTFGGGTK | |
| | LEIK | |
| V1 (CDR | EIVLTQSPATLSLSPGERATLSCRASSSVTSSYLYWYQQKPGQAPRLLIYSA | 113 |
| grafting) | SNLASGIPARFSGSGSGTDFTLTISSLEPEDFAVYYCHQWYSYPRTFGGGTK | |
| | VEIK | |
| V2 (with back | EIVLTQSPATLSLSPGERATLSCRASSSVTSSYLYWYQQKPGQAPRLLIYSA | 114 |
| mutations) | SNLASGIPARFSGSGSGTDYTLTISSLEPEDAAVYFCHQWYSYPRTFGGGTK | |
| | VEIK | |
| V3 (chimeric | EIVLTQSPGTLSLSPGERATLSCRASSSVTSSYLYWYQQKPGQAPRLLIYSA | 115 |
| 2) | SNLASGIPDRESGSGSGTDETLTISRLEPEDFAVYYCHQWYSYPRTEGGGTK | |
| | VEIK | |
| V4 (with back | EIVLTQSPGILSLSPGERATLSCRASSSVTSSYLYWYQQKPGQAPRLLIYSA | 116 |
| mutations) | SNLASGIPDRESGSGSGTDYTLTISRLEPEDAAVYFCHQWYSYPRTFGGGTK | |
| | VEIK | |
| V5 (chimeric | EIVMTQSPPTLSLSPGERVTLSCRASSSVTSSYLYWYQQKPGQAPRLLIYSA | 117 |
| 3) | SNLASGIPARFSGSGSGTDETLTISSLQPEDFAVYYCHQWYSYPRTFGGGTK | |
| | VEIK | |
| V6 (with back | EIVMTQSPPTLSLSPGERVTLSCRASSSVTSSYLYWYQQKPGQAPRLLIYSA | 118 |
| mutations) | SNLASGIPARFSGSGSGTDYTLTISSLQPEDAAVYFCHQWYSYPRTFGGGTK | |
| | VEIK | |

TABLE 8D
CDR Sequences

| CDR | Sequence | SEQ ID NO: |
|---|---|---|
| CDR-L1 | RASSSVTSSYLY | 106 |
| CDR-L2 | SASNLAS | 107 |
| CDR-L3 | HQWYSYPRT | 108 |

TABLE 8E
Humanized antibodies

| | | | | | | VL | |
|---|---|---|---|---|---|---|---|
| | VL | VL v1 | VL v2 | VL v3 | VL v4 | v5 | VL v6 |
| VH | Chimeric | | | | | | |
| VH v1 | | hz11 | | | | | |
| VH v2 | | | hz22 | | hz24 | | hz26 |
| VH v3 | | | | hz33 | | | |
| VH v4 | | | hz42 | | hz44 | | hz46 |

### Example 6

### Testing of Humanized Antibodies

This example tested some of the humanized antibodies for the ability to block interactions between SIRPα and CD47.

Recombinant CD47-Fc fusion protein (Acrobiosystems) was coated at 1 μg/ml in PBS onto microtiter plates for 16 hours at 4° C. After blocking for 1 h with 1% BSA in PBST at RT, 1 μg/mL of SIRPα-His protein was added either in the absence or presence of different concentrations of the anti-SIRPα antibodies at RT for 1 h. Plates were subsequently washed three times and incubated with an HRP-conjugated anti-His secondary antibody for 1 h at RT. After washing, the TMB solution was added to each well for 30 min and the reaction was stopped with 2M H₂SO₄, and OD was measured at 490 nm.

All of the antibodies listed in Tables 2E (248G3F6), 3E (300A6A6), and 4E (102A10F2) were tested and exhibited high IC50 (Table 9).

TABLE 9
Activities of humanized antibodies
to block SIRPα interaction with CD47

| | Antibody | IC₅₀ (nM) |
|---|---|---|
| 248G3F6 | 02-chi | 0.14 |
| | 02-hz22 | 0.092 |
| | 02-hz32 | 0.11 |
| | 02-hz42 | 0.12 |
| | 02-hz52 | 0.11 |
| 300A6A6 | 03-chi | 0.14 |
| | 03-hz22 | 0.16 |
| | 03-hz32 | 0.145 |
| | 03-hz42 | 0.13 |
| | 03-hz52 | 0.13 |
| 102A10F2 | 01-hz22 | 0.21 |
| | 01-hz32 | 0.16 |
| | 01-hz52 | 0.23 |
| | 01-hz61 | 0.21 |
| | 01-hz62 | 0.16 |

### Example 7

### Increase of Macrophage Mediated Phagocytosis of Tumor Cells

This example tested some of the humanized antibodies for their ability to increase macrophage mediated phagocytosis of tumor cells.

PBMCs were isolated from human blood, and monocytes were differentiated into macrophages using a standard protocol. The monocyte derived macrophages (MDMs) were scraped and re-plated in 24-well dishes and allowed to adhere for 24 hrs. The human tumor cell line Raji that endogenously expressed CD47 were selected as target cells and labeled with 1 uM CFSE for 10 mins, then added to MDMs at a ratio of 5:1 tumor cells per phagocyte and different concentrations of anti-SIRPα antibodies was added at the indicated concentrations. After 3 hr incubation, non-phagocytosed target cells were washed away with PBS and the remaining phagocytes were scraped off, stained with CD14 antibody, and analyzed by flow cytometry. Phagocytosis was measured by gating on CD14⁺ cells and then assessing the percentage of CFSE⁺ cells.

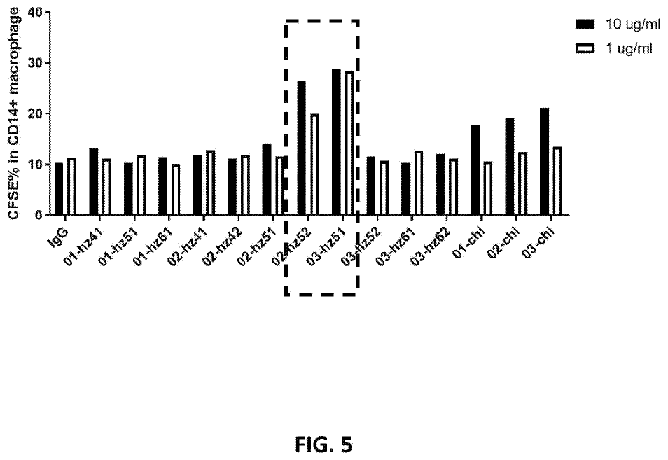

The results are presented in FIG. 5. Out of the tested antibodies, 02-hz52 (248G3F6) and 03-hz51 (300A6A6) exhibited the highest activities, and all others showed excellent activities as well.

### Example 8

### Binding Affinity to SIRPα v1 and v2

Humanized antibodies 02-hz52 (248G3F6) and 03-hz51 (300A6A6) were tested for their binding affinities to SIRPα v1 and v2 in this example.

The binding kinetics assay of antibody to antigen was performed using Biacore 8K system through human antibody capture approach. The anti-mouse Fc lgG were immobilized on CM5 sensor chip according to the manufactory's instruction. The test antibody was injected and captured by the immobilized anti-human Fc lgG. And then serial concentrations of human SIRPα v1 or SIRPα v2 protein were individually injected, and the binding profile was recorded for each concentration antigen analyte, respectively. The assay system was regenerated by injection of 10 mM Glycine-HCl pH 1.5 for 30 seconds. The running buffer was HBS-EP+ (10 mM HEPES, pH 7.4, 150 mM NaCl, 3 mM EDTA and 0.05% P20). The assay temperature was 25° C., and the association and dissociation time were 180 and 600 seconds, respectively. The Biacore data were fitted using Biacore K8 evaluation software 1.0 according to 1:1 binding model to calculate the association (ka) and dissociation (kd) rate constants as well as the equilibrium constant (KD).

The testing results are shown in Table 10A-B.

TABLE 10A
Binding affinity against SIRPα v1

| Sample | ka (1/Ms) | kd (1/s) | KD (M) |
|---|---|---|---|
| 02-hz52 | 7.04E+05 | 2.98E−04 | 4.23E−10 |
| 03-hz51 | 8.05E+05 | 8.04E−04 | 9.99E−10 |

TABLE 10B
Binding affinity against SIRPα v2

| Sample | ka (1/Ms) | kd (1/s) | KD (M) |
|---|---|---|---|
| 02-hz52 | 3.33E+05 | 2.47E−03 | 7.40E−09 |
| 03-hz51 | 3.31E+05 | 1.00E−03 | 3.03E−09 |

### Example 9

### Generation of Anti-PD-L1 Single Domain Antibodies (sdAbs)

### Immunization

Two llamas were immunized with recombinant PD-L1 ECD protein under all current animal welfare regulations. For immunization, the antigen was formulated as an emulsion with CFA (primary immunization) or IFA (boost immunization). The antigen was administered by double-spot injections intramuscularly at the neck. Each animal received two injections of the emulsion, containing 100 μg of PD-L1 ECD and 4 subsequent injections containing 50 μg of antigen at weekly intervals. At different time points during immunization, 10 ml blood samples were collected from the animal and sera were prepared. The induction of an antigen specific humoral immune response was verified using the serum samples in an ELISA-based experiment with immobilized PD-L1 ECD protein. Five days after the last immunization, a blood sample of 300 ml was collected. Peripheral blood lymphocytes (PBLs), as the genetic source of the llama heavy chain immunoglobulins (HCAbs), were isolated from the 300 ml blood sample using a Ficoll-Paque gradient (Amersham Biosciences), yielding 1×10⁹ PBLs. The maximal diversity of antibodies is expected to be equal to the number of sampled B-lymphocytes, which is about 10% of the number of PBLs (1×10⁸). The fraction of heavy-chain antibodies in llama is up to 20% of the number of B-lymphocytes. Therefore, the maximal diversity of HCAbs in the 300 ml blood sample is calculated as 2×10⁷ different molecules.

### Library Construction

RNA extracted from PBLs and lymph node was used as starting material for RT-PCR to amplify sdAb encoding gene fragments. These fragments were cloned into an in-house phagemid vector. In frame with the sdAb coding sequence, the vector coded for a C-terminal (His)6 tag. The library size is more than 1×10⁹. The library phage was prepared according to a standard protocol and stored after filter sterilization at 4° C. for further use.

### Selections and High-Throughput Screening

Selections were carried out with the above libraries using solid panning as well as cell-based panning. Only a single round of selection was performed for both conditions. Each selection output was analyzed for enrichment factor (# phage present in eluate relative to control), diversity and percentage of PD-L1 positive clones (ELISA). Based on these parameters the best selections were chosen for further analysis. To this end, the output from each selection was recloned as a pool into a soluble expression vector for high-throughput screening. In frame with the sdAb coding sequence, the vector coded for a C-terminal (His)6 tag. Colonies were picked and grown in 96 deep well plates (1 ml volume) and induced by adding IPTG and 0.1% Triton for sdAb expression in the supernatant.

The supernatant was analyzed for their ability to bind to PD-L1 ECD protein (by ELISA) and PD-L1 stable cell line (by FACS). The positive binders were sequenced and the unique clones were selected for further characterization.

The unique clones were grown in 2XYT medium and induced by IPTG for sdAb expression in the supernatant. The supernatant of unique binders were analyzed for their ability to inhibit PD-L1-PD-1 interaction. To this end, the supernatant was incubated with PD-L1 ECD protein, then the complex was added to PD-1 stable cell line for binding evaluation. sdAbs with negative signal on PD-1 cell line are considered as PD-L1 inhibitors.

All potential inhibitors were selected for off-rate analysis by surface plasmon resonance (SPR) on a BIAcore T200 instrument. The dissociation phase was used to calculate the k_{off} values for each individual sdAb.

### sdAb Production

The His6-tagged sdAbs were purified from periplasmic extracts by ÄKTA. The NTA resin was processed according to the manufacturer's instructions. Periplasmic extracts prepared were incubated with the resin for 30 min at RT on a rotator. The resin was washed with PBS and transferred to a column. The packed resin was washed with 15 mM Imidazole. sdAbs were eluted from the column using 150 mM Imidazole. The eluted fractions were analyzed by spotting on Hybond Membrane and visualized with Ponceau. Fractions containing protein were pooled and dialyzed against PBS. Dialyzed protein was collected, filter sterilized, concentration determined and stored at −20° C.

To determine the purity, protein samples were analyzed on a 12% SDS-PAGE gel. 10 Laemmli sample buffer was added to 10 μl (2 μg) purified protein, then the sample was heated for 10 minutes at 95° C., cooled and loaded onto a 12% SDS-PAGE gel. The gel was processed according to general procedures and stained with Coomassie Brilliant Blue (CBB).

### HCAb Production

Heavy chain-only antibody (HCAb) constructs were generated by fusing sdAbs with human Fc region. The maxiprep of the HCAb constructs were prepared for CHO-K1 cell transient expression and purification. The expressed HCAbs were purified by chromatography through a column containing Protein A agarose resin followed by a size exclusion column.

To determine the purity, protein samples were analyzed on a 12% SDS-PAGE gel. 10 Laemmli sample buffer was added to 10 μl (2 μg) purified protein, then the sample was heated for 10 minutes at 95° C., cooled and loaded onto a 12% SDS-PAGE gel. The gel was processed according to general procedures and stained with Coomassie Brilliant Blue (CBB). The purity of purified HCAbs are >85%. The data were summarized in Table 11.

TABLE 11
Summary of HCAb purification

| Sample | AS06617 | AS06618 | AS06628 | AS06682 | AS06686 |
|---|---|---|---|---|---|
| Conc.(mg/ml) | 1.50 | 1.70 | 1.58 | 1.55 | 1.48 |
| Amount(mg) | 10.48 | 20.42 | 16.60 | 16.26 | 14.83 |
| Purity | >85% | >85% | >85% | >85% | >85% |
| Endotoxin level(EU/μg) | <0.01 | <0.01 | <0.01 | <0.01 | <0.01 |
| Sample | AS06703 | AS06730 | AS06750 | AS06775 | AS06778 |
| Conc.(mg/ml) | 1.35 | 1.81 | 1.81 | 1.63 | 1.41 |
| Amount(mg) | 13.45 | 21.77 | 21.70 | 19.51 | 12.71 |
| Purity | >85% | >85% | >85% | >85% | >85% |
| Endotoxin level(EU/μg) | <0.01 | <0.01 | <0.01 | <0.01 | <0.01 |

| Sample | AS06791 | AS11947 | AS11948 | AS12003 |
|---|---|---|---|---|
| Conc.(mg/ml) | 1.82 | 1.80 | 1.58 | 2.02 |
| Amount(mg) | 21.82 | 19.81 | 17.38 | 24.25 |
| Purity | >85% | >85% | >85% | >85% |
| Endotoxin level(EU/μg) | <0.01 | 0.01~0.1 | <0.01 | <0.01 |

### sdAb Affinity Determination and HCAb Affinity Determination

Affinity constant (K_{d}) of each sdAb and HCAb was determined by surface plasmon resonance (SPR) on a BIAcore T200 instrument. Briefly, PD-L1 His was amine-coupled to a CMS sensor chip at a density of no higher than 100 RU. Anti-PD-L1 sdAbs or anti-PD-L1 HCAbs were injected at 5 different concentrations between 0.33 and 27 nM. Flow rate was 30 μl/min in all experiments. Association and dissociation phases were 5 and 10 min, respectively. The chip was regenerated using Glycine/HCl pH 1.5. Binding curves at different concentrations of sdAbs and HCAbs were used to calculate the kinetic parameters kₒₙ, k_{off} and K_{D}. The kinetics data were summarized in Table 12 and Table 13.

TABLE 12
affinity determination of sdAbs against PD-L1

| Ligand | Analyte | kₐ (1/Ms) | k_{d} (1/s) | K_{D} (M) |
|---|---|---|---|---|
| PD-L1/His | AS06617 sdAb | 1.86E+06 | 4.64E−04 | 2.49E−10 |
| | AS06618 sdAb | 1.44E+06 | 2.45E−04 | 1.70E−10 |
| | AS06628 sdAb | 4.10E+06 | 2.15E−04 | 5.26E−11 |
| | AS06682 sdAb | 1.68E+06 | 3.42E−04 | 2.03E−10 |
| | AS06686 sdAb | 2.79E+06 | 9.65E−04 | 3.46E−10 |
| | AS06703 sdAb | 1.80E+06 | 6.96E−05 | 3.87E−11 |
| | AS06730 sdAb | 7.11E+06 | 1.39E−04 | 1.95E−11 |
| | AS06750 sdAb | 2.05E+06 | 2.23E−04 | 1.09E−10 |
| | AS06775 sdAb | 1.58E+06 | 2.47E−04 | 1.56E−10 |
| | AS06778 sdAb | 1.90E+06 | 4.42E−05 | 2.33E−11 |
| | AS06791 sdAb | 1.56E+06 | 2.39E−04 | 1.53E−10 |
| | AS11947 sdAb | 1.92E+06 | 1.18E−03 | 6.17E−10 |
| | AS11948 sdAb | 2.37E+06 | 3.94E−04 | 1.67E−10 |
| | AS12003 sdAb | 2.76E+06 | 1.55E−03 | 5.60E−10 |

TABLE 13
affinity determination of HCAbs against PD-L1

| | | kₐ | k_{d} | K_{D} |
|---|---|---|---|---|
| Ligand | Analyte | (1/Ms) | (1/s) | (M) |
| PD-L1/His | AS06617 | 5.20E+05 | 5.51E−05 | 1.06E−10 |
| | HCAb | | | |
| | AS06618 | 4.21E+05 | 1.41E−05 | 3.35E−11 |
| | HCAb | | | |
| | AS06628 | 1.27E+06 | 1.98E−05 | 1.55E−11 |
| | HCAb | | | |
| | AS06730 | 1.52E+06 | 2.44E−05 | 1.61E−11 |
| | HCAb | | | |
| | AS06682 | 5.99E+05 | 3.34E−05 | 5.57E−11 |
| | HCAb | | | |
| | AS06686 | 9.79E+05 | 1.53E−04 | 1.56E−10 |
| | HCAb | | | |
| | AS06703 | 5.55E+05 | <1.00E−05 * | <1.80E−11 * |
| | HCAb | | | |
| | AS06750 | 7.19E+05 | 2.81E−05 | 3.92E−11 |
| | HCAb | | | |
| | AS06775 | 4.76E+05 | 3.29E−05 | 6.90E−11 |
| | HCAb | | | |
| | AS06778 | 5.53E+05 | <1.00E−05 * | <1.81E−11 * |
| | HCAb | | | |
| | AS06791 | 7.81E+05 | 5.33E−05 | 6.83E−11 |
| | HCAb | | | |
| | AS11947 | 7.30E+05 | 2.26E−04 | 3.10E−10 |
| | HCAb | | | |
| | AS11948 | 7.61E+05 | 8.25E−05 | 1.08E−10 |
| | HCAb | | | |
| | AS12003 | 5.69E+05 | 2.47E−04 | 4.34E−10 |
| | HCAb | | | |

* kd is outside the limits that can be measured by Biacore T200

### Target Binding Assays

The ability of the purified antigen binding proteins to bind PD-L1 was determined using Surface Plasmon Resonance method (e.g., BIACORE®), an enzyme-linked immunosorbent assay, a Fluorescence-Assisted Cell Sorting method (FACS), or a combination thereof. The analyses can be performed on PD-L1 transfected cells.

CHO-K1 cells expressing human PD-L1 were dissociated from adherent culture flasks and mixed with varying concentrations of antibodies and a constant concentration of anti-PD-L1 sdAbs or HCAbs (in a 96-well plate). Tecentriq® was used as an anti-PD-L1 antibody positive control. The antibody and cell incubation was equilibrated for 30 minutes at room temperature, washed three times with FACS buffer (PBS containing 1% BSA). FITC conjugated anti-human IgG secondary antibody was then added and incubated for 15 minutes at room temperature. Cells were washed again with FACS buffer and analyzed by flow cytometry. Data were analyzed with Prism (GraphPad Software, San Diego, CA) using non-linear regression, and EC₅₀ values were calculated.

### Inhibition of Ligand Binding by FACS Analysis

Blockade of ligand binding was studied using flow cytometry. For anti-PD-L1 HCAbs evaluation, CHO-K1 cells expressing human PD-L1 were dissociated from adherent culture flasks and mixed with varying concentrations of antibodies and a constant concentration of biotin-labeled hPD-1/Fc protein (both in a 96-well plate). Tecentriq® was used as an anti-PD-L1 antibody positive control. The mixture was equilibrated for 30 minutes at room temperature, washed three times with FACS buffer (PBS containing 1% BSA). PE/Cy5 Streptavidin secondary antibody was then added and incubated for 15 minutes at room temperature. Cells were washed again with FACS buffer and analyzed by flow cytometry. Data were analyzed with Prism (GraphPad Software, San Diego, CA) using non-linear regression, and IC₅₀ values were calculated. The competition assays demonstrated the ability of most anti-PD-L1 HCAbs in efficiently inhibiting PD-L1-PD-1 interactions at low concentrations (1-10 μg/ml), the IC₅₀ of most HCAbs are comparable to Tecentriq®.

### PD-L1 -Based Blockade Assay

CHO-K1 stable expressing PD-L1 cells and Jurkat effector cells are used to assess PD-1 blockade for anti-PD-L1 sdAbs and HCAbs evaluation. The effector cells contain a luciferase construct that is induced upon disruption of the PD-1/PD-L1 receptor-ligand interaction, such as when the PD-L1 cells are mixed with effector cells expressing PD-1. Thus, efficacy of inhibiting PD-L1 on CHO-K1 stable cells by anti-PD-L1 sdAbs and HCAbs can be assessed by measuring luciferase reporter activity. The assay is performed as follows.

On day one, PD-L1 cells are thawed in a 37° C. water bath until cells are just thawed (about 3-4 minutes), and 0.5 mL of thawed cells is transferred to 14.5 mL cell recovery medium (10% FBS/F-12). The cell suspension is mixed well by gently inverting the tube 1-2 times. The cell suspension is then transferred to a sterile reagent reservoir, and dispensed into assay plates with 25 μL of cell suspension per well. 100 μL of assay medium is added per well as blank control. 100 μL of cell recovery medium is added per well for wells serving as blank control. The plates are then lidded and incubated overnight in a CO₂ incubator at 37° C.

On the day of assay, fresh assay buffer (RPMI 1640+1% FBS) is prepared. An eight-point serial dilution is performed in assay buffer for each of the control anti-PD-L1 antibody (e.g., Tecentriq®), sdAbs or HCAbs. The starting concentration and dilution scheme is optimized to achieve full dose-response curves. The assay plates containing PD-L1 cells are retrieved from the CO₂ incubator. 95 μl of medium is removed per well from all the wells. 40 μL of serial dilutions of the control anti-PD-L1 antibody, or the antigen binding protein, is added per well to wells containing PD-L1 cells. 80 μL assay buffer is added per well to the blank control wells for each plate.

Next, PD-1 effector Cells are thawed in a 37° C. water bath until cells are just thawed (about 3-4 minutes). The cell suspension is gently mixed in the vial by pipetting up and down, and 0.5 mL of the cells is added to 5.9 mL assay buffer. The cell suspension is mixed well by gently inverting the tube 1-2 times. The cell suspension is then transferred to a sterile reagent reservoir, and 40 μL of the cell suspension is dispensed to each well containing the PD-1 cells and control antibody or bispecific antigen binding protein. The plates are lidded and incubated for six hours at 37° C. in a CO₂ incubator.

The Luciferase Assay System is reconstituted by transferring one bottle of Buffer to the bottle containing Substrate. The system is stored at room temperature and shielded from light for same day use. After 6 hours induction, assay plates are removed from the CO₂ incubator and equilibrated at ambient temperature for 5-10 min. 80 μL of reagent is added to each well. The plates are incubated for 5-10 min at ambient temperature. Luminescence is measured in GloMax® Discover System (Promega, Madison, WI) or a plate reader with glow-type luminescence reading capabilities.

Luminescence is expressed as Relative Light Unit (RLU). The RLU values of wells having diluted antibody or bispecific antigen binding protein is normalized to the RLU of no antibody or bispecific antigen binding protein control to provide Fold of Luciferase Induction. Data is graphed as RLU versus Log₁₀ of concentration of antibody or bispecific antigen binding protein and as Fold of Induction versus Log₁₀ concentration of antibody or bispecific antigen binding protein. The data is fitted to a curve and EC₅₀ of each bispecific antigen binding proteins and the control anti-PD-1 antibody is determined using curve fitting software such as GraphPad Prism (Tables 14 and 15).

TABLE 14
EC₅₀ of PD-L1- based blockade assay for sdAbs

| Sample | AS06617 sdAb | AS06618 sdAb | AS06628 sdAb | AS06682 sdAb | AS06686 sdAb |
|---|---|---|---|---|---|
| EC₅₀ (M) | 6.38E−08 | 4.45E−08 | 2.42E−08 | 4.20E−08 | 3.52E−07 |
| Sample | AS06703 sdAb | AS06730 sdAb | AS06750 sdAb | AS06775 sdAb | AS06778 sdAb |
| EC₅₀ (M) | 1.50E−08 | 1.72E−08 | 3.36E−08 | 7.55E−08 | 2.01E−08 |

| Sample | AS06791 sdAb | AS11947 sdAb | AS11948 sdAb | AS12003 sdAb | Tecentriq |
|---|---|---|---|---|---|
| EC₅₀ (M) | 3.65E−08 | 7.72E−08 | 2.52E−08 | 9.62E−08 | 5.92E−09 |

TABLE 15
EC₅₀ of PD-L1- based blockade assay for HCAbs

| Sample | AS06617 HCAb | AS06618 HCAb | AS06628 HCAb | AS06682 HCAb | AS06686 HCAb |
|---|---|---|---|---|---|
| EC₅₀ (M) | 6.20E−09 | 6.45E−09 | 5.88E−09 | 6.66E−09 | 7.37E−09 |
| Sample | AS06703 HCAb | AS06730 HCAb | AS06750 HCAb | AS06775 HCAb | AS06778 HCAb |
| EC₅₀ (M) | 5.59E−09 | 6.791E−09 | 9.98E−09 | 5.8E−09 | 5.54E−09 |

| Sample | AS06791 HCAb | AS11947 HCAb | AS11948 HCAb | AS12003 HCAb | Tecentriq |
|---|---|---|---|---|---|
| EC₅₀ (M) | 5.82E−09 | 7.55E−09 | 5.80E−09 | 9.34E−09 | 5.92E−09 |

### Example 10

### Anti-PD-L1 sdAb Humanization

Five anti-PD-L1 sdAbs (AS06730, AS06750, AS11948, AS06617 and AS06675) were selected for humanization. Protein sequences of wildtype camelid sdAb was aligned with the 5 closest human germline sequences sharing the highest degree of homology. The best human germline sequence was selected as human acceptor. Homology model was made. According to the model analysis data, residues potentially critical for antigen binding or antibody scaffold formation were left untouched while the rest were selected for conversion into the human counterpart. Initially a panel of four sequence optimized variants was generated (stage 1). These variants were analyzed for a number of parameters and the results obtained were used to design a second set of sdAbs (stage 2). For each wildtype sdAb, 1-9 humanized sdAbs were designed for binding, stability and functional evaluation.

### Humanized HCAb Production

The HCAb constructs were generated by fusing sdAbs with the human Fc region. The maxiprep of the HCAb constructs were prepared for CHO-K1 cell transient expression and purification. The expressed HCAbs were purified by chromatography through a column containing Protein A agarose resin followed by a size exclusion column.

### Affinity Ranking of Humanized HCAbs

Binding kinetics of each humanized HCAb to PD-L1 are determined using recombinant human PD-L1 His protein (R&D System) coated on a CM5 (Biacore) sensor chip. Each antigen binding protein is flowed over the antigen-coated chip, using surface plasmon resonance. Alternatively, each antigen binding protein is captured on a CM5 sensor chip, over which human PD-1-His protein is applied. Only the binding affinity of humanized clones comparable to that of the parent HCAbs were selected for further characterization (Tables 16-20).

TABLE 16
Affinity ranking of humanized sdAbs (AS06730)

| Ligand | Analyte | kₐ (1/Ms) | k_{d} (1/s) | K_{D} (M) |
|---|---|---|---|---|
| PD-L1/His | AS06730 | 2.34E+05 | 3.35E−04 | 1.43E−09 |
| | AS06730S | 2.29E+05 | 5.73E−04 | 2.51E−09 |
| | AS06730A | 6.96E+05 | 2.41E−02 | 3.46E−08 |
| | AS06730Q | 3.68E+11 | 1.18E+03 | 3.21E−09 |
| | AS06730QVH1 | 2.58E+06 | 5.79E−03 | 2.24E−09 |
| | AS06730QVH2 | 5.30E+05 | 2.19E−03 | 4.14E−09 |
| | AS06730QVH3a | 2.32E+06 | 2.12E−01 | 9.14E−08 |
| | AS06730SVH12 | 3.0E+05 | 1.8E−03 | 6.1E−09 |
| | AS06730SVH12M8 | 3.1E+05 | 6.5E−03 | 2.1E−08 |
| | AS06730SVH12M9 | 3.2E+05 | 1.2E−02 | 3.7E−08 |

TABLE 17
Affinity ranking of humanized sdAbs (AS06750)

| Ligand | Analyte | kₐ (1/Ms) | k_{d} (1/s) | K_{D} (M) |
|---|---|---|---|---|
| PD-L1/His | AS06750 | 1.49E+05 | 3.30E−04 | 2.21E−09 |
| | AS06750VH2 | 2.12E+05 | 3.29E−04 | 1.55E−09 |
| | AS06750VH3 | 2.02E+05 | 3.55E−04 | 1.75E−09 |
| | AS06750VHa | 1.89E+05 | 3.08E−04 | 1.63E−09 |
| | AS06750VH1 | 7.08E+04 | 2.53E−03 | 3.57E−08 |
| | AS06750VH11 | 1.40E+05 | 2.70E−04 | 1.90E−09 |

TABLE 18
Affinity ranking of humanized sdAbs (AS11948)

| Ligand | Analyte | kₐ (1/Ms) | k_{d} (1/s) | K_{D} (M) |
|---|---|---|---|---|
| PD-L1/His | AS11948 | 2.97E+05 | 5.82E−04 | 1.96E−09 |
| | AS11948Q | 3.01E+05 | 4.65E−02 | 1.55E−07 |
| | AS11948QVH1 | 3.32E+05 | 7.99E−04 | 2.41E−09 |
| | AS11948QVH2 | 2.17E+06 | 1.95E−01 | 8.98E−08 |
| | AS11948A | 2.05E+06 | 1.96E−01 | 9.55E−08 |
| | AS11948QVHa | 6.41E+10 | 6.77E+02 | 1.06E−08 |
| | AS11948S | 2.26E+05 | 8.61E−04 | 3.80E−09 |
| | AS11948SVH12 | 4.0E+05 | 1.5E−03 | 3.7E−09 |
| | AS11948SVH12M8 | 4.3E+05 | 9.5E−03 | 2.2E−08 |
| | AS11948SVH12M9 | 4.0E+05 | 6.3E−03 | 1.6E−08 |

TABLE 19
Affinity ranking of humanized sdAbs (AS06617)

| Ligand | Analyte | kₐ (1/Ms) | k_{d} (1/s) | K_{D} (M) |
|---|---|---|---|---|
| PD-L1/His | AS06617 | 3.10E+05 | 4.30E−04 | 1.40E−09 |
| | AS06617VH11 | 4.30E+05 | 1.20E−03 | 2.70E−09 |

TABLE 20
Affinity ranking of humanized sdAbs (AS06775)

| Ligand | Analyte | kₐ (1/Ms) | k_{d} (1/s) | K_{D} (M) |
|---|---|---|---|---|
| PD-L1/His | AS0775 | 2.10E+05 | 3.60E−04 | 1.70E−09 |
| | AS06775VH11 | 3.20E+05 | 5.00E−04 | 1.60E−09 |
| | AS06775VH4 | 2.46E+05 | 7.16E−04 | 2.92E−09 |

### Affinity Determination

AS06730S, AS06730SVH3a, AS06730SVH12, AS06730AVH12M8, AS06730SVH12M9, AS06750VH2, AS06750VH11, AS06750VH4, AS11948S, AS11948SVH12, AS11948SV12M8, AS11948SV12M9, AS06617VH11, AS06775VH11 and AS06775VH4 were selected for affinity determination. Affinity constant (K_{d}) of each HCAbs was determined by surface plasmon resonance (SPR) on a BIAcore T200 instrument. Briefly, for most of HCAbs affinity determination, PD-L1 His was amine-coupled to a CM5 sensor chip at a density of no higher than 100 RU. Anti-PD-L1 HCAbs were injected at 5 different concentrations between 0.11 nM and 27 nM. Flow rate was 30111/min in all experiments. Association and dissociation phases were 5 and 10 min, respectively. The chip was regenerated using Glycine/HCl pH 1.5. For AS06730SVH12, AS06730SVH12M8, AS06730VH12M9, AS11948SV12, AS11948SV12M8 and AS11948SV12M9 HCAbs affinity determination, anti-PD-L1 HCAbs were captured on a CM5 sensor chip at a density of no higher than 100 RU by anti-human IgG antibody. Anti-PD-L1 His was injected at 5 different concentrations between 0.33 and 27 nM. Flow rate was 30111/min in all experiments. Association and dissociation phases were 5 min. Binding curves at different concentrations of HCAbs were used to calculate the kinetic parameters kₒₙ, k_{off} and K_{d}. The kinetics data were summarized in Table 21 and Table 22.

TABLE 21
Affinity parameters of humanized HCAbs

| Ligand | Analyte | kₐ (1/Ms) | k_{d} (1/s) | K_{D} (M) |
|---|---|---|---|---|
| PD-L1 His | AS06730 HCAb | 7.6E+04 | 1.7E−04 | 2.2E−09 |
| | AS06730S HCAb | 7.1E+04 | 3.5E−04 | 4.9E−09 |
| | AS06730SVH3a HCAb | 6.2E+04 | 3.7E−04 | 5.9E−09 |
| | AS06750 HCAb | 2.0E+05 | 3.6E−04 | 1.8E−09 |
| | AS06750VH2 HCAb | 2.0E+05 | 3.3E−04 | 1.7E−09 |
| | AS06750VH11 HCAb | 9.5E+04 | 3.0E−04 | 3.2E−09 |
| | AS06750VH4 HCAb | 1.4E+05 | 2.7E−04 | 1.9E−09 |
| | AS11948 HCAb | 3.5E+05 | 6.2E−04 | 1.8E−09 |
| | AS11948S HCAb | 2.9E+05 | 1.1E−03 | 3.8E−09 |
| | AS06617 HCAb | 4.8E+05 | 1.0E−03 | 2.1E−09 |
| | AS06617VH11 HCAb | 4.3E+05 | 1.2E−03 | 2.7E−09 |
| | AS06775 HCAb | 3.1E+05 | 4.3E−04 | 1.4E−09 |
| | AS06775VH4 HCAb | 2.1E+05 | 3.6E−04 | 1.7E−09 |
| | AS06775VH11 HCAb | 3.2E+05 | 5.0E−04 | 1.6E−09 |

TABLE 22
Affinity parameters of humanized HCAbs

| | | kₐ | k_{d} | K_{D} |
|---|---|---|---|---|
| Ligand | Analyte | (1/Ms) | (1/s) | (M) |
| AS06730SVH12 | PD-L1 His | 3.0E+05 | 1.8E−03 | 6.1E−09 |
| HCAb | | | | |
| AS06730SVH12M8 | | 3.6E+05 | 6.5E−03 | 1.8E−08 |
| HCAb | | | | |
| AS06730SVH12M9 | | 4.0E+05 | 1.2E−02 | 3.0E−08 |
| HCAb | | | | |
| AS11948SVH12 | | 4.0E+05 | 1.5E−03 | 3.7E−09 |
| HCAb | | | | |
| AS11948SVH12M8 | | 4.3E+05 | 9.5E−03 | 2.2E−08 |
| HCAb | | | | |
| AS11948SVH12M9 | | 4.0E+05 | 6.3E−03 | 1.6E−08 |
| HCAb | | | | |

### PD-L1 Based Blockade Assay

PD-L1 based blockade assay was performed as described in Example 9. All the selected humanized anti-PD-L1 HCAbs are comparable to Tecentriq® in inhibiting the binding between PD-L1 and PD-1. The EC₅₀ data was summarized in Table 23.

TABLE 23
EC₅₀ of PD-L1- based blockade assay for HCAbs

| Sample | AS0730 HCAb | AS06730S HCAb | AS06730SVH3a HCAb | AS06730SVH12 HCAb | AS06750 HCAb |
|---|---|---|---|---|---|
| EC₅₀ (M) | 2.15E−09 | 2.54E−09 | 1.58E−09 | 4.60E−09 | 3.60E−09 |

| Sample | AS06750VH2 HCAb | AS06750VH11 HCAb | AS06750H4 HCAb | AS11948 HCAb | AS11948S HCAb |
|---|---|---|---|---|---|
| EC₅₀ (M) | 4.44E−09 | 3.61E−09 | 5.77E−09 | 5.00E−09 | 3.28E−09 |

| Sample | AS11948VH12 HCAb | AS11948VH12M8 HCAb | AS06617 HCAb | AS06617VH11 HCAb | AS06775 HCAb |
|---|---|---|---|---|---|
| EC₅₀ (M) | 2.91E−09 | 7.86E−09 | 3.86E−09 | 4.28E−09 | 3.08E−09 |

| Sample | AS06775VH11 HCAb | AS06775VH4 HCAb | Tecentriq |
|---|---|---|---|
| EC₅₀ (M) | 5.26E−09 | 3.43E−09 | 5.92E−09 |

### In vivo Activity of Humanized HCAbs

In the studies presented here, the efficacy of PD-L1 HCAb blockade against murine tumor model was investigated. Inhibition of the PD-L1 interaction is proposed to exert a therapeutic effect by restoring anti-tumor CD8+ T cell responses, thus the preclinical efficacy study was conducted in syngeneic murine tumor model in which the immune system of the host is fully intact. The human PD-1 transgenic mice were used.

In this study, mice were inoculated subcutaneously in the right flank with 1×10⁶ human PD-L1 overexpression MC38 colon carcinoma cells. When tumors reached a mean volume of ˜100 mm³, mice were sorted into treatment groups (n=5) (defined as study day 0). 6 humanized HCAbs tested in this study were listed: AS06730QVH1, AS06750VH11, AS11948SVH12, AS06617VH11, AS06617VH11, AS11948QVH1 and AS06775VH11. Groups were administered benchmark antibody MEDI4736 (10 mg/kg) or humanized HCAbs (5.33 mg/kg) intravenously days 0, 2, 5, 7, 9 and 12. A control group was treated with 10 ml/kg of PBS. Tumors were measured twice weekly for the study duration. All treatment groups demonstrated significant efficacy (P<0.050) when compared to the control group. These observations support that anti-PD-L1 therapy as an effective strategy for driving anti-tumor CD8+ T cell responses.

TABLE 24
CDRs of isolated sdAbs

| sdAb | ID | CDR1 | ID | CDR2 | ID | CDR3 |
|---|---|---|---|---|---|---|
| AS06617 | 169 | GRIFISYAVG | 269 | GIRWNGIHTDYADSVKG | 369 | HRTIATIPEKYEYEY |
| AS06618 | 170 | GRTFLSYAVG | 270 | GIRWSGGYTDYAEAVKG | 370 | HRTIATIPEKYEYEY |
| AS06624 | 171 | GRTFLTYALG | 271 | GVSWSGSGTKYADSVKG | 371 | QISAIVPISAHEYEY |
| AS06628 | 172 | GRTFITYAIG | 272 | AINWSGSMTSYADSVKG | 372 | HRGAIAPMTQSVYDY |
| AS06639 | 173 | GRTFITYAIG | 273 | AINWSGSMTSYADSVKG | 373 | HRGAIAPMTQSVYDT |
| AS06682 | 174 | GRTFLSYAVG | 274 | GIRWSGEHTDYAASVKG | 374 | HTTIATIPKKYEYEY |
| AS06686 | 175 | GRTFLTYALG | 275 | GVSWSGSSTKYADSVKG | 375 | QISAIVPISAHEYQY |
| AS06703 | 176 | GRTFITYAIG | 276 | AINWSGSMTSYADSVKG | 376 | HLGAIAPMSQSVYDY |
| AS06709 | 177 | GRTFLSYAVG | 277 | GIRWSGGSTDYSDSVKG | 377 | HRTIATIPEKYEYEY |
| AS06730 | 178 | GRTFITYAIG | 278 | AINWSGSMTSYADSVKG | 378 | HRGAIAPIAQSVYTN |
| AS06750 | 179 | GRTFLTYAVG | 279 | GIRWSGGYTDYADSVKG | 379 | HRTIATIPEKYEYEY |
| AS06752 | 180 | GRTFLTYAVG | 280 | GIRWSGESTDYAESVKG | 380 | HRTIATIPEKYYYEY |
| AS06763 | 181 | GRPVSSAVMG | 281 | RLTSSATSTFYAESVKG | 381 | DVPGTKIWSIQTPDRYNY |
| AS06766 | 182 | GRTLTGLLIG | 282 | IISWTYGSTNYADSVKG | 382 | RDVAVAKYDS |
| AS06775 | 183 | GRTFLTLAVG | 283 | GIRWSGSGTDYADSVKG | 383 | HTTIATIPEKYEYEY |
| AS06778 | 184 | GRTFITYAMG | 284 | AISWSGSSTYSADSVKG | 384 | EVSARTGEHLPKLMGDY |
| AS06786 | 185 | GRTFLTLAVG | 285 | GIRWSGSGTDYADSVKG | 385 | HTTIATIPEKYEYEY |
| AS06791 | 186 | GRTFITYAIG | 286 | AINWSGSMTSYADSVKG | 386 | HRGAIAPMTQSVYDY |
| AS06808 | 187 | GRIFSRYAMG | 287 | TSTGSGGLISYANSVKG | 387 | NRYNSDSRYMSSYDW |
| AS06810 | 188 | GRTFLSYAVG | 288 | GIRWSGLHTDYADSVKG | 388 | HRTIATIPEKYEYEY |
| AS11947 | 189 | GRTFISYAVG | 289 | GIRWNGISTDYTDSVKG | 389 | HRTIATIPNKYEYDH |
| AS11948 | 190 | GRIFVTYGMG | 290 | AINWSGSMTSYGDSVKG | 390 | ALGAVVYTTREPYTY |
| AS12003 | 191 | GRTFLSYAVG | 291 | GIRWSGGSTDYADSVKG | 391 | HRTIATVPNKYEYDT |
| AL22863 | 192 | VSSFSINDMG | 292 | TIAS-GGSTNYADSVKG | 392 | DERDWTRRRYSY |
| AL23474 | 193 | GRTFSNYTMA | 293 | VVSRGGGATDYADSVKG | 393 | GTDLSYYYSTKKWAY |
| AS06730S | 194 | GRTFITYAIG | 294 | AISWSGSMTSYADSVKG | 394 | HRGAIAPIAQSVYTN |
| AS06730Q | 195 | GRTFITYAIG | 295 | AIQWSGSMTSYADSVKG | 395 | HRGAIAPIAQSVYTN |
| AS06730QVH1 | 196 | GRTFITYAIG | 296 | AIQWSGSMTSYADSVKG | 396 | HRGAIAPIAQSVYTN |
| AS06730QVH2 | 197 | GRTFITYAIG | 297 | AIQWSGSMTSYADSVKG | 397 | HRGAIAPIAQSVYTN |
| AS06730QVH3a | 198 | GRTFITYAIG | 298 | AIQWSGSMTSYADSVKG | 398 | HRGAIAPIAQSVYTN |
| AS06730SVH12 | 199 | GRTFITYAIG | 299 | AISWSGSMTSYADSVKG | 399 | HRGAIAPIAQSVYTN |
| AS06730SVH12M8 | 200 | GRTFITYAIG | 300 | AISWSGSITSYADSVKG | 400 | HRGAIAPIAQSVYTN |
| AS06730SVH12M9 | 201 | GRTFITYAIG | 301 | AISWSGSLTSYADSVKG | 401 | HRGAIAPIAQSVYTN |
| AS06750VH1 | 202 | GRTFLTYAVG | 302 | GIRWSGGYTDYADSVKG | 402 | HRTIATIPEKYEYEY |
| AS06750VH2 | 203 | GRTFLTYAVG | 303 | GIRWSGGYTDYADSVKG | 403 | HRTIATIPEKYEYEY |
| AS06750VH3 | 204 | GRTFLTYAVG | 304 | GIRWSGGYTDYADSVKG | 404 | HRTIATIPEKYEYEY |
| AS06750VHa | 205 | GRTFLTYAVG | 305 | GIRWSGGYTDYADSVKG | 405 | HRTIATIPEKYEYEY |
| AS06750VH11 | 206 | GRTFLTYAVG | 306 | GIRWSGGYTDYADSVKG | 406 | HRTIATIPEKYEYEY |
| AS11948A | 207 | GRIFVTYGMG | 307 | AIAWSGSMTSYGDSVKG | 407 | ALGAVVYTTREPYTY |
| AS11948S | 208 | GRTFVTYGMG | 308 | AISWSGSMTSYGDSVKG | 408 | ALGAVVYTTREPYTY |
| AS11948Q | 209 | GRTFVTYGMG | 309 | AIQWSGSMTSYGDSVKG | 409 | ALGAVVYTTREPYTY |
| AS11948QVH1 | 210 | GRTFVTYGMG | 310 | AIQWSGSMTSYGDSVKG | 410 | ALGAVVYTTREPYTY |
| AS11948QVH2 | 211 | GRIFVTYGMG | 311 | AIQWSGSMTSYGDSVKG | 411 | ALGAVVYTTREPYTY |
| AS11948QVHa | 212 | GRIFVTYGMG | 312 | AIQWSGSMTSYGDSVKG | 412 | ALGAVVYTTREPYTY |
| AS11948SVH12 | 213 | GRIFVTYGMG | 313 | AISWSGSMTSYGDSVKG | 413 | ALGAVVYTTREPYTY |
| AS11948SVH12M8 | 214 | GRTFVTYGMG | 314 | AISWSGSITSYGDSVKG | 414 | ALGAVVYTTREPYTY |
| AS11948SVH12M9 | 215 | GRIFVTYGMG | 315 | AISWSGSLTSYGDSVKG | 415 | ALGAVVYTTREPYTY |
| AS06617VH11 | 216 | GRTFISYAVG | 316 | GIRWSGIHTDYADSVKG | 416 | HRTIATIPEKYEYEY |
| AS06775VH11 | 217 | GRTFLTLAVG | 317 | GIRWSGSGTDYADSVKG | 417 | HTTIATIPEKYEYEY |
| AS06775VH4 | 218 | GRTFLTYAVG | 318 | GIRWSGGYTDYADSVKG | 418 | HRTIATIPEKYEYEY |

ID: SEQ ID NO

TABLE 25
Framework Regions 1 and 2

| sdAb | ID | FR-1 | ID | FR-2 |
|---|---|---|---|---|
| AS06617 | 119 | DVQLVESGGGLVQAGDSLRLSCAAS | 219 | WFRQAPGSEREFVA |
| AS06618 | 120 | EVQLVESGGRLVRAGDSLRLSCAAS | 220 | WFRQAPGTEREFVA |
| AS06624 | 121 | QVQLVESGGGLVQAGGSLRLACSAS | 221 | WFRQAPGKEREFVA |
| AS06628 | 122 | QVQLVESGGGLVQAGGSLRLSCAAS | 222 | WFRQAPGKEREFVT |
| AS06639 | 123 | AVQLVESGGGLVQAGGSLRLSCAAS | 223 | WFRQAPGKEREFVS |
| AS06682 | 124 | AVQLVESGGGLVQAGDSLRLSCTAS | 224 | WFRQAPGTEREFVA |
| AS06686 | 125 | AVQLVESGGGLVQAGDSLRLACAAS | 225 | WFRQAPGKEREFVA |
| AS06703 | 126 | EVQLVESGGGLVRAGGSLRLSCAAS | 226 | WFRQAPGKEREFVT |
| AS06709 | 127 | EVQLVESGGGLVQAGDSLRLSCTAS | 227 | WFRQAPGTEREFVA |
| AS06730 | 128 | QVQLVESGGGLVQAGGSLRLSCAAS | 228 | WFRQAPGKEREFVS |
| AS06750 | 129 | AVQLVESGGGLVQAGDSLRLSCTAS | 229 | WFRQAPGTEREFVA |
| AS06752 | 130 | EVQLVESGGGLVQAGDSLRLSCAAS | 230 | WFRQAPGTEREFVA |
| AS06763 | 131 | QVQLVESGGGLVQAGGSLRLSCAVS | 231 | WFRQAPGKEREFVG |
| AS06766 | 132 | EVQLVESGGGLVQAGGSLSLSCAVS | 232 | WFRQAPGKERELVA |
| AS06775 | 133 | QVQLVESGGGLVQAGDSLRLSCAAS | 233 | WFRQAPGTEREFVA |
| AS06778 | 134 | QVQLVESGGGLVQAGGSLKLSCAAS | 234 | WFRQAPGKERELVA |
| AS06786 | 135 | QVQLVESGGGLVQAGDSLRLSCAAS | 235 | WFRQAPGTEREFVA |
| AS06791 | 136 | QVQLVESGGGLVQAGGSLRLSCAAS | 236 | WFRQAPGKEREFVT |
| AS06808 | 137 | QVKLEESGGGLVQAGGSLRLSCVAS | 237 | WFRQAPGKEREFVS |
| AS06810 | 138 | AVQLVESGGGLVQAGDSLRLSCAAS | 238 | WFRQAPGTEREFVA |
| AS11947 | 139 | DVQLVESGGGLVQAGDSLRLTCSAS | 239 | WFRQAPGTEREFVA |
| AS11948 | 140 | EVQLVESGGGLVQAGDSLRLSCVAS | 240 | WFRQAPGKEREFVA |
| AS12003 | 141 | EVQLVESGGGLVQAGDSLRLSCAAS | 241 | WFRQAPGTEREFVA |
| AL22863 | 142 | QVKLEESGGGLVQVGDSLRLSCAAS | 242 | WYRQAPGKQRELVA |
| AL23474 | 143 | QVKLEESGGGLVQVGDSLRLSCAAS | 243 | WFRQFPGKEREFVA |
| AS06730S | 144 | QVQLVESGGGLVQAGGSLRLSCAAS | 244 | WFRQAPGKEREFVS |
| AS06730Q | 145 | QVQLVESGGGLVQAGGSLRLSCAAS | 245 | WFRQAPGKEREFVS |
| AS06730QVH1 | 146 | EVQLVESGGGLVQPGGSLRLSCAAS | 246 | WVRQAPGKGLEWVS |
| AS06730QVH2 | 147 | EVQLVESGGGLVQPGGSLRLSCAAS | 247 | WFRQAPGKGLEWVS |
| AS06730QVH3a | 148 | EVQLVESGGGLVQPGGSLRLSCAAS | 248 | WFRQAPGKGLEFVS |
| AS06730SVH12 | 149 | EVQLVESGGGLVQPGGSLRLSCAAS | 249 | WFRQAPGKGREFVS |
| AS06730SVH12M8 | 150 | EVQLVESGGGLVQPGGSLRLSCAAS | 250 | WFRQAPGKGREFVS |
| AS06730SVH12M9 | 151 | EVQLVESGGGLVQPGGSLRLSCAAS | 251 | WFRQAPGKGREFVS |
| AS06750VH1 | 152 | EVQLVESGGGLVQPGGSLRLSCAAS | 252 | WVRQAPGKGLEWVS |
| AS06750VH2 | 153 | EVQLVESGGGLVQPGGSLRLSCAAS | 253 | WFRQAPGKGLEWVA |
| AS06750VH3 | 154 | EVQLVESGGGLVQPGGSLRLSCAAS | 254 | WFRQAPGKGLEFVA |
| AS06750VHa | 155 | EVQLVESGGGLVQPGGSLRLSCTAS | 255 | WFRQAPGKGLEFVA |
| AS06750VH11 | 156 | EVQLVESGGGLVQPGGSLRLSCAAS | 256 | WFRQAPGKGREFVS |
| AS11948A | 157 | EVQLVESGGGLVQAGDSLRLSCVAS | 257 | WFRQAPGKEREFVA |
| AS11948S | 158 | EVQLVESGGGLVQAGDSLRLSCVAS | 258 | WFRQAPGKEREFVA |
| AS11948Q | 159 | EVQLVESGGGLVQAGDSLRLSCVAS | 259 | WFRQAPGKEREFVA |
| AS11948QVH1 | 160 | EVQLVESGGGLVQPGGSLRLSCAAS | 260 | WVRQAPGKGLEWVS |
| AS11948QVH2 | 161 | EVQLVESGGGLVQPGGSLRLSCAAS | 261 | WFRQAPGKGLEFVA |
| AS11948QVHa | 162 | EVQLVESGGGLVQPGGSLRLSCVAS | 262 | WFRQAPGKGREFVS |
| AS11948SVH12 | 163 | EVQLVESGGGLVQPGGSLRLSCAAS | 263 | WFRQAPGKGREFVS |
| AS11948SVH12M8 | 164 | EVQLVESGGGLVQPGGSLRLSCAAS | 264 | WFRQAPGKGREFVS |
| AS11948SVH12M9 | 165 | EVQLVESGGGLVQPGGSLRLSCAAS | 265 | WFRQAPGKGREFVS |
| AS06617VH11 | 166 | EVQLVESGGGLVQPGGSLRLSCAAS | 266 | WFRQAPGKGREFVS |
| AS06775VH11 | 167 | EVQLVESGGGLVQPGGSLRLSCAAS | 267 | WFRQAPGKGREFVS |
| AS06775VH4 | 168 | EVQLVESGGGLVQPGGSLRLSCAAS | 268 | WFRQAPGKGLEFVS |

ID: SEQ ID NO; FR: Framework region

TABLE 26
Framework Regions 3 and 4

| sdAb | ID | FR-3 | ID | FR-4 |
|---|---|---|---|---|
| AS06617 | 319 | RFTISRDNAKNTVTLEMTSLKPEDTAVYYCAA | 419 | WGQGTQVTVSS |
| AS06618 | 320 | RFTISRDNAKNTVYLQMNSLKPEDTAVYYCAA | 420 | WGQGTQVTVSS |
| AS06624 | 321 | RFTISRDNAKNTVYMQMNSLKPEDTAVYYCAA | 421 | WGQGTQVTVSS |
| AS06628 | 322 | RFTISRDNNKNMVYLQMNSLKPEDTAVYYCAA | 422 | WGQGTQVTVSS |
| AS06639 | 323 | RFTISRDNAKNTVYLQMNGLKPEDTAVYYCAA | 423 | WGQGTQVTVSS |
| AS06682 | 324 | RFTISRDNAKNTVYLQMNSLKPEDTAVYYCAA | 424 | WGQGTQVTVSS |
| AS06686 | 325 | RFTISRDNAKNTVYMQMNSLKPEDTAVYYCAA | 425 | WGQGTQVTVSS |
| AS06703 | 326 | RFTISRDNNKNTVYLQMNSLKPEDTALYYCAA | 426 | WGQGTQVTVSS |
| AS06709 | 327 | RFTISRDNAKNTVYLQMNSLKPEDTAVYYCAA | 427 | WGQGTQVTVSS |
| AS06730 | 328 | RFTISRDNAKNTVYLQMNGLKPEDTAVYYCAA | 428 | WGQGTQVTVSS |
| AS06750 | 329 | RFTISRDNAKNTVYLQMNSLKPEDTAVYYCAA | 429 | WGQGTQVTVSS |
| AS06752 | 330 | RFTISRDDTKNTVYLQMNSLKPEDTAVYYCAA | 430 | WGQGTQVTVSS |
| AS06763 | 331 | RFTISRDNAKNTVYLQMNNLKPEDTAVYYCAA | 431 | WGQGTQVTVSS |
| AS06766 | 332 | RFTISRDNAKNTVLLQMNSLKPEDTAVYYCSA | 432 | WGQGTQVTVSS |
| AS06775 | 333 | RFTISRDNAKNTVYLQMNSLKPEDTAVYYCAA | 433 | WGRGTQVTVSS |
| AS06778 | 334 | RFTISRDNAKNTVYLQMNSLKPEDTAVYYCAA | 434 | WGQGTQVTVSS |
| AS06786 | 335 | RFTISRDNAANTVYLQMNSLKPEDTAVYYCAA | 435 | WGQGTQVTVSS |
| AS06791 | 336 | RFTISRDNAKNTVYLQINGLKSEDTAVYYCAA | 436 | WGQGTQVTVSS |
| AS06808 | 337 | RFTISRDNAKNTVYLQMNNLKPEDTAIYYCAA | 437 | WGQGTQVTVSS |
| AS06810 | 338 | RFTISRDNAKNTVYLQMNSLKPEDTAIYYCAA | 438 | WGQGTQVTVSS |
| AS11947 | 339 | RFTISRDNAKNTVYLQMNSLKPEDTAIYYCAA | 439 | WGQGTQVTVSS |
| AS11948 | 340 | RFAISRDNAKNTVYLQMNSLKPEDTAVYYCAA | 440 | WGRGTQVTVSS |
| AS12003 | 341 | RFTISRDNAKNTVYLQMNSLKPEDTAVYYCAA | 441 | WGQGTQVTVSS |
| AL22863 | 342 | RFTISRDNVKNTVYLQMNGLKPEDTAVYYCNA | 442 | WGQGTQVTVSS |
| AL23474 | 343 | RFTISRDNAKNTMYLQMNSLKTEDTAVYYCAA | 443 | WGQGTQVTVSS |
| AS06730S | 344 | RFTISRDNAKNTVYLQMNGLKPEDTAVYYCAA | 444 | WGQGTQVTVSS |
| AS06730Q | 345 | RFTISRDNAKNTVYLQMNGLKPEDTAVYYCAA | 445 | WGQGTQVTVSS |
| AS06730QVH1 | 346 | RFTISRDNSKNTLYLQMNSLRAEDTAVYYCAK | 446 | WGQGTLVTVSS |
| AS06730QVH2 | 347 | RFTISRDNSKNTVYLQMNSLRAEDTAVYYCAA | 447 | WGQGTLVTVSS |
| AS06730QVH3a | 348 | RFTISRDNSKNTVYLQMNSLRAEDTAVYYCAA | 448 | WGQGTLVTVSS |
| AS06730SVH12 | 349 | RFTISRDNAKNTLYLQMNSLRPEDTAVYYCAA | 449 | WGQGTLVTVSS |
| AS06730SVH12M8 | 350 | RFTISRDNAKNTLYLQMNSLRPEDTAVYYCAA | 450 | WGQGTLVTVSS |
| AS06730SVH12M9 | 351 | RFTISRDNAKNTLYLQMNSLRPEDTAVYYCAA | 451 | WGQGTLVTVSS |
| AS06750VH1 | 352 | RFTISRDNSKNTLYLQMNSLRAEDTAVYYCAK | 452 | WGQGTLVTVSS |
| AS06750VH2 | 353 | RFTISRDNSKNTVYLQMNSLRAEDTAVYYCAA | 453 | WGQGTLVTVSS |
| AS06750VH3 | 354 | RFTISRDNSKNTVYLQMNSLRAEDTAVYYCAA | 454 | WGQGTLVTVSS |
| AS06750VHa | 355 | RFTISRDNSKNTVYLQMNSLRAEDTAVYYCAA | 455 | WGQGTLVTVSS |
| AS06750VH11 | 356 | RFTISRDNAKNTLYLQMNSLRPEDTAVYYCAA | 456 | WGQGTLVTVSS |
| AS11948A | 357 | RFAISRDNAKNTVYLQMNSLKPEDTAVYYCAA | 457 | WGRGTQVTVSS |
| AS11948S | 358 | REAISRDNAKNTVYLQMNSLKPEDTAVYYCAA | 458 | WGRGTQVTVSS |
| AS11948Q | 359 | RFAISRDNAKNTVYLQMNSLKPEDTAVYYCAA | 459 | WGRGTQVTVSS |
| AS11948QVH1 | 360 | RFTISRDNSKNTLYLQMNSLRAEDTAVYYCAK | 460 | WGQGTLVTVSS |
| AS11948QVH2 | 361 | RFTISRDNSKNTVYLQMNSLRAEDTAVYYCAA | 461 | WGQGTLVTVSS |
| AS11948QVHa | 362 | RFTISRDNSKNTVYLQMNSLRAEDTAVYYCAA | 462 | WGQGTLVTVSS |
| AS11948SVH12 | 363 | RFTISRDNAKNTLYLQMNSLRPEDTAVYYCAA | 463 | WGQGTLVTVSS |
| AS11948SVH12M8 | 364 | RFTISRDNAKNTLYLQMNSLRPEDTAVYYCAA | 464 | WGQGTLVTVSS |
| AS11948SVH12M9 | 365 | RFTISRDNAKNTLYLQMNSLRPEDTAVYYCAA | 465 | WGQGTLVTVSS |
| AS06617VH11 | 366 | RFTISRDNSKNTLYLQMNSLRAEDTAVYYCAA | 466 | WGQGTLVTVSS |
| AS06775VH11 | 367 | RFTISRDNAKNTLYLQMNSLRPEDTAVYYCAA | 467 | WGQGTLVTVSS |
| AS06775VH4 | 368 | RFTISRDNSKNTLYLQMNSLRAEDTAVYYCAA | 468 | WGQGTLVTVSS |

ID: SEQ ID NO; FR: Framework region

TABLE 27
sdAbs

| sdAb | ID | Sequence |
|---|---|---|
| AS06617 | 469 | DVQLVESGGGLVQAGDSLRLSCAASGRIFISYAVGWERQAPGSEREFVAGIRWNGIH |
| | | TDYADSVKGRFTISRDNAKNTVTLEMTSLKPEDTAVYYCAAHRTIATIPEKYEYEYW |
| | | GQGTQVTVSS |
| AS06618 | 470 | EVQLVESGGRLVRAGDSLRLSCAASGRTFLSYAVGWFRQAPGTEREFVAGIRWSGGY |
| | | TDYAEAVKGRFTISRDNAKNTVYLQMNSLKPEDTAVYYCAAHRTIATIPEKYEYEYW |
| | | GQGTQVTVSS |
| AS06624 | 471 | QVQLVESGGGLVQAGGSLRLACSASGRTELTYALGWERQAPGKEREFVAGVSWSGSG |
| | | TKYADSVKGRFTISRDNAKNTVYMQMNSLKPEDTAVYYCAAQISAIVPISAHEYEYW |
| | | GQGTQVTVSS |
| AS06628 | 472 | QVQLVESGGGLVQAGGSLRLSCAASGRTFITYAIGWFRQAPGKEREFVTAINWSGSM |
| | | TSYADSVKGRETISRDNNKNMVYLQMNSLKPEDTAVYYCAAHRGAIAPMTQSVYDYW |
| | | GQGTQVTVSS |
| AS06639 | 473 | AVQLVESGGGLVQAGGSLRLSCAASGRTFITYAIGWFRQAPGKEREFVSAINWSGSM |
| | | TSYADSVKGRFTISRDNAKNTVYLQMNGLKPEDTAVYYCAAHRGAIAPMTQSVYDTW |
| | | GQGTQVTVSS |
| AS06682 | 474 | AVQLVESGGGLVQAGDSLRLSCTASGRTFLSYAVGWFRQAPGTEREFVAGIRWSGEH |
| | | TDYAASVKGRETISRDNAKNTVYLQMNSLKPEDTAVYYCAAHTTIATIPKKYEYEYW |
| | | GQGTQVTVSS |
| AS06686 | 475 | AVQLVESGGGLVQAGDSLRLACAASGRTELTYALGWFRQAPGKEREFVAGVSWSGSS |
| | | TKYADSVKGRFTISRDNAKNTVYMQMNSLKPEDTAVYYCAAQISAIVPISAHEYQYW |
| | | GQGTQVTVSS |
| AS06703 | 476 | EVQLVESGGGLVRAGGSLRLSCAASGRTFITYAIGWFRQAPGKEREFVTAINWSGSM |
| | | TSYADSVKGRFTISRDNNKNTVYLQMNSLKPEDTALYYCAAHLGAIAPMSQSVYDYW |
| | | GQGTQVTVSS |
| AS06709 | 477 | EVQLVESGGGLVQAGDSLRLSCTASGRTELSYAVGWFRQAPGTEREFVAGIRWSGGS |
| | | TDYSDSVKGRFTISRDNAKNTVYLQMNSLKPEDTAVYYCAAHRTIATIPEKYEYEYW |
| | | GQGTQVTVSS |
| AS06730 | 478 | QVQLVESGGGLVQAGGSLRLSCAASGRTFITYAIGWERQAPGKEREFVSAINWSGSM |
| | | TSYADSVKGRFTISRDNAKNTVYLQMNGLKPEDTAVYYCAAHRGAIAPIAQSVYTNW |
| | | GQGTQVTVSS |
| AS06750 | 479 | AVQLVESGGGLVQAGDSLRLSCTASGRTFLTYAVGWERQAPGTEREFVAGIRWSGGY |
| | | TDYADSVKGRFTISRDNAKNTVYLQMNSLKPEDTAVYYCAAHRTIATIPEKYEYEYW |
| | | GQGTQVTVSS |
| AS06752 | 480 | EVQLVESGGGLVQAGDSLRLSCAASGRTFLTYAVGWERQAPGTEREFVAGIRWSGES |
| | | TDYAESVKGRFTISRDDTKNTVYLQMNSLKPEDTAVYYCAAHRTIATIPEKYYYEYW |
| | | GQGTQVTVSS |
| AS06763 | 481 | QVQLVESGGGLVQAGGSLRLSCAVSGRPVSSAVMGWFRQAPGKEREFVGRLTSSATS |
| | | TFYAESVKGRFTISRDNAKNTVYLQMNNLKPEDTAVYYCAADVPGTKIWSIQTPDRY |
| | | NYWGQGTQVTVSS |
| AS06766 | 482 | EVQLVESGGGLVQAGGSLSLSCAVSGRTLTGLLIGWFRQAPGKERELVAIISWTYGS |
| | | TNYADSVKGRFTISRDNAKNTVLLQMNSLKPEDTAVYYCSARDVAVAKYDSWGQGTQ |
| | | VTVSS |
| AS06775 | 483 | QVQLVESGGGLVQAGDSLRLSCAASGRTELTLAVGWFRQAPGTEREFVAGIRWSGSG |
| | | TDYADSVKGRETISRDNAKNTVYLQMNSLKPEDTAVYYCAAHTTIATIPEKYEYEYW |
| | | GRGTQVTVSS |
| AS06778 | 484 | QVQLVESGGGLVQAGGSLKLSCAASGRTFITYAMGWFRQAPGKERELVAAISWSGSS |
| | | TYSADSVKGRETISRDNAKNTVYLQMNSLKPEDTAVYYCAAEVSARTGEHLPKLMGD |
| | | YWGQGTQVTVSS |
| AS06786 | 485 | QVQLVESGGGLVQAGDSLRLSCAASGRTELTLAVGWERQAPGTEREFVAGIRWSGSG |
| | | TDYADSVKGRFTISRDNAANTVYLQMNSLKPEDTAVYYCAAHTTIATIPEKYEYEYW |
| | | GQGTQVTVSS |
| AS06791 | 486 | QVQLVESGGGLVQAGGSLRLSCAASGRTFITYAIGWFRQAPGKEREFVTAINWSGSM |
| | | TSYADSVKGRETISRDNAKNTVYLQINGLKSEDTAVYYCAAHRGAIAPMTQSVYDYW |
| | | GQGTQVTVSS |
| AS06808 | 487 | QVKLEESGGGLVQAGGSLRLSCVASGRTFSRYAMGWFRQAPGKEREFVSTSTGSGGL |
| | | TSYANSVKGRFTISRDNAKNTVYLQMNNLKPEDTAIYYCAANRYNSDSRYMSSYDWW |
| | | GQGTQVTVSS |
| AS06810 | 488 | AVQLVESGGGLVQAGDSLRLSCAASGRTFLSYAVGWERQAPGTEREFVAGIRWSGLH |
| | | TDYADSVKGRFTISRDNAKNTVYLQMNSLKPEDTAIYYCAAHRTIATIPEKYEYEYW |
| | | GQGTQVTVSS |
| AS11947 | 489 | DVQLVESGGGLVQAGDSLRLTCSASGRIFISYAVGWERQAPGTEREFVAGIRWNGIS |
| | | TDYTDSVKGRFTISRDNAKNTVYLQMNSLKPEDTAIYYCAAHRTIATIPNKYEYDHW |
| | | GQGTQVTVSS |
| AS11948 | 490 | EVQLVESGGGLVQAGDSLRLSCVASGRIFVTYGMGWFRQAPGKEREFVAAINWSGSM |
| | | TSYGDSVKGREAISRDNAKNTVYLQMNSLKPEDTAVYYCAAALGAVVYTTREPYTYW |
| | | GRGTQVTVSS |
| AS12003 | 491 | EVQLVESGGGLVQAGDSLRLSCAASGRTFLSYAVGWFRQAPGTEREFVAGIRWSGGS |
| | | TDYADSVKGRETISRDNAKNTVYLQMNSLKPEDTAVYYCAAHRTIATVPNKYEYDTW |
| | | GQGTQVTVSS |
| AL22863 | 492 | QVKLEESGGGLVQVGDSLRLSCAASVSSFSINDMGWYRQAPGKQRELVATIASGGST |
| | | NYADSVKGRFTISRDNVKNTVYLQMNGLKPEDTAVYYCNADERDWTRRRYSYWGQGT |
| | | QVTVSS |
| AL23474 | 493 | QVKLEESGGGLVQVGDSLRLSCAASGRTFSNYTMAWERQFPGKEREFVAVVSRGGGA |
| | | TDYADSVKGRFTISRDNAKNTMYLQMNSLKTEDTAVYYCAAGTDLSYYYSTKKWAYW |
| | | GQGTQVTVSS |
| AS06730S | 494 | QVQLVESGGGLVQAGGSLRLSCAASGRTFITYAIGWERQAPGKEREFVSAISWSGSM |
| | | TSYADSVKGRFTISRDNAKNTVYLQMNGLKPEDTAVYYCAAHRGAIAPIAQSVYTNW |
| | | GQGTQVTVSS |
| AS06730Q | 495 | QVQLVESGGGLVQAGGSLRLSCAASGRTFITYAIGWERQAPGKEREFVSAIQWSGSM |
| | | TSYADSVKGRFTISRDNAKNTVYLQMNGLKPEDTAVYYCAAHRGAIAPIAQSVYTNW |
| | | GQGTQVTVSS |
| AS06730QVH1 | 496 | EVQLVESGGGLVQPGGSLRLSCAASGRTFITYAIGWVRQAPGKGLEWVSAIQWSGSM |
| | | TSYADSVKGRFTISRDNSKNTLYLQMNSLRAEDTAVYYCAKHRGAIAPIAQSVYTNW |
| | | GQGTLVTVSS |
| AS06730QVH2 | 497 | EVQLVESGGGLVQPGGSLRLSCAASGRTFITYAIGWFRQAPGKGLEWVSAIQWSGSM |
| | | TSYADSVKGRFTISRDNSKNTVYLQMNSLRAEDTAVYYCAAHRGAIAPIAQSVYTNW |
| | | GQGTLVTVSS |
| AS06730QVH3a | 498 | EVQLVESGGGLVQPGGSLRLSCAASGRTFITYAIGWFRQAPGKGLEFVSAIQWSGSM |
| | | TSYADSVKGRFTISRDNSKNTVYLQMNSLRAEDTAVYYCAAHRGAIAPIAQSVYTNW |
| | | GQGTLVTVSS |
| AS06730SVH12 | 499 | EVQLVESGGGLVQPGGSLRLSCAASGRTFITYAIGWFRQAPGKGREFVSAISWSGSM |
| | | TSYADSVKGRETISRDNAKNTLYLQMNSLRPEDTAVYYCAAHRGAIAPIAQSVYTNW |
| | | GQGTLVTVSS |
| AS06730SVH12M8 | 500 | EVQLVESGGGLVQPGGSLRLSCAASGRTFITYAIGWFRQAPGKGREFVSAISWSGSI |
| | | TSYADSVKGRFTISRDNAKNTLYLQMNSLRPEDTAVYYCAAHRGAIAPIAQSVYTNW |
| | | GQGTLVTVSS |
| AS06730SVH12M9 | 501 | EVQLVESGGGLVQPGGSLRLSCAASGRTFITYAIGWFRQAPGKGREFVSAISWSGSL |
| | | TSYADSVKGRETISRDNAKNTLYLQMNSLRPEDTAVYYCAAHRGAIAPIAQSVYTNW |
| | | GQGTLVTVSS |
| AS06750VH1 | 502 | EVQLVESGGGLVQPGGSLRLSCAASGRTFLTYAVGWVRQAPGKGLEWVSGIRWSGGY |
| | | TDYADSVKGRFTISRDNSKNTLYLQMNSLRAEDTAVYYCAKHRTIATIPEKYEYEYW |
| | | GQGTLVTVSS |
| AS06750VH2 | 503 | EVQLVESGGGLVQPGGSLRLSCAASGRTFLTYAVGWFRQAPGKGLEWVAGIRWSGGY |
| | | TDYADSVKGRFTISRDNSKNTVYLQMNSLRAEDTAVYYCAAHRTIATIPEKYEYEYW |
| | | GQGTLVTVSS |
| AS06750VH3 | 504 | EVQLVESGGGLVQPGGSLRLSCAASGRTFLTYAVGWERQAPGKGLEFVAGIRWSGGY |
| | | TDYADSVKGRFTISRDNSKNTVYLQMNSLRAEDTAVYYCAAHRTIATIPEKYEYEYW |
| | | GQGTLVTVSS |
| AS06750VHa | 505 | EVQLVESGGGLVQPGGSLRLSCTASGRTFLTYAVGWERQAPGKGLEFVAGIRWSGGY |
| | | TDYADSVKGRFTISRDNSKNTVYLQMNSLRAEDTAVYYCAAHRTIATIPEKYEYEYW |
| | | GQGTLVTVSS |
| AS06750VH11 | 506 | EVQLVESGGGLVQPGGSLRLSCAASGRTFLTYAVGWFRQAPGKGREFVSGIRWSGGY |
| | | TDYADSVKGRETISRDNAKNTLYLQMNSLRPEDTAVYYCAAHRTIATIPEKYEYEYW |
| | | GQGTLVTVSS |
| AS11948A | 507 | EVQLVESGGGLVQAGDSLRLSCVASGRIFVTYGMGWERQAPGKEREFVAAIAWSGSM |
| | | TSYGDSVKGRFAISRDNAKNTVYLQMNSLKPEDTAVYYCAAALGAVVYTTREPYTYW |
| | | GRGTQVTVSS |
| AS11948S | 508 | EVQLVESGGGLVQAGDSLRLSCVASGRTFVTYGMGWERQAPGKEREFVAAISWSGSM |
| | | TSYGDSVKGRFAISRDNAKNTVYLQMNSLKPEDTAVYYCAAALGAVVYTTREPYTYW |
| | | GRGTQVTVSS |
| AS11948Q | 509 | EVQLVESGGGLVQAGDSLRLSCVASGRTFVTYGMGWERQAPGKEREFVAAIQWSGSM |
| | | TSYGDSVKGRFAISRDNAKNTVYLQMNSLKPEDTAVYYCAAALGAVVYTTREPYTYW |
| | | GRGTQVTVSS |
| AS11948QVH1 | 510 | EVQLVESGGGLVQPGGSLRLSCAASGRTFVTYGMGWVRQAPGKGLEWVSAIQWSGSM |
| | | TSYGDSVKGRFTISRDNSKNTLYLQMNSLRAEDTAVYYCAKALGAVVYTTREPYTYW |
| | | GQGTLVTVSS |
| AS11948QVH2 | 511 | EVQLVESGGGLVQPGGSLRLSCAASGRTFVTYGMGWFRQAPGKGLEFVAAIQWSGSM |
| | | TSYGDSVKGRETISRDNSKNTVYLQMNSLRAEDTAVYYCAAALGAVVYTTREPYTYW |
| | | GQGTLVTVSS |
| AS11948QVHa | 512 | EVQLVESGGGLVQPGGSLRLSCVASGRTFVTYGMGWERQAPGKGREFVSAIQWSGSM |
| | | TSYGDSVKGRFTISRDNSKNTVYLQMNSLRAEDTAVYYCAAALGAVVYTTREPYTYW |
| | | GQGTLVTVSS |
| AS11948SVH12 | 513 | EVQLVESGGGLVQPGGSLRLSCAASGRTFVTYGMGWFRQAPGKGREFVSAISWSGSM |
| | | TSYGDSVKGRETISRDNAKNTLYLQMNSLRPEDTAVYYCAAALGAVVYTTREPYTYW |
| | | GQGTLVTVSS |
| AS11948SVH12M8 | 514 | EVQLVESGGGLVQPGGSLRLSCAASGRTFVTYGMGWERQAPGKGREFVSAISWSGSI |
| | | TSYGDSVKGRETISRDNAKNTLYLQMNSLRPEDTAVYYCAAALGAVVYTTREPYTYW |
| | | GQGTLVTVSS |
| AS11948SVH12M9 | 515 | EVQLVESGGGLVQPGGSLRLSCAASGRTFVTYGMGWFRQAPGKGREFVSAISWSGSL |
| | | TSYGDSVKGRETISRDNAKNTLYLQMNSLRPEDTAVYYCAAALGAVVYTTREPYTYW |
| | | GQGTLVTVSS |
| AS06617VH11 | 516 | EVQLVESGGGLVQPGGSLRLSCAASGRIFISYAVGWFRQAPGKGREFVSGIRWSGIH |
| | | TDYADSVKGRETISRDNAKNTLYLQMNSLRPEDTAVYYCAAHRTIATIPEKYEYEYW |
| | | GQGTLVTVSS |
| AS06775VH11 | 517 | EVQLVESGGGLVQPGGSLRLSCAASGRTFLTLAVGWERQAPGKGREFVSGIRWSGSG |
| | | TDYADSVKGRETISRDNAKNTLYLQMNSLRPEDTAVYYCAAHTTIATIPEKYEYEYW |
| | | GQGTLVTVSS |
| AS06775VH4 | 518 | EVQLVESGGGLVQPGGSLRLSCAASGRTFLTYAVGWFRQAPGKGLEFVSGIRWSGGY |
| | | TDYADSVKGRFTISRDNSKNTLYLQMNSLRAEDTAVYYCAAHRTIATIPEKYEYEYW |
| | | GQGTLVTVSS |

ID: SEQ ID NO

TABLE 28
HCAbs

| HCAb | ID | Sequence |
|---|---|---|
| AS06617 | 519 | DVQLVESGGGLVQAGDSLRLSCAASGRIFISYAVGWERQAPGSEREFVAGIRWNG |
| | | IHTDYADSVKGRFTISRDNAKNTVTLEMTSLKPEDTAVYYCAAHRTIATIPEKYE |
| | | YEYWGQGTQVTVSSEPKSCDKTHTCPPCPAPELLGGPSVELFPPKPKDTLMISRT |
| | | PEVTCVVVDVSHEDPEVKENWYVDGVEVHNAKTKPREEQYNSTYRVVSVLIVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VESCSVMHEALHNHYTQKSLSLSPGK |
| AS06617VH11 | 520 | EVQLVESGGGLVQPGGSLRLSCAASGRIFISYAVGWFRQAPGKGREFVSGIRWSG |
| | | IHTDYADSVKGRETISRDNAKNTLYLQMNSLRPEDTAVYYCAAHRTIATIPEKYE |
| | | YEYWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLIVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06618 | 521 | EVQLVESGGRLVRAGDSLRLSCAASGRTFLSYAVGWERQAPGTEREFVAGIRWSG |
| | | GYTDYAEAVKGRETISRDNAKNTVYLQMNSLKPEDTAVYYCAAHRTIATIPEKYE |
| | | YEYWGQGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLIVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKITPPVLDSDGSFFLYSKLTVDKSRWQQGN |
| | | VFSCSVMHEALHNHYTQKSLSLSPGK |
| AS06628 | 522 | QVQLVESGGGLVQAGGSLRLSCAASGRIFITYAIGWERQAPGKEREFVTAINWSG |
| | | SMTSYADSVKGRFTISRDNNKNMVYLQMNSLKPEDTAVYYCAAHRGAIAPMTQSV |
| | | YDYWGQGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VESCSVMHEALHNHYTQKSLSLSPGK |
| AS06682 | 523 | AVQLVESGGGLVQAGDSLRLSCTASGRIELSYAVGWFRQAPGTEREFVAGIRWSG |
| | | EHTDYAASVKGRFTISRDNAKNTVYLQMNSLKPEDTAVYYCAAHTTIATIPKKYE |
| | | YEYWGQGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLIVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06686 | 524 | AVQLVESGGGLVQAGDSLRLACAASGRTFLTYALGWFRQAPGKEREFVAGVSWSG |
| | | SSTKYADSVKGRFTISRDNAKNTVYMQMNSLKPEDTAVYYCAAQISAIVPISAHE |
| | | YQYWGQGTQVTVSSEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06703 | 525 | EVQLVESGGGLVRAGGSLRLSCAASGRTFITYAIGWFRQAPGKEREFVTAINWSG |
| | | SMTSYADSVKGRFTISRDNNKNTVYLQMNSLKPEDTALYYCAAHLGAIAPMSQSV |
| | | YDYWGQGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLIVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06730 | 526 | QVQLVESGGGLVQAGGSLRLSCAASGRIFITYAIGWFRQAPGKEREFVSAINWSG |
| | | SMTSYADSVKGRFTISRDNAKNTVYLQMNGLKPEDTAVYYCAAHRGAIAPIAQSV |
| | | YTNWGQGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06750 | 527 | AVQLVESGGGLVQAGDSLRLSCTASGRIFLTYAVGWFRQAPGTEREFVAGIRWSG |
| | | GYTDYADSVKGRFTISRDNAKNTVYLQMNSLKPEDTAVYYCAAHRTIATIPEKYE |
| | | YEYWGQGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLIVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06775 | 528 | QVQLVESGGGLVQAGDSLRLSCAASGRTELTLAVGWERQAPGTEREFVAGIRWSG |
| | | SGTDYADSVKGRETISRDNAKNTVYLQMNSLKPEDTAVYYCAAHTTIATIPEKYE |
| | | YEYWGRGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06775VH4 | 529 | EVQLVESGGGLVQPGGSLRLSCAASGRIFLTLAVGWFRQAPGKGREFVSGIRWSG |
| | | SGTDYADSVKGRFTISRDNAKNTLYLQMNSLRPEDTAVYYCAAHTTIATIPEKYE |
| | | YEYWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06775VH11 | 530 | EVQLVESGGGLVQPGGSLRLSCAASGRTFLTYAVGWFRQAPGKGLEFVSGIRWSG |
| | | GYTDYADSVKGRFTISRDNSKNTLYLQMNSLRAEDTAVYYCAAHRTIATIPEKYE |
| | | YEYWGQGTLVTVSSEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06778 | 531 | QVQLVESGGGLVQAGGSLKLSCAASGRTFITYAMGWFRQAPGKERELVAAISWSG |
| | | SSTYSADSVKGRFTISRDNAKNTVYLQMNSLKPEDTAVYYCAAEVSARTGEHLPK |
| | | LMGDYWGQGTQVTVSSEPKSCDKTHTCPPCPAPELLGGPSVFLFPPKPKDTLMIS |
| | | RTPEVTCVVVDVSHEDPEVKENWYVDGVEVHNAKTKPREEQYNSTYRVVSVLTVL |
| | | HQDWLNGKEYKCKVSNKALPAPIEKTISKAKGQPREPQVYTLPPSRDELTKNQVS |
| | | LTCLVKGFYPSDIAVEWESNGQPENNYKTTPPVLDSDGSFFLYSKLTVDKSRWQQ |
| | | GNVFSCSVMHEALHNHYTQKSLSLSPGK |
| AS06791 | 532 | QVQLVESGGGLVQAGGSLRLSCAASGRTFITYAIGWFRQAPGKEREFVTAINWSG |
| | | SMTSYADSVKGRETISRDNAKNTVYLQINGLKSEDTAVYYCAAHRGAIAPMTQSV |
| | | YDYWGQGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS11947 | 533 | DVQLVESGGGLVQAGDSLRLTCSASGRIFISYAVGWFRQAPGTEREFVAGIRWNG |
| | | ISTDYTDSVKGRFTISRDNAKNTVYLQMNSLKPEDTAIYYCAAHRTIATIPNKYE |
| | | YDHWGQGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS11948 | 534 | EVQLVESGGGLVQAGDSLRLSCVASGRTFVTYGMGWFRQAPGKEREFVAAINWSG |
| | | SMTSYGDSVKGRFAISRDNAKNTVYLQMNSLKPEDTAVYYCAAALGAVVYTTREP |
| | | YTYWGRGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLIVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS12003 | 535 | EVQLVESGGGLVQAGDSLRLSCAASGRTFLSYAVGWERQAPGTEREFVAGIRWSG |
| | | GSTDYADSVKGRETISRDNAKNTVYLQMNSLKPEDTAVYYCAAHRTIATVPNKYE |
| | | YDTWGQGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06730A | 536 | QVQLVESGGGLVQAGGSLRLSCAASGRTFITYAIGWFRQAPGKEREFVSAIAWSG |
| | | SMTSYADSVKGRFTISRDNAKNTVYLQMNGLKPEDTAVYYCAAHRGAIAPIAQSV |
| | | YTNWGQGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06730S | 537 | QVQLVESGGGLVQAGGSLRLSCAASGRIFITYAIGWERQAPGKEREFVSAISWSG |
| | | SMTSYADSVKGRFTISRDNAKNTVYLQMNGLKPEDTAVYYCAAHRGAIAPIAQSV |
| | | YTNWGQGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06730Q | 538 | QVQLVESGGGLVQAGGSLRLSCAASGRTFITYAIGWFRQAPGKEREFVSAIQWSG |
| | | SMTSYADSVKGRETISRDNAKNTVYLQMNGLKPEDTAVYYCAAHRGAIAPIAQSV |
| | | YTNWGQGTQVTVSSEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLIVDKSRWQQGN |
| | | VESCSVMHEALHNHYTQKSLSLSPGK |
| AS06730QVH1 | 539 | EVQLVESGGGLVQPGGSLRLSCAASGRIFITYAIGWVRQAPGKGLEWVSAIQWSG |
| | | SMTSYADSVKGRFTISRDNSKNTLYLQMNSLRAEDTAVYYCAKHRGAIAPIAQSV |
| | | YTNWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06730QVH2 | 540 | EVQLVESGGGLVQPGGSLRLSCAASGRTFITYAIGWERQAPGKGLEWVSAIQWSG |
| | | SMTSYADSVKGRFTISRDNSKNTVYLQMNSLRAEDTAVYYCAAHRGAIAPIAQSV |
| | | YTNWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLIVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06730QVH3a | 541 | EVQLVESGGGLVQPGGSLRLSCAASGRTFITYAIGWFRQAPGKGLEFVSAIQWSG |
| | | SMTSYADSVKGRFTISRDNSKNTVYLQMNSLRAEDTAVYYCAAHRGAIAPIAQSV |
| | | YTNWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06730SVH12 | 542 | EVQLVESGGGLVQPGGSLRLSCAASGRTFITYAIGWFRQAPGKGREFVSAISWSG |
| | | SMTSYADSVKGRETISRDNAKNTLYLQMNSLRPEDTAVYYCAAHRGAIAPIAQSV |
| | | YTNWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06730SVH12M8 | 543 | EVQLVESGGGLVQPGGSLRLSCAASGRTFITYAIGWFRQAPGKGREFVSAISWSG |
| | | SITSYADSVKGRFTISRDNAKNTLYLQMNSLRPEDTAVYYCAAHRGAIAPIAQSV |
| | | YTNWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLIVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06730SVH12M9 | 544 | EVQLVESGGGLVQPGGSLRLSCAASGRTFITYAIGWFRQAPGKGREFVSAISWSG |
| | | SLTSYADSVKGRFTISRDNAKNTLYLQMNSLRPEDTAVYYCAAHRGAIAPIAQSV |
| | | YTNWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLIVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKITPPVLDSDGSFFLYSKLTVDKSRWQQGN |
| | | VFSCSVMHEALHNHYTQKSLSLSPGK |
| AS06750VH1 | 545 | EVQLVESGGGLVQPGGSLRLSCAASGRTFLTYAVGWVRQAPGKGLEWVSGIRWSG |
| | | GYTDYADSVKGRFTISRDNSKNTLYLQMNSLRAEDTAVYYCAKHRTIATIPEKYE |
| | | YEYWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLIVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06750VH2 | 546 | EVQLVESGGGLVQPGGSLRLSCAASGRIFLTYAVGWFRQAPGKGLEWVAGIRWSG |
| | | GYTDYADSVKGRETISRDNSKNTVYLQMNSLRAEDTAVYYCAAHRTIATIPEKYE |
| | | YEYWGQGTLVTVSSEPKSCDKTHTCPPCPAPELLGGPSVELFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06750VH3 | 547 | EVQLVESGGGLVQPGGSLRLSCAASGRTFLTYAVGWFRQAPGKGLEFVAGIRWSG |
| | | GYTDYADSVKGRFTISRDNSKNTVYLQMNSLRAEDTAVYYCAAHRTIATIPEKYE |
| | | YEYWGQGTLVTVSSEPKSCDKTHTCPPCPAPELLGGPSVFLFPPKPKDTLMISRT |
| | | PEVTCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06750VHa | 548 | EVQLVESGGGLVQPGGSLRLSCTASGRTFLTYAVGWFRQAPGKGLEFVAGIRWSG |
| | | GYTDYADSVKGRFTISRDNSKNTVYLQMNSLRAEDTAVYYCAAHRTIATIPEKYE |
| | | YEYWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS06750VH11 | 549 | EVQLVESGGGLVQPGGSLRLSCAASGRTELTYAVGWFRQAPGKGREFVSGIRWSG |
| | | GYTDYADSVKGRFTISRDNAKNTLYLQMNSLRPEDTAVYYCAAHRTIATIPEKYE |
| | | YEYWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS11948A | 550 | EVQLVESGGGLVQAGDSLRLSCVASGRIFVTYGMGWFRQAPGKEREFVAAIAWSG |
| | | SMTSYGDSVKGRFAISRDNAKNTVYLQMNSLKPEDTAVYYCAAALGAVVYTTREP |
| | | YTYWGRGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLIVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS11948S | 551 | EVQLVESGGGLVQAGDSLRLSCVASGRTFVTYGMGWFRQAPGKEREFVAAISWSG |
| | | SMTSYGDSVKGRFAISRDNAKNTVYLQMNSLKPEDTAVYYCAAALGAVVYTTREP |
| | | YTYWGRGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS11948Q | 552 | EVQLVESGGGLVQAGDSLRLSCVASGRTFVTYGMGWFRQAPGKEREFVAAIQWSG |
| | | SMTSYGDSVKGRFAISRDNAKNTVYLQMNSLKPEDTAVYYCAAALGAVVYTTREP |
| | | YTYWGRGTQVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS11948QVH1 | 553 | EVQLVESGGGLVQPGGSLRLSCAASGRIFVTYGMGWVRQAPGKGLEWVSAIQWSG |
| | | SMTSYGDSVKGRETISRDNSKNTLYLQMNSLRAEDTAVYYCAKALGAVVYTTREP |
| | | YTYWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS11948QVH2 | 554 | EVQLVESGGGLVQPGGSLRLSCAASGRTFVTYGMGWFRQAPGKGLEFVAAIQWSG |
| | | SMTSYGDSVKGRFTISRDNSKNTVYLQMNSLRAEDTAVYYCAAALGAVVYTTREP |
| | | YTYWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS11948QVHa | 555 | EVQLVESGGGLVQPGGSLRLSCVASGRIFVTYGMGWFRQAPGKGREFVSAIQWSG |
| | | SMTSYGDSVKGRETISRDNSKNTVYLQMNSLRAEDTAVYYCAAALGAVVYTTREP |
| | | YTYWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS11948SVH12 | 556 | EVQLVESGGGLVQPGGSLRLSCAASGRIFVTYGMGWFRQAPGKGREFVSAISWSG |
| | | SMTSYGDSVKGRFTISRDNAKNTLYLQMNSLRPEDTAVYYCAAALGAVVYTTREP |
| | | YTYWGQGTLVTVSSEPKSCDKTHTCPPCPAPELLGGPSVELFPPKPKDTLMISRT |
| | | PEVTCVVVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS11948SVH12M8 | 557 | EVQLVESGGGLVQPGGSLRLSCAASGRTFVTYGMGWFRQAPGKGREFVSAISWSG |
| | | SITSYGDSVKGRFTISRDNAKNTLYLQMNSLRPEDTAVYYCAAALGAVVYTTREP |
| | | YTYWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |
| AS11948SVH12M9 | 558 | EVQLVESGGGLVQPGGSLRLSCAASGRTFVTYGMGWFRQAPGKGREFVSAISWSG |
| | | SLTSYGDSVKGRFTISRDNAKNTLYLQMNSLRPEDTAVYYCAAALGAVVYTTREP |
| | | YTYWGQGTLVTVSSEPKSCDKTHTCPPCPAPELLGGPSVFLFPPKPKDTLMISRT |
| | | PEVTCVVVDVSHEDPEVKENWYVDGVEVHNAKTKPREEQYNSTYRVVSVLTVLHQ |
| | | DWLNGKEYKCKVSNKALPAPIEKTISKAKGQPREPQVYTLPPSRDELTKNQVSLT |
| | | CLVKGFYPSDIAVEWESNGQPENNYKTTPPVLDSDGSFFLYSKLTVDKSRWQQGN |
| | | VFSCSVMHEALHNHYTQKSLSLSPGK |

ID: SEQ ID NO

### Example 11

### Anti-SIRPα Anti-PD-L1 Bispecific Antibodies

In this example, a few anti-SIRPα anti-PD-L1 bispecific antibodies were generated and tested, and showed potent activities in enhancing both T cell function and macrophage phagocytosis.

Bispecific antibodies of four different formats (FIG. 6A-D) were prepared with two anti-SIRPα antibodies (HSP210-02-hz52, a humanized version from 248G3F6, and HSP210-03-hz51, a humanized version from 300A6A6) and an anti-PD-L1 sdAb (AS11948SVH12, a humanized version of AS11948).

In the format illustrated in FIG. 6A, the anti-PD-L1 sdAb is located at the N-terminus of the Fc fragment of the anti-SIRPα antibody (Format I). In the format illustrated in FIG. 6B, the anti-PD-L1 sdAb is located at the C-terminus of the heavy chains of the anti-SIRPα antibody (Format II). In the format illustrated in FIG. 6C, the anti-PD-L1 sdAb is located at the C-terminus of the light chains of the anti-SIRPα antibody (Format III). In the format illustrated in FIG. 6D, the anti-PD-L1 sdAb is located at the N-terminus of the light chains of the anti-SIRPα antibody (Format IV).

TABLE 29
Listing of bispecific antibodies

| Name | Location of anti-PD-L1 sdAb |
|---|---|
| PD-L1/SIRPa-HC1 | Format I - C-terminus of IgG4 Fc (248G3F6) |
| PD-L1/SIRPa-HC2 | Format I - C-terminus of IgG4 Fc (300A6A6) |
| PD-L1/SIRPa-HN1 | Format II - N-terminus of heavy chain (248G3F6) |
| PD-L1/SIRPa-HN2 | Format II - N-terminus of heavy chain (300A6A6) |
| PD-L1/SIRPa-LC1 | Format III - C-terminus of light chain (248G3F6) |
| PD-L1/SIRPa-LC2 | Format III - C-terminus of light chain (300A6A6) |
| PD-L1/SIRPa-LN1 | Format IV - N-terminus of light chain (248G3F6) |
| PD-L1/SIRPa-LN2 | Format IV - N-terminus of light chain (300A6A6) |

The sequences of these antibodies are provided in the table below.

TABLE 30
Sequences of the bispecific antibodies

| Name | Sequence | SEQ ID NO: |
|---|---|---|
| PD-L1 sdAb | EVQLVESGGGLVQPGGSLRLSCAASGRTFVTYGMGWFRQAPGKGREFVSA | 513 |
| | ISWSGSMTSYGDSVKGRFTISRDNAKNTLYLQMNSLRPEDTAVYYCAAAL | |
| | GAVVYTTREPYTYWGQGTLVTVSS | |
| SIRPa IgG#1 02-hz52 | QVQLVQSGAEVKKPGASVKVSCKASGFNFEDTYMHWVRQAPGQGLEWMGR | 27 |
| VH1 | IDPADADTKYNPKFQDRVTITVDTSTNTAYMELSSLRSEDTAVYYCVRGN | |
| | YVNWGQGTTVTVSS | |
| SIRPa IgG#1 02-hz52 | EIVLTQSPGTLSLSPGERATLSCRASSSVSSSYLYWYQQKPGQAPRLLIY | 29 |
| VL1 | STSNLASGIPDRFSGSGSGTDYTLTISRLEPEDAAVYFCHQWYSYPRTFG | |
| | GGTKVEIK | |
| SIRPa IgG#2 03-hz51 | QVQLVQSGAEVKKPGSSVKVSCKASGYTFTSNYIHWVRQAPGQGLEWMGW | 43 |
| VH2 | IYPGDADTNYNQKFNGRVTLTADKSTSTAYMELSSLRSEDTAVYYCAINY | |
| | GGIWFAYWGQGTLVTVSS | |
| SIRPa IgG#2 03-hz51 | DIQMTQSPSSLSASVGDRVTITCQASQDIGNKLIWYQQKPGKAPKLLIYY | 45 |
| VL2 | VTNLPGGVPSRFSGSGSGTDFTLTISSLQPEDFATYYCLQYKQNPLTFGQ | |
| | GTKLEIK | |
| PD-L1/SIRPa-HC1 | QVQLVQSGAEVKKPGASVKVSCKASGFNFEDTYMHWVRQAPGQGLEWMGR | 559 |
| heavy chain | IDPADADTKYNPKFQDRVTITVDTSTNTAYMELSSLRSEDTAVYYCVRGN | |
| | YVNWGQGTTVTVSSASTKGPSVFPLAPCSRSTSESTAALGCLVKDYFPEP | |
| | VTVSWNSGALTSGVHTFPAVLQSSGLYSLSSVVTVPSSSLGTKTYTCNVD | |
| | HKPSNTKVDKRVESKYGPPCPPCPAPEFLGGPSVFLFPPKPKDTLMISRT | |
| | PEVTCVVVDVSQEDPEVQFNWYVDGVEVHNAKTKPREEQFNSTYRVVSVL | |
| | TVLHQDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDGSFFL | |
| | YSRLTVDKSRWQEGNVFSCSVMHEALHNHYTQKSLSLSLGK [symbol] | |
| | [symbol] EVQLVESGGGLVQPGGSLRLSCAASGRTFVTYGMGWFRQAPGKG | |
| | REFVSAISWSGSMTSYGDSVKGRFTISRDNAKNTLYLQMNSLRPEDTAVY | |
| | YCAAALGAVVYTTREPYTYWGQGTLVTVSS | |
| PD-L1/SIRPa-HC1 | EIVLTQSPGTLSLSPGERATLSCRASSSVSSSYLYWYQQKPGQAPRLLIY | 560 |
| light chain | STSNLASGIPDRFSGSGSGTDYTLTISRLEPEDAAVYFCHQWYSYPRTFG | |
| | GGTKVEIKRTVAAPSVFIFPPSDEQLKSGTASVVCLLNNFYPREAKVQWK | |
| | VDNALQSGNSQESVTEQDSKDSTYSLSSTLTLSKADYEKHKVYACEVTHQ | |
| | GLSSPVTKSFNRGEC | |
| PD-L1/SIRPa-HC2 | QVQLVQSGAEVKKPGSSVKVSCKASGYTFTSNYIHWVRQAPGQGLEWMGW | 561 |
| heavy chain | IYPGDADTNYNQKFNGRVTLTADKSTSTAYMELSSLRSEDTAVYYCAINY | |
| | GGIWFAYWGQGTLVTVSSASTKGPSVFPLAPCSRSTSESTAALGCLVKDY | |
| | FPEPVTVSWNSGALTSGVHTFPAVLQSSGLYSLSSVVTVPSSSLGTKTYT | |
| | CNVDHKPSNTKVDKRVESKYGPPCPPCPAPEFLGGPSVFLFPPKPKDTLM | |
| | ISRTPEVTCVVVDVSQEDPEVQFNWYVDGVEVHNAKTKPREEQFNSTYRV | |
| | VSVLTVLHQDWLNGKEYKCKVSNKGLPSSIEKTISKAKGQPREPQVYTLP | |
| | PSQEEMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK [symbol] | |
| | [symbol] EVQLVESGGGLVQPGGSLRLSCAASGRTFVTYGMGWFRQA | |
| | PGKGREFVSAISWSGSMTSYGDSVKGRFTISRDNAKNTLYLQMNSLRPED | |
| | TAVYYCAAALGAVVYTTREPYTYWGQGTLVTVSS | |
| PD-L1/SIRPa-HC2 | DIQMTQSPSSLSASVGDRVTITCQASQDIGNKLIWYQQKPGKAPKLLIYY | 562 |
| light chain | VTNLPGGVPSRFSGSGSGTDFTLTISSLQPEDFATYYCLQYKQNPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PD-L1/SIRPa-HN1 | EVQLVESGGGLVQPGGSLRLSCAASGRTFVTYGMGWFRQAPGKGREFVSA | 563 |
| heavy chain | ISWSGSMTSYGDSVKGRFTISRDNAKNTLYLQMNSLRPEDTAVYYCAAAL | |
| | GAVVYTTREPYTYWGQGTLVTVSS [symbol] QVQLVQSGAEV | |
| | KKPGASVKVSCKASGFNFEDTYMHWVRQAPGQGLEWMGRIDPADADTKYN | |
| | PKFQDRVTITVDTSTNTAYMELSSLRSEDTAVYYCVRGNYVNWGQGTTVT | |
| | VSSASTKGPSVFPLAPCSRSTSESTAALGCLVKDYFPEPVTVSWNSGALT | |
| | SGVHTFPAVLQSSGLYSLSSVVTVPSSSLGTKTYTCNVDHKPSNTKVDKR | |
| | VESKYGPPCPPCPAPEFLGGPSVFLFPPKPKDTLMISRTPEVTCVVVDVS | |
| | QEDPEVQFNWYVDGVEVHNAKTKPREEQFNSTYRVVSVLTVLHQDWLNGK | |
| | EYKCKVSNKGLPSSIEKTISKAKGQPREPQVYTLPPSQEEMTKNQVSLTC | |
| | LVKGFYPSDIAVEWESNGQPENNYKTTPPVLDSDGSFFLYSRLTVDKSRW | |
| | QEGNVFSCSVMHEALHNHYTQKSLSLSLGK | |
| PD-L1/SIRPa-HN1 | EIVLTQSPGTLSLSPGERATLSCRASSSVSSSYLYWYQQKPGQAPRLLIY | 560 |
| light chain | STSNLASGIPDRFSGSGSGTDYTLTISRLEPEDAAVYFCHQWYSYPRTFG | |
| | GGTKVEIKRTVAAPSVFIFPPSDEQLKSGTASVVCLLNNFYPREAKVQWK | |
| | VDNALQSGNSQESVTEQDSKDSTYSLSSTLTLSKADYEKHKVYACEVTHQ | |
| | GLSSPVTKSFNRGEC | |
| PD-L1/SIRPa-HN2 | EVQLVESGGGLVQPGGSLRLSCAASGRTFVTYGMGWFRQAPGKGREFVSA | 564 |
| heavy chain | ISWSGSMTSYGDSVKGRFTISRDNAKNTLYLQMNSLRPEDTAVYYCAAAL | |
| | GAVVYTTREPYTYWGQGTLVTVSS [symbol] QVQLVQSGAEV | |
| | KKPGSSVKVSCKASGYTFTSNYIHWVRQAPGQGLEWMGWIYPGDADTNYN | |
| | QKFNGRVTLTADKSTSTAYMELSSLRSEDTAVYYCAINYGGIWFAYWGQG | |
| | TLVTVSSASTKGPSVFPLAPCSRSTSESTAALGCLVKDYFPEPVTVSWNS | |
| | GALTSGVHTFPAVLQSSGLYSLSSVVTVPSSSLGTKTYTCNVDHKPSNTK | |
| | VDKRVESKYGPPCPPCPAPEFLGGPSVFLFPPKPKDTLMISRTPEVTCVV | |
| | VDVSQEDPEVQFNWYVDGVEVHNAKTKPREEQFNSTYRVVSVLTVLHQDW | |
| | LNGKEYKCKVSNKGLPSSIEKTISKAKGQPREPQVYTLPPSQEEMTKNQV | |
| | SLTCLVKGFYPSDIAVEWESNGQPENNYKTTPPVLDSDGSFFLYSRLTVD | |
| | KSRWQEGNVFSCSVMHEALHNHYTQKSLSLSLGK | |
| PD-L1/SIRPa-HN2 | DIQMTQSPSSLSASVGDRVTITCQASQDIGNKLIWYQQKPGKAPKLLIYY | 562 |
| light chain | VTNLPGGVPSRFSGSGSGTDFTLTISSLQPEDFATYYCLQYKQNPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC | |
| PD-L1/SIRPa-LC1 | QVQLVQSGAEVKKPGASVKVSCKASGFNFEDTYMHWVRQAPGQGLEWMGR | 565 |
| heavy chain | IDPADADTKYNPKFQDRVTITVDTSTNTAYMELSSLRSEDTAVYYCVRGN | |
| | YVNWGQGTTVTVSSASTKGPSVFPLAPCSRSTSESTAALGCLVKDYFPEP | |
| | VTVSWNSGALTSGVHTFPAVLQSSGLYSLSSVVTVPSSSLGTKTYTCNVD | |
| | HKPSNTKVDKRVESKYGPPCPPCPAPEFLGGPSVFLFPPKPKDTLMISRT | |
| | PEVTCVVVDVSQEDPEVQFNWYVDGVEVHNAKTKPREEQFNSTYRVVSVL | |
| | TVLHQDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDGSFFL | |
| | YSRLTVDKSRWQEGNVFSCSVMHEALHNHYTQKSLSLSLGK | |
| PD-L1/SIRPa-LC1 | EIVLTQSPGTLSLSPGERATLSCRASSSVSSSYLYWYQQKPGQAPRLLIY | 566 |
| light chain | STSNLASGIPDRFSGSGSGTDYTLTISRLEPEDAAVYFCHQWYSYPRTFG | |
| | GGTKVEIKRTVAAPSVFIFPPSDEQLKSGTASVVCLLNNFYPREAKVQWK | |
| | VDNALQSGNSQESVTEQDSKDSTYSLSSTLTLSKADYEKHKVYACEVTHQ | |
| | GLSSPVTKSFNRGEC [symbol] EVQLVESGGGLVQPGGSLRL | |
| | SCAASGRTFVTYGMGWFRQAPGKGREFVSAISWSGSMTSYGDSVKGRFTI | |
| | SRDNAKNTLYLQMNSLRPEDTAVYYCAAALGAVVYTTREPYTYWGQGTLV | |
| | TVSS | |
| PD-L1/SIRPa-LC2 | QVQLVQSGAEVKKPGSSVKVSCKASGYTFTSNYIHWVRQAPGQGLEWMGW | 567 |
| heavy chain | IYPGDADTNYNQKFNGRVTLTADKSTSTAYMELSSLRSEDTAVYYCAINY | |
| | GGIWFAYWGQGTLVTVSSASTKGPSVFPLAPCSRSTSESTAALGCLVKDY | |
| | FPEPVTVSWNSGALTSGVHTFPAVLQSSGLYSLSSVVTVPSSSLGTKTYT | |
| | CNVDHKPSNTKVDKRVESKYGPPCPPCPAPEFLGGPSVFLFPPKPKDTLM | |
| | ISRTPEVTCVVVDVSQEDPEVQFNWYVDGVEVHNAKTKPREEQFNSTYRV | |
| | VSVLTVLHQDWLNGKEYKCKVSNKGLPSSIEKTISKAKGQPREPQVYTLP | |
| | PSQEEMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| PD-L1/SIRPa-LC2 | DIQMTQSPSSLSASVGDRVTITCQASQDIGNKLIWYQQKPGKAPKLLIYY | 568 |
| light chain | VTNLPGGVPSRFSGSGSGTDFTLTISSLQPEDFATYYCLQYKQNPLTFGQ | |
| | GTKLEIKRTVAAPSVFIFPPSDEQLKSGTASVVCLLNNFYPREAKVQWKV | |
| | DNALQSGNSQESVTEQDSKDSTYSLSSTLTLSKADYEKHKVYACEVTHQG | |
| | LSSPVTKSFNRGEC [symbol] EVQLVESGGGLVQPGGSLRLS | |
| | CAASGRTFVTYGMGWFRQAPGKGREFVSAISWSGSMTSYGDSVKGRFTIS | |
| | RDNAKNTLYLQMNSLRPEDTAVYYCAAALGAVVYTTREPYTYWGQGTLVT | |
| | VSS | |
| PD-L1/SIRPa-LN1 | QVQLVQSGAEVKKPGASVKVSCKASGFNFEDTYMHWVRQAPGQGLEWMGR | 565 |
| heavy chain | IDPADADTKYNPKFQDRVTITVDTSTNTAYMELSSLRSEDTAVYYCVRGN | |
| | YVNWGQGTTVTVSSASTKGPSVFPLAPCSRSTSESTAALGCLVKDYFPEP | |
| | VTVSWNSGALTSGVHTFPAVLQSSGLYSLSSVVTVPSSSLGTKTYTCNVD | |
| | HKPSNTKVDKRVESKYGPPCPPCPAPEFLGGPSVFLFPPKPKDTLMISRT | |
| | PEVTCVVVDVSQEDPEVQFNWYVDGVEVHNAKTKPREEQFNSTYRVVSVL | |
| | TVLHQDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDGSFFL | |
| | YSRLTVDKSRWQEGNVFSCSVMHEALHNHYTQKSLSLSLGK | |
| PD-L1/SIRPa-LN1 | EVQLVESGGGLVQPGGSLRLSCAASGRTFVTYGMGWFRQAPGKGREFVSA | 569 |
| light chain | ISWSGSMTSYGDSVKGRFTISRDNAKNTLYLQMNSLRPEDTAVYYCAAAL | |
| | GAVVYTTREPYTYWGQGTLVTVSS [symbol] EIVLTQSPGTL | |
| | SLSPGERATLSCRASSSVSSSYLYWYQQKPGQAPRLLIYSTSNLASGIPD | |
| | RFSGSGSGTDYTLTISRLEPEDAAVYFCHQWYSYPRTFGGGTKVEIKRTV | |
| | AAPSVFIFPPSDEQLKSGTASVVCLLNNFYPREAKVQWKVDNALQSGNSQ | |
| | ESVTEQDSKDSTYSLSSTLTLSKADYEKHKVYACEVTHQGLSSPVTKSFN | |
| | RGEC | |
| PD-L1/SIRPa-LN2 | QVQLVQSGAEVKKPGSSVKVSCKASGYTFTSNYIHWVRQAPGQGLEWMGW | 567 |
| heavy chain | IYPGDADTNYNQKFNGRVTLTADKSTSTAYMELSSLRSEDTAVYYCAINY | |
| | GGIWFAYWGQGTLVTVSSASTKGPSVFPLAPCSRSTSESTAALGCLVKDY | |
| | FPEPVTVSWNSGALTSGVHTFPAVLQSSGLYSLSSVVTVPSSSLGTKTYT | |
| | CNVDHKPSNTKVDKRVESKYGPPCPPCPAPEFLGGPSVFLFPPKPKDTLM | |
| | ISRTPEVTCVVVDVSQEDPEVQFNWYVDGVEVHNAKTKPREEQFNSTYRV | |
| | VSVLTVLHQDWLNGKEYKCKVSNKGLPSSIEKTISKAKGQPREPQVYTLP | |
| | PSQEEMTKNQVSLTCLVKGFYPSDIAVEWESNGQPENNYKTTPPVLDSDG | |
| | SFFLYSRLTVDKSRWQEGNVFSCSVMHEALHNHYTQKSLSLSLGK | |
| PD-L1/SIRPa-LN2 | EVQLVESGGGLVQPGGSLRLSCAASGRTFVTYGMGWFRQAPGKGREFVSA | 570 |
| light chain | ISWSGSMTSYGDSVKGRFTISRDNAKNTLYLQMNSLRPEDTAVYYCAAAL | |
| | GAVVYTTREPYTYWGQGTLVTVSS [symbol] DIQMTQSPSSL | |
| | SASVGDRVTITCQASQDIGNKLIWYQQKPGKAPKLLIYYVTNLPGGVPSR | |
| | FSGSGSGTDFTLTISSLQPEDFATYYCLQYKQNPLTFGQGTKLEIKRTVA | |
| | APSVFIFPPSDEQLKSGTASVVCLLNNFYPREAKVQWKVDNALQSGNSQE | |
| | SVTEQDSKDSTYSLSSTLTLSKADYEKHKVYACEVTHQGLSSPVTKSFNR | |
| | GEC | |

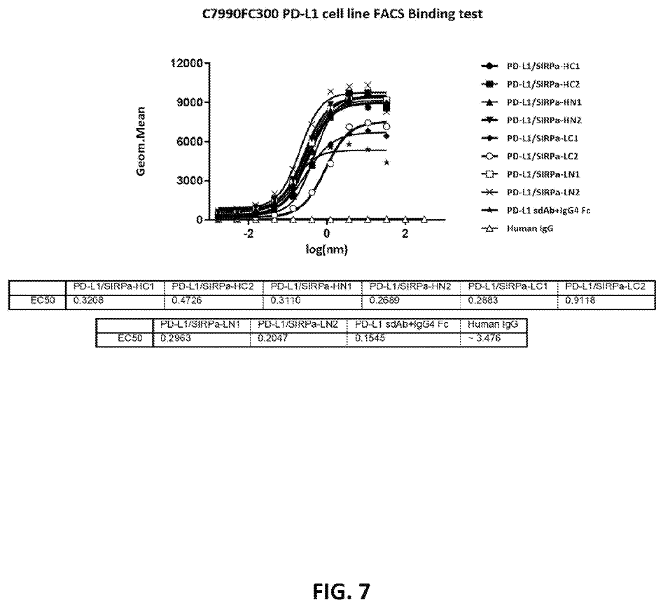

The bispecific antibodies were first tested for their ability to bind to PD-L1 expressed on cells. The results are summarized in FIG. 7, which shows that all of the tested bispecific antibodies had stronger affinity than IgG and anti-PD-L1 sdAb alone (fused to IgG4 Fc).

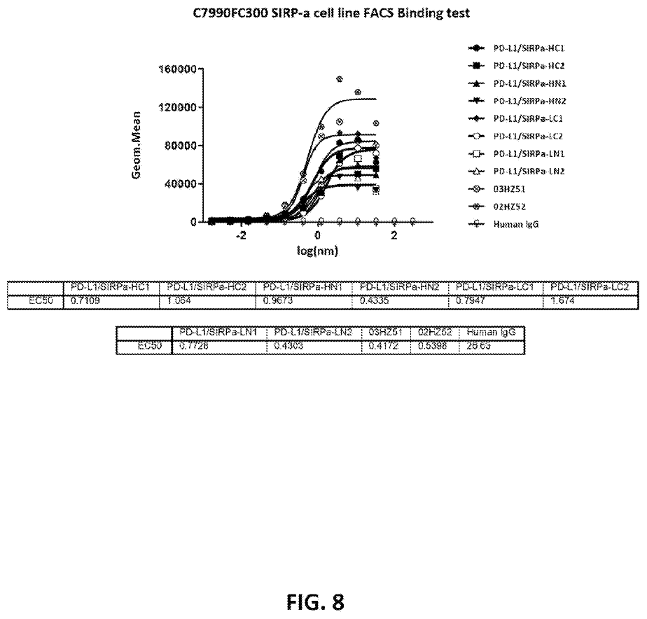

The bispecific antibodies were also tested for their ability to bind to SIRP-α expressed on cells. As shown in FIG. 8, the tested bispecific antibodies had comparable affinity to the monospecific counterparts (03HZ51: HSP210-03-hz51, 02HZ52: HSP210-02-hz52).

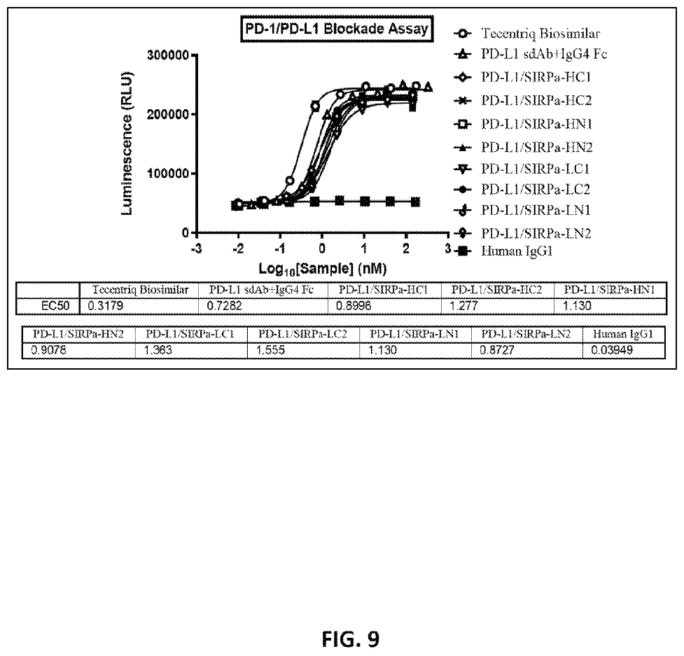

The bispecific antibodies' activity in blocking PD-1/PD-L1 interaction was also tested and the results are shown in FIG. 9. All of them exhibited superior biological activities, which are comparable to Tecentriq®.

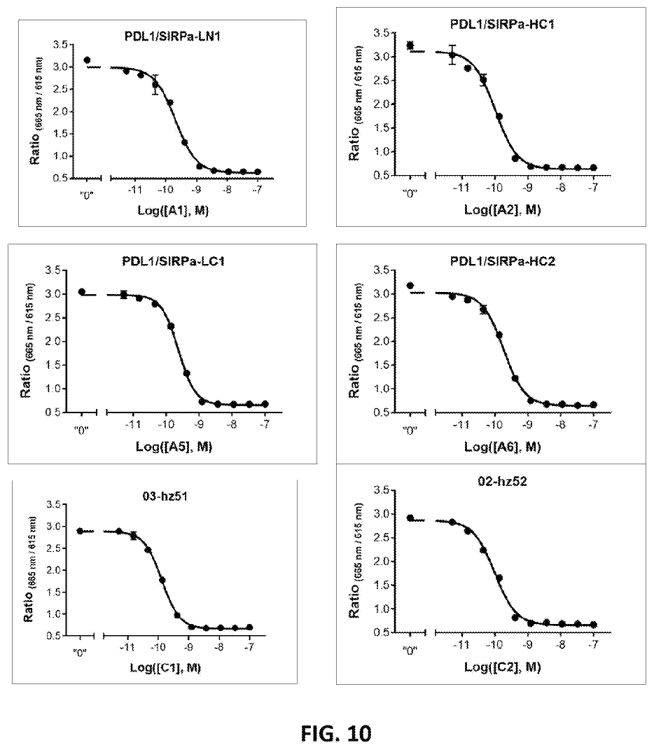

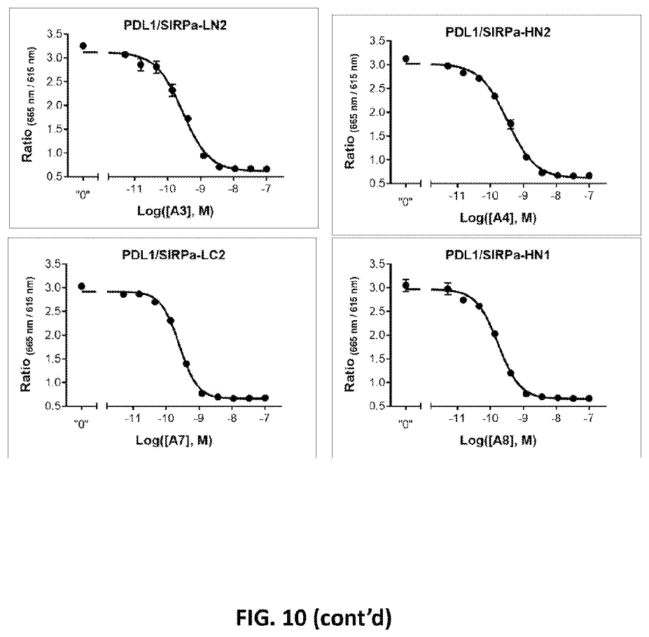

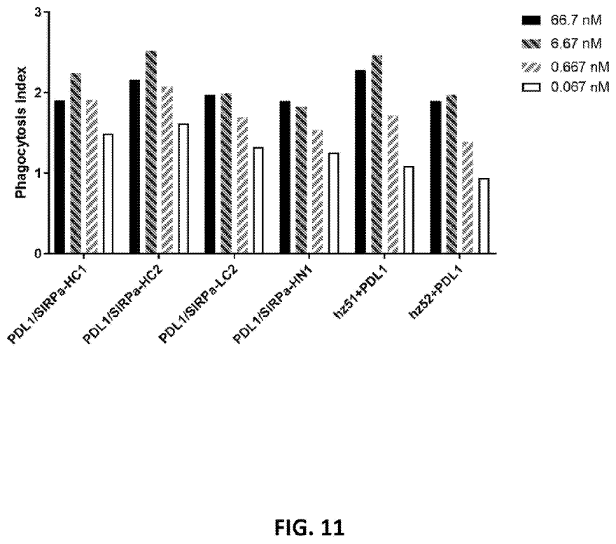

The bispecific antibodies' activity in blocking CD47/SIRPα interaction was further tested and the results are shown in FIG. 10. All of them exhibited superior biological activities, which are comparable to parent monocolonal SIRPα antibodies. Four of the bispecific antibodies (PD-L1/SIRPα-HC1, PD-L1/SIRPα-HC2, PD-L1/SIRPα-LC2, PD-L1/SIRPα-LN1) were selected for testing with respect their ability to induce phagocytosis. The procedure is similar to Example 7, and the results are summarized in FIG. 11. All of these bispecific antibodies exhibited similar activities as the monospecific counterparts, and PD-L1/SIRPα-HC1 and PD-L1/SIRPα-HC2, wherein the sdAb was fused to the C-terminus of the Fc fragment, showed slightly stronger activity.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An antibody, comprising an anti-signal regulatory protein alpha (SIRPα) unit and an anti-programmed death-ligand 1 (PD-L1) unit, wherein the anti-SIRPα unit comprises an Fab fragment having binding specificity to a human SIRPα protein, and the anti-PD-L1 unit comprises a single-domain antibody (sdAb) having binding specificity to a human PD-L1 protein.

2. The antibody of claim 1, further comprising an Fc fragment.

3. The antibody of claim 1, wherein the sdAb is fused to the heavy chain of the Fab fragment.

4. The antibody of claim 1, wherein the sdAb is fused to the C-terminus of the heavy chain.

5. The antibody of claim 1, wherein the Fab fragment comprises a heavy chain variable region comprising heavy chain complementarity determining regions CDRH1, CDRH2, and CDRH3 and a light chain variable region light chain comprising complementarity determining regions CDRL1, CDRL2, and CDRL3, and wherein:
(a) the CDRH1 comprises the amino acid sequence of SEQ ID NO: 15, the CDRH2 comprises the amino acid sequence of SEQ ID NO: 16, 21 or 22, the CDRH3 comprises the amino acid sequence of SEQ ID NO: 17, the CDRL1 comprises the amino acid sequence of SEQ ID NO: 18, the CDRL2 comprises the amino acid sequence of SEQ ID NO: 19, and the CDRL3 comprises the amino acid sequence of SEQ ID NO: 20;
(b) the CDRH1 comprises the amino acid sequence of SEQ ID NO: 30, the CDRH2 comprises the amino acid sequence of SEQ ID NO: 31, 36, 37 or 38, the CDRH3 comprises the amino acid sequence of SEQ ID NO: 32, the CDRL1 comprises the amino acid sequence of SEQ ID NO: 33, the CDRL2 comprises the amino acid sequence of SEQ ID NO: 34, and the CDRL3 comprises the amino acid sequence of SEQ ID NO: 35;
(c) the CDRH1 comprises the amino acid sequence of SEQ ID NO: 47, the CDRH2 comprises the amino acid sequence of SEQ ID NO: 48, 53 or 54, the CDRH3 comprises the amino acid sequence of SEQ ID NO: 49, the CDRL1 comprises the amino acid sequence of SEQ ID NO: 50, the CDRL2 comprises the amino acid sequence of SEQ ID NO: 51, and the CDRL3 comprises the amino acid sequence of SEQ ID NO: 52;
(d) the CDRH1 comprises the amino acid sequence of SEQ ID NO: 63, the CDRH2 comprises the amino acid sequence of SEQ ID NO: 64, the CDRH3 comprises the amino acid sequence of SEQ ID NO: 65, the CDRL1 comprises the amino acid sequence of SEQ ID NO: 66, the CDRL2 comprises the amino acid sequence of SEQ ID NO: 67, and the CDRL3 comprises the amino acid sequence of SEQ ID NO: 68;
(e) the CDRH1 comprises the amino acid sequence of SEQ ID NO: 77, the CDRH2 comprises the amino acid sequence of SEQ ID NO: 78, the CDRH3 comprises the amino acid sequence of SEQ ID NO: 79, the CDRL1 comprises the amino acid sequence of SEQ ID NO: 80, the CDRL2 comprises the amino acid sequence of SEQ ID NO: 81, and the CDRL3 comprises the amino acid sequence of SEQ ID NO: 82;
(f) the CDRH1 comprises the amino acid sequence of SEQ ID NO: 91, the CDRH2 comprises the amino acid sequence of SEQ ID NO: 92, the CDRH3 comprises the amino acid sequence of SEQ ID NO: 93, the CDRL1 comprises the amino acid sequence of SEQ ID NO: 94, the CDRL2 comprises the amino acid sequence of SEQ ID NO: 95, and the CDRL3 comprises the amino acid sequence of SEQ ID NO: 96; or
(g) the CDRH1 comprises the amino acid sequence of SEQ ID NO: 103, the CDRH2 comprises the amino acid sequence of SEQ ID NO: 104, the CDRH3 comprises the amino acid sequence of SEQ ID NO: 105, the CDRL1 comprises the amino acid sequence of SEQ ID NO: 106, the CDRL2 comprises the amino acid sequence of SEQ ID NO: 107, and the CDRL3 comprises the amino acid sequence of SEQ ID NO: 108.

6. The antibody of claim 5, wherein the CDRH1 comprises the amino acid sequence of SEQ ID NO: 15, the CDRH2 comprises the amino acid sequence of SEQ ID NO: 16, 21 or 22, the CDRH3 comprises the amino acid sequence of SEQ ID NO: 17, the CDRL1 comprises the amino acid sequence of SEQ ID NO: 18, the CDRL2 comprises the amino acid sequence of SEQ ID NO: 19, and the CDRL3 comprises the amino acid sequence of SEQ ID NO: 20.

7. The antibody of claim 6, wherein the heavy chain variable region comprises an amino acid sequence selected from the group consisting of SEQ ID NO:1 and 23-27 and the light chain variable region comprises an amino acid sequence selected from the group consisting of SEQ ID NO:2 and 28-29.

8. The antibody of claim 6, wherein the heavy chain variable region comprises the amino acid sequence of SEQ ID NO:27 and the light chain variable region comprises the amino acid sequence of SEQ ID NO:29.

9. The antibody of claim 5, wherein the CDRH1 comprises the amino acid sequence of SEQ ID NO: 30, the CDRH2 comprises the amino acid sequence of SEQ ID NO: 31, 36, 37 or 38, the CDRH3 comprises the amino acid sequence of SEQ ID NO: 32, the CDRL1 comprises the amino acid sequence of SEQ ID NO: 33, the CDRL2 comprises the amino acid sequence of SEQ ID NO: 34, and the CDRL3 comprises the amino acid sequence of SEQ ID NO: 35.

10. The antibody of claim 9, wherein the heavy chain variable region comprises an amino acid sequence selected from the group consisting of SEQ ID NO:3 and 39-44 and the light chain variable region comprises an amino acid sequence selected from the group consisting of SEQ ID NO:4 and 45-46.

11. The antibody of claim 9, wherein the heavy chain variable region comprises the amino acid sequence of SEQ ID NO:43 and the light chain variable region comprises the amino acid sequence of SEQ ID NO:45.

12. The antibody of claim 5, wherein the CDRH1 comprises the amino acid sequence of SEQ ID NO: 47, the CDRH2 comprises the amino acid sequence of SEQ ID NO: 48, 53 or 54, the CDRH3 comprises the amino acid sequence of SEQ ID NO: 49, the CDRL1 comprises the amino acid sequence of SEQ ID NO: 50, the CDRL2 comprises the amino acid sequence of SEQ ID NO: 51, and the CDRL3 comprises the amino acid sequence of SEQ ID NO: 52;

13. The antibody of claim 12, wherein the heavy chain variable region comprises an amino acid sequence selected from the group consisting of SEQ ID NO:5 and 55-60 and the light chain variable region comprises an amino acid sequence selected from the group consisting of SEQ ID NO:6 and 61-62.

14. The antibody of claim 1, wherein the sdAb comprises a CDR1 comprising the amino acid sequence of any one of SEQ ID NO:169-218, a CDR2 comprising the amino acid sequence of any one of SEQ ID NO:169-318, and a CDR3 comprising the amino acid sequence of any one of SEQ ID NO:369-418.

15. The antibody of claim 1, wherein the sdAb comprises the CDR1, CDR2 and CDR3 of the amino acid sequences of SEQ ID NO:469-518.

16. The antibody of claim 14, wherein the CDR1 comprises the amino acid sequence of SEQ ID NO:213, the CDR2 comprises the amino acid sequence of SEQ ID NO:313, and the CDR3 comprises the amino acid sequence of SEQ ID NO:413.

17. The antibody of claim 14, wherein sdAb comprises the amino acid sequence of SEQ ID NO:513.

18. The antibody of claim 1, which comprises:
(a) a heavy chain comprising the amino acid sequence of SEQ ID NO:559 and a light chain comprising the amino acid sequence of SEQ ID NO:560;
(b) a heavy chain comprising the amino acid sequence of SEQ ID NO:561 and a light chain comprising the amino acid sequence of SEQ ID NO:562;
(c) a heavy chain comprising the amino acid sequence of SEQ ID NO:563 and a light chain comprising the amino acid sequence of SEQ ID NO:560;
(d) a heavy chain comprising the amino acid sequence of SEQ ID NO:564 and a light chain comprising the amino acid sequence of SEQ ID NO:562;
(e) a heavy chain comprising the amino acid sequence of SEQ ID NO:565 and a light chain comprising the amino acid sequence of SEQ ID NO:566;
(f) a heavy chain comprising the amino acid sequence of SEQ ID NO:567 and a light chain comprising the amino acid sequence of SEQ ID NO:568;
(g) a heavy chain comprising the amino acid sequence of SEQ ID NO:565 and a light chain comprising the amino acid sequence of SEQ ID NO:569; or
(h) a heavy chain comprising the amino acid sequence of SEQ ID NO:567 and a light chain comprising the amino acid sequence of SEQ ID NO:570.

19. (canceled)

20. A method of treating cancer in a patient in need thereof, comprising administering to the patient the antibody of claim 1.

21. (canceled)

22. The method of claim 20, wherein the cancer is selected from the group consisting of bladder cancer, liver cancer, colon cancer, rectal cancer, endometrial cancer, leukemia, lymphoma, pancreatic cancer, small cell lung cancer, non-small cell lung cancer, breast cancer, urethral cancer, head and neck cancer, gastrointestinal cancer, stomach cancer, oesophageal cancer, ovarian cancer, renal cancer, melanoma, prostate cancer and thyroid cancer.


SEQUENCE LISTING (570 sequences)

SEQ ID NO: 1 - protein, 114 aa, Artificial Sequence
  Synthetic
EVQLQQSGAELVKPGASVKLSCTASGFNFEDTYMHWVKQRPDQGLEWIGRIDPADGDTKYNPKFQDKATITVDTSSNTAYLQLSSLTSEDTAVYYCVRGNYVNWGQGTTLTVSS

SEQ ID NO: 2 - protein, 108 aa, Artificial Sequence
  Synthetic
QIVLIQSPAIMSASPGERVTLTCRASSSVSSSYLYWYQQKPGSSPKLWIYSTSNLASGVPARFSGSGSGTSYSLTISSMEAEDAASYFCHQWYSYPRTFGGGTKLEIK

SEQ ID NO: 3 - protein, 118 aa, Artificial Sequence
  Synthetic
QVQLQQSGTELVKPGSSVKISCKASGYTFTSNYIHWIRQQPGNGLEWIGWIYPGDGDTNYNQKFNGKATLTADKSSSTAYMQLSSLTSEDYAVYFCAINYGGIWFAYWGQGTLVTVSS

SEQ ID NO: 4 - protein, 107 aa, Artificial Sequence
  Synthetic
DIQMTQSPSSMSASLGDRVTITCQASQDIGNKLIWFQQKPGKSPRLMIHYVTNLPGGVPLRFSGSRSGSDYSLTISSLESEDMADYYCLQYKQNPLTFGSGTKLEIK

SEQ ID NO: 5 - protein, 123 aa, Artificial Sequence
  Synthetic
QVTLKESGPGILQPSQTLSLTCSFSGFSLNTYDIGMGWIRQPSGKGLEWLAHIWWNDREYYNSALQSRVTISKDTSNTQVFLKIASVDTADTATYYCVRIDYFGSGQAWFTYWGQGTLVTVSA

SEQ ID NO: 6 - protein, 108 aa, Artificial Sequence
  Synthetic
EIVLTQSPPTMAASPGEKITITCSSSSTISSTYLHWYQQKPGFSPKLLISGTSNLASGVPPRFSGSGSGTSYSLTIGTLEAEDVATYYCQQGSRIPFTFGSGTKLEIK

SEQ ID NO: 7 - protein, 113 aa, Artificial Sequence
  Synthetic
EVQLQQSGAELVKPGASVKLSCTASGFNIKDYYMHWVKQRTEQGLEWIGRIDPEDGETKYAPKFQGKATITADTSSNTAYLQLSSLTSEDTAVYYCSRSWAYWGQGTTLTVSS

SEQ ID NO: 8 - protein, 108 aa, Artificial Sequence
  Synthetic
QIVLTQSPAIMSASPGEKVTLTCSASSSVSSSYLYWYQQKPGSSPKLWIYSTSNLASGVPARFSGSGSGTSYSLTISSMEAEDAASYFCHQWSSYPRTFGGGTKLEIK

SEQ ID NO: 9 - protein, 120 aa, Artificial Sequence
  Synthetic
EVQLQQSGAELVKPGASVKLSCTASGFNIKDTYMHWVKQRPEQGLEWVGRIDPANVNTIYDPKFQGKATITADTSSNTAYLQLSSLTSEDTAVYYCARVGAYDGYDFDYWGQGTTLTVSS

SEQ ID NO: 10 - protein, 111 aa, Artificial Sequence
  Synthetic
DIVLTQSPASLAVSLGQRATISCRASESVDNYGNSFMHWYQQKPGQPPKLLIYRASNLESGIPARFSGSGSRTDFTLTINPVEADDVATYYCQQNNEDPLTFGAGTKLELK

SEQ ID NO: 11 - protein, 120 aa, Artificial Sequence
  Synthetic
EVQLQQSGPELVKPGASVKMSCKASGYTFTSYVMHWVKQKPGQGLEWIGYINPYNDGTKYNEKFKGKATLTSDKSSSTAYMELSSLTSEDSAVYYCARSYYDYDGSFDYWGQGTTLTVSS

SEQ ID NO: 12 - protein, 107 aa, Artificial Sequence
  Synthetic
DIVMTQSHKFMSTSVGDRVSITCKASQDVTTAVAWYQQKSGQSPKLLIYSASYRYTGDPDRFTGSGSGTDFTFTISSVQAEDLAVYYCQQHYSTPWTFGGGTKLEIK

SEQ ID NO: 13 - protein, 114 aa, Artificial Sequence
  Synthetic
EVQLQQSGAELVKPGASVKLSCTASGFNFEDTYIHWVKQRPDQGLEWIGRIDPADGDTKHNPKFHDKATVTVDTSSNTAYLELSSLTSEDTAVYYCVRGNYVNWGQGTTLTVSS

SEQ ID NO: 14 - protein, 108 aa, Artificial Sequence
  Synthetic
QIVLIQSPAIMSASPGERVTLTCRASSSVTSSYLYWYQQKPGSSPKLWIYSASNLASGVPARFSGSGSGTSYSLTISSVEAEDAASYFCHQWYSYPRTFGGGTKLEIK

SEQ ID NO: 15 - protein, 5 aa, Artificial Sequence
  Synthetic
DTYMH

SEQ ID NO: 16 - protein, 17 aa, Artificial Sequence
  Synthetic
RIDPADGDTKYNPKFQD

SEQ ID NO: 17 - protein, 5 aa, Artificial Sequence
  Synthetic
GNYVN

SEQ ID NO: 18 - protein, 12 aa, Artificial Sequence
  Synthetic
RASSSVSSSYLY

SEQ ID NO: 19 - protein, 7 aa, Artificial Sequence
  Synthetic
STSNLAS

SEQ ID NO: 20 - protein, 9 aa, Artificial Sequence
  Synthetic
HQWYSYPRT

SEQ ID NO: 21 - protein, 17 aa, Artificial Sequence
  Synthetic
RIDPAEGDTKYNPKFQD

SEQ ID NO: 22 - protein, 17 aa, Artificial Sequence
  Synthetic
RIDPADADTKYNPKFQD

SEQ ID NO: 23 - protein, 114 aa, Artificial Sequence
  Synthetic
QVQLVQSGAEVKKPGASVKVSCKASGFNFEDTYMHWVRQAPGQGLEWMGRIDPADGDTKYNPKFQDRVTMTRDTSTSTVYMELSSLRSEDTAVYYCARGNYVNWGQGTTVTVSS

SEQ ID NO: 24 - protein, 114 aa, Artificial Sequence
  Synthetic
QVQLVQSGAEVKKPGASVKVSCKASGFNFEDTYMHWVRQAPGQGLEWMGRIDPADGDTKYNPKFQDRVTMTVDTSTNTAYMELSSLRSEDTAVYYCVRGNYVNWGQGTTVTVSS

SEQ ID NO: 25 - protein, 114 aa, Artificial Sequence
  Synthetic
QVQLVQSGAEVKKPGASVKVSCKASGFNFEDTYMHWVRQAPGQGLEWMGRIDPADGDTKYNPKFQDRVTITVDTSTNTAYMELSSLRSEDTAVYYCVRGNYVNWGQGTTVTVSS

SEQ ID NO: 26 - protein, 114 aa, Artificial Sequence
  Synthetic
QVQLVQSGAEVKKPGASVKVSCKASGFNFEDTYMHWVRQAPGQGLEWMGRIDPAEGDTKYNPKFQDRVTITVDTSTNTAYMELSSLRSEDTAVYYCVRGNYVNWGQGTTVTVSS

SEQ ID NO: 27 - protein, 114 aa, Artificial Sequence
  Synthetic
QVQLVQSGAEVKKPGASVKVSCKASGFNFEDTYMHWVRQAPGQGLEWMGRIDPADADTKYNPKFQDRVTITVDTSTNTAYMELSSLRSEDTAVYYCVRGNYVNWGQGTTVTVSS

SEQ ID NO: 28 - protein, 108 aa, Artificial Sequence
  Synthetic
EIVLTQSPGTLSLSPGERATLSCRASSSVSSSYLYWYQQKPGQAPRLLIYSTSNLASGIPDRFSGSGSGTDFTLTISRLEPEDFAVYYCHQWYSYPRTFGGGTKVEIK

SEQ ID NO: 29 - protein, 108 aa, Artificial Sequence
  Synthetic
EIVLTQSPGTLSLSPGERATLSCRASSSVSSSYLYWYQQKPGQAPRLLIYSTSNLASGIPDRFSGSGSGTDYTLTISRLEPEDAAVYFCHQWYSYPRTFGGGTKVEIK

SEQ ID NO: 30 - protein, 5 aa, Artificial Sequence
  Synthetic
SNYIH

SEQ ID NO: 31 - protein, 17 aa, Artificial Sequence
  Synthetic
WIYPGDGDTNYNQKFNG

SEQ ID NO: 32 - protein, 9 aa, Artificial Sequence
  Synthetic
NYGGIWFAY

SEQ ID NO: 33 - protein, 11 aa, Artificial Sequence
  Synthetic
QASQDIGNKLI

SEQ ID NO: 34 - protein, 7 aa, Artificial Sequence
  Synthetic
YVTNLPG

SEQ ID NO: 35 - protein, 9 aa, Artificial Sequence
  Synthetic
LQYKQNPLT

SEQ ID NO: 36 - protein, 17 aa, Artificial Sequence
  Synthetic
WIYPGEGDTNYNQKFNG

SEQ ID NO: 37 - protein, 17 aa, Artificial Sequence
  Synthetic
WIYPGDADTNYNQKFNG

SEQ ID NO: 38 - protein, 17 aa, Artificial Sequence
  Synthetic
WIYPGDGDTNYNQKFQG

SEQ ID NO: 39 - protein, 118 aa, Artificial Sequence
  Synthetic
QVQLVQSGAEVKKPGSSVKVSCKASGYTFTSNYIHWVRQAPGQGLEWMGWIYPGDGDTNYNQKFNGRVTITADKSTSTAYMELSSLRSEDTAVYYCARNYGGIWFAYWGQGTLVTVSS

SEQ ID NO: 40 - protein, 118 aa, Artificial Sequence
  Synthetic
QVQLVQSGAEVKKPGSSVKVSCKASGYTFTSNYIHWVRQAPGQGLEWMGWIYPGDGDTNYNQKFNGRVTITADKSTSTAYMELSSLRSEDTAVYYCAINYGGIWFAYWGQGTLVTVSS

SEQ ID NO: 41 - protein, 118 aa, Artificial Sequence
  Synthetic
QVQLVQSGAEVKKPGSSVKVSCKASGYTFTSNYIHWVRQAPGQGLEWMGWIYPGDGDTNYNQKFNGRVTLTADKSTSTAYMELSSLRSEDTAVYYCAINYGGIWFAYWGQGTLVTVSS

SEQ ID NO: 42 - protein, 118 aa, Artificial Sequence
  Synthetic
QVQLVQSGAEVKKPGSSVKVSCKASGYTFTSNYIHWVRQAPGQGLEWMGWIYPGEGDTNYNQKFNGRVTLTADKSTSTAYMELSSLRSEDTAVYYCAINYGGIWFAYWGQGTLVTVSS

SEQ ID NO: 43 - protein, 118 aa, Artificial Sequence
  Synthetic
QVQLVQSGAEVKKPGSSVKVSCKASGYTFTSNYIHWVRQAPGQGLEWMGWIYPGDADTNYNQKFNGRVTLTADKSTSTAYMELSSLRSEDTAVYYCAINYGGIWFAYWGQGTLVTVSS

SEQ ID NO: 44 - protein, 118 aa, Artificial Sequence
  Synthetic
QVQLVQSGAEVKKPGSSVKVSCKASGYTFTSNYIHWVRQAPGQGLEWMGWIYPGDGDTNYNQKFQGRVTLTADKSTSTAYMELSSLRSEDTAVYYCAINYGGIWFAYWGQGTLVTVSS

SEQ ID NO: 45 - protein, 107 aa, Artificial Sequence
  Synthetic
DIQMTQSPSSLSASVGDRVTITCQASQDIGNKLIWYQQKPGKAPKLLIYYVTNLPGGVPSRFSGSGSGTDFTLTISSLQPEDFATYYCLQYKQNPLTFGQGTKLEIK

SEQ ID NO: 46 - protein, 107 aa, Artificial Sequence
  Synthetic
DIQMTQSPSSLSASVGDRVTITCQASQDIGNKLIWFQQKPGKAPKLLIHYVTNLPGGVPSRFSGSRSGSDYTLTISSLQPEDFATYYCLQYKQNPLTFGQGTKLEIK

SEQ ID NO: 47 - protein, 7 aa, Artificial Sequence
  Synthetic
TYDIGMG

SEQ ID NO: 48 - protein, 16 aa, Artificial Sequence
  Synthetic
HIWWNDREYYNSALQS

SEQ ID NO: 49 - protein, 13 aa, Artificial Sequence
  Synthetic
IDYFGSGQAWFTY

SEQ ID NO: 50 - protein, 12 aa, Artificial Sequence
  Synthetic
SSSSTISSTYLH

SEQ ID NO: 51 - protein, 7 aa, Artificial Sequence
  Synthetic
GTSNLAS

SEQ ID NO: 52 - protein, 9 aa, Artificial Sequence
  Synthetic
QQGSRIPFT

SEQ ID NO: 53 - protein, 16 aa, Artificial Sequence
  Synthetic
HIWWNDREYYSSALQS

SEQ ID NO: 54 - protein, 16 aa, Artificial Sequence
  Synthetic
HIWWNDREYYNPALQS

SEQ ID NO: 55 - protein, 123 aa, Artificial Sequence
  Synthetic
QLQLQESGPGLVKPSETLSLTCTVSGFSLNTYDIGMGWIRQPPGKGLEWIGHIWWNDREYYNSALQSRVTISVDTSKNQFSLKLSSVTAADTAVYYCARIDYFGSGQAWFTYWGQGTLVTVSS

SEQ ID NO: 56 - protein, 123 aa, Artificial Sequence
  Synthetic
QVQLQESGPGLVKPSETLSLTCTFSGFSLNTYDIGMGWIRQPPGKGLEWIGHIWWNDREYYNSALQSRVTISKDTSKTQVSLKLSSVTAADTAVYYCVRIDYFGSGQAWFTYWGQGTLVTVSS

SEQ ID NO: 57 - protein, 123 aa, Artificial Sequence
  Synthetic
QVQLQESGPGLVKPSETLSLTCTFSGFSLNTYDIGMGWIRQPPGKGLEWIAHIWWNDREYYNSALQSRVTISKDTSKTQVSLKLSSVTAADTAVYYCVRIDYFGSGQAWFTYWGQGTLVTVSS

SEQ ID NO: 58 - protein, 123 aa, Artificial Sequence
  Synthetic
QVQLQESGPGLVKPSETLSLTCTFSGFSLSTYDIGMGWIRQPPGKGLEWIAHIWWNDREYYNSALQSRVTISKDTSKTQVSLKLSSVTAADTAVYYCVRIDYFGSGQAWFTYWGQGTLVTVSS

SEQ ID NO: 59 - protein, 123 aa, Artificial Sequence
  Synthetic
QVQLQESGPGLVKPSETLSLTCTFSGFSLNTYDIGMGWIRQPPGKGLEWIAHIWWNDREYYSSALQSRVTISKDTSKTQVSLKLSSVTAADTAVYYCVRIDYFGSGQAWFTYWGQGTLVTVSS

SEQ ID NO: 60 - protein, 123 aa, Artificial Sequence
  Synthetic
QVQLQESGPGLVKPSETLSLTCTFSGFSLNTYDIGMGWIRQPPGKGLEWIAHIWWNDREYYNPALQSRVTISKDTSKTQVSLKLSSVTAADTAVYYCVRIDYFGSGQAWFTYWGQGTLVTVSS

SEQ ID NO: 61 - protein, 108 aa, Artificial Sequence
  Synthetic
EIVLTQSPGTLSLSPGERATLSCSSSSTISSTYLHWYQQKPGQAPRLLIYGTSNLASGIPDRFSGSGSGTDFTLTISRLEPEDFAVYYCQQGSRIPFTFGQGTKLEIK

SEQ ID NO: 62 - protein, 108 aa, Artificial Sequence
  Synthetic
EIVLTQSPGTLSLSPGERATLSCSSSSTISSTYLHWYQQKPGQAPRLLISGTSNLASGIPDRFSGSGSGTDYTLTISRLEPEDVAVYYCQQGSRIPFTFGQGTKLEIK

SEQ ID NO: 63 - protein, 5 aa, Artificial Sequence
  Synthetic
DYYMH

SEQ ID NO: 64 - protein, 17 aa, Artificial Sequence
  Synthetic
RIDPEDGETKYAPKFQG

SEQ ID NO: 65 - protein, 4 aa, Artificial Sequence
  Synthetic
SWAY

SEQ ID NO: 66 - protein, 12 aa, Artificial Sequence
  Synthetic
SASSSVSSSYLY

SEQ ID NO: 67 - protein, 7 aa, Artificial Sequence
  Synthetic
STSNLAS

SEQ ID NO: 68 - protein, 9 aa, Artificial Sequence
  Synthetic
HQWSSYPRT

SEQ ID NO: 69 - protein, 113 aa, Artificial Sequence
  Synthetic
EVQLVQSGAEVKKPGATVKISCKVSGFNIKDYYMHWVQQAPGKGLEWMGRIDPEDGETKYAPKFQGRVTITADTSTDTAYMELSSLRSEDTAVYYCATSWAYWGQGTTVTVSS

SEQ ID NO: 70 - protein, 113 aa, Artificial Sequence
  Synthetic
EVQLVQSGAEVKKPGATVKISCKASGFNIKDYYMHWVQQAPGKGLEWMGRIDPEDGETKYAPKFQGRVTITADTSTNTAYMELSSLRSEDTAVYYCSRSWAYWGQGTTVTVSS

SEQ ID NO: 71 - protein, 113 aa, Artificial Sequence
  Synthetic
QVQLVQSGAEVKKPGASVKVSCKASGFNIKDYYMHWVRQAPGQGLEWMGRIDPEDGETKYAPKFQGRVTMTRDTSTSTVYMELSSLRSEDTAVYYCARSWAYWGQGTTVTVSS

SEQ ID NO: 72 - protein, 113 aa, Artificial Sequence
  Synthetic
QVQLVQSGAEVKKPGASVKVSCKASGFNIKDYYMHWVRQAPGQGLEWMGRIDPEDGETKYAPKFQGRVTMTADTSTNTAYMELSSLRSEDTAVYYCSRSWAYWGQGTTVTVSS

SEQ ID NO: 73 - protein, 108 aa, Artificial Sequence
  Synthetic
EIVLTQSPATLSLSPGERATLSCSASSSVSSSYLYWYQQKPGQAPRLLIYSTSNLASGIPARFSGSGSGTDFTLTISSLEPEDFAVYYCHQWSSYPRTFGGGTKVEIK

SEQ ID NO: 74 - protein, 108 aa, Artificial Sequence
  Synthetic
EIVLTQSPATLSLSPGERATLSCSASSSVSSSYLYWYQQKPGQAPRLLIYSTSNLASGIPARFSGSGSGTDYTLTISSLEPEDAAVYFCHQWSSYPRTFGGGTKVEIK

SEQ ID NO: 75 - protein, 108 aa, Artificial Sequence
  Synthetic
EIVMTQSPPTLSLSPGERVTLSCSASSSVSSSYLYWYQQKPGQAPRLLIYSTSNLASGIPARFSGSGSGTDFTLTISSLQPEDFAVYYCHQWSSYPRTFGGGTKVEIK

SEQ ID NO: 76 - protein, 108 aa, Artificial Sequence
  Synthetic
EIVMTQSPPTLSLSPGERVTLSCSASSSVSSSYLYWYQQKPGQAPRLLIYSTSNLASGIPARFSGSGSGTDYTLTISSLQPEDAAVYFCHQWSSYPRTFGGGTKVEIK

SEQ ID NO: 77 - protein, 5 aa, Artificial Sequence
  Synthetic
DTYMH

SEQ ID NO: 78 - protein, 17 aa, Artificial Sequence
  Synthetic
RIDPANVNTIYDPKFQG

SEQ ID NO: 79 - protein, 11 aa, Artificial Sequence
  Synthetic
VGAYDGYDFDY

SEQ ID NO: 80 - protein, 15 aa, Artificial Sequence
  Synthetic
RASESVDNYGNSFMH

SEQ ID NO: 81 - protein, 7 aa, Artificial Sequence
  Synthetic
RASNLES

SEQ ID NO: 82 - protein, 9 aa, Artificial Sequence
  Synthetic
QQNNEDPLT

SEQ ID NO: 83 - protein, 120 aa, Artificial Sequence
  Synthetic
EVQLVQSGAEVKKPGATVKISCKVSGFNIKDTYMHWVQQAPGKGLEWMGRIDPANVNTIYDPKFQGRVTITADTSTDTAYMELSSLRSEDTAVYYCATVGAYDGYDFDYWGQGTTVTVSS

SEQ ID NO: 84 - protein, 120 aa, Artificial Sequence
  Synthetic
EVQLVQSGAEVKKPGATVKISCKASGFNIKDTYMHWVQQAPGKGLEWMGRIDPANVNTIYDPKFQGRVTITADTSTNTAYMELSSLRSEDTAVYYCARVGAYDGYDFDYWGQGTTVTVSS

SEQ ID NO: 85 - protein, 120 aa, Artificial Sequence
  Synthetic
QVQLVQSGAEVKKPGASVKVSCKASGFNIKDTYMHWVRQAPGQGLEWMGRIDPANVNTIYDPKFQGRVTMTRDTSTSTVYMELSSLRSEDTAVYYCARVGAYDGYDFDYWGQGTTVTVSS

SEQ ID NO: 86 - protein, 120 aa, Artificial Sequence
  Synthetic
QVQLVQSGAEVKKPGASVKVSCKASGFNIKDTYMHWVRQAPGQGLEWMGRIDPANVNTIYDPKFQGRVTMTADTSTNTAYMELSSLRSEDTAVYYCARVGAYDGYDFDYWGQGTTVTVSS

SEQ ID NO: 87 - protein, 111 aa, Artificial Sequence
  Synthetic
DIVMTQSPDSLAVSLGERATINCRASESVDNYGNSFMHWYQQKPGQPPKLLIYRASNLESGVPDRFSGSGSGTDFTLTISSLQAEDVAVYYCQQNNEDPLTFGQGTKLEIK

SEQ ID NO: 88 - protein, 111 aa, Artificial Sequence
  Synthetic
DIVLTQSPDSLAVSLGERATINCRASESVDNYGNSFMHWYQQKPGQPPKLLIYRASNLESGVPDRFSGSGSRTDFTLTISSLQAEDVAVYYCQQNNEDPLTFGQGTKLEIK

SEQ ID NO: 89 - protein, 111 aa, Artificial Sequence
  Synthetic
DIQMTQSPSSLSASVGDRVTITCRASESVDNYGNSFMHWYQQKPGKVPKLLIYRASNLESGVPSRFSGSGSGTDFTLTISSLQPEDVATYYCQQNNEDPLTFGQGTKLEIK

SEQ ID NO: 90 - protein, 111 aa, Artificial Sequence
  Synthetic
DIQLTQSPSSLSASVGDRVTITCRASESVDNYGNSFMHWYQQKPGKVPKLLIYRASNLESGVPSRFSGSGSRTDFTLTISSLQPEDVATYYCQQNNEDPLTFGQGTKLEIK

SEQ ID NO: 91 - protein, 5 aa, Artificial Sequence
  Synthetic
SYVMH

SEQ ID NO: 92 - protein, 17 aa, Artificial Sequence
  Synthetic
YINPYNDGTKYNEKFKG

SEQ ID NO: 93 - protein, 11 aa, Artificial Sequence
  Synthetic
SYYDYDGSFDY

SEQ ID NO: 94 - protein, 11 aa, Artificial Sequence
  Synthetic
KASQDVTTAVA

SEQ ID NO: 95 - protein, 7 aa, Artificial Sequence
  Synthetic
SASYRYT

SEQ ID NO: 96 - protein, 9 aa, Artificial Sequence
  Synthetic
QQHYSTPWT

SEQ ID NO: 97 - protein, 120 aa, Artificial Sequence
  Synthetic
QVQLVQSGAEVKKPGASVKVSCKASGYTFTSYVMHWVRQAPGQRLEWMGYINPYNDGTKYNEKFKGRVTITRDTSASTAYMELSSLRSEDTAVYYCARSYYDYDGSFDYWGQGTTVTVSS

SEQ ID NO: 98 - protein, 120 aa, Artificial Sequence
  Synthetic
QVQLVQSGAEVKKPGASVKVSCKASGYTFTSYVMHWVRQAPGQRLEWMGYINPYNDGTKYNEKFKGRVTITSDKSASTAYMELSSLRSEDTAVYYCARSYYDYDGSFDYWGQGTTVTVSS

SEQ ID NO: 99 - protein, 120 aa, Artificial Sequence
  Synthetic
QVQLVQSGAEVKKPGASVKVSCKASGYTFTSYVMHWVRQAPGQGLEWMGYINPYNDGTKYNEKFKGRVTMTRDTSTSTVYMELSSLRSEDTAVYYCARSYYDYDGSFDYWGQGTTVTVSS

SEQ ID NO: 100 - protein, 120 aa, Artificial Sequence
  Synthetic
QVQLVQSGAEVKKPGASVKVSCKASGYTFTSYVMHWVRQAPGQGLEWMGYINPYNDGTKYNEKFKGRVTMTSDKSTSTAYMELSSLRSEDTAVYYCARSYYDYDGSFDYWGQGTTVTVSS

SEQ ID NO: 101 - protein, 107 aa, Artificial Sequence
  Synthetic
DIQMTQSPSSLSASVGDRVTITCKASQDVTTAVAWYQQKPGKAPKLLIYSASYRYTGVPSRFSGSGSGTDFTFTISSLQPEDIATYYCQQHYSTPWTFGGGTKVEIK

SEQ ID NO: 102 - protein, 107 aa, Artificial Sequence
  Synthetic
DIQMTQSPSSLSASVGDRVTITCKASQDVTTAVAWYQQKPGKVPKLLIYSASYRYTGVPSRFSGSGSGTDFTLTISSLQPEDVATYYCQQHYSTPWTFGGGTKVEIK

SEQ ID NO: 103 - protein, 5 aa, Artificial Sequence
  Synthetic
DTYIH

SEQ ID NO: 104 - protein, 17 aa, Artificial Sequence
  Synthetic
RIDPADGDTKHNPKFHD

SEQ ID NO: 105 - protein, 5 aa, Artificial Sequence
  Synthetic
GNYVN

SEQ ID NO: 106 - protein, 12 aa, Artificial Sequence
  Synthetic
RASSSVTSSYLY

SEQ ID NO: 107 - protein, 7 aa, Artificial Sequence
  Synthetic
SASNLAS

SEQ ID NO: 108 - protein, 9 aa, Artificial Sequence
  Synthetic
HQWYSYPRT

SEQ ID NO: 109 - protein, 114 aa, Artificial Sequence
  Synthetic
EVQLVQSGAEVKKPGATVKISCKVSGFNFEDTYIHWVQQAPGKGLEWMGRIDPADGDTKHNPKFHDRVTITADTSTDTAYMELSSLRSEDTAVYYCATGNYVNWGQGTTVTVSS

SEQ ID NO: 110 - protein, 114 aa, Artificial Sequence
  Synthetic
EVQLVQSGAEVKKPGATVKISCKASGFNFEDTYIHWVQQAPGKGLEWMGRIDPADGDTKHNPKFHDRVTITVDTSTNTAYMELSSLRSEDTAVYYCVRGNYVNWGQGTTVTVSS

SEQ ID NO: 111 - protein, 114 aa, Artificial Sequence
  Synthetic
QVQLVQSGAEVKKPGASVKVSCKASGFNFEDTYIHWVRQAPGQGLEWMGRIDPADGDTKHNPKFHDRVTMTRDTSTSTVYMELSSLRSEDTAVYYCARGNYVNWGQGTTVTVSS

SEQ ID NO: 112 - protein, 114 aa, Artificial Sequence
  Synthetic
QVQLVQSGAEVKKPGASVKVSCKASGFNFEDTYIHWVRQAPGQGLEWMGRIDPADGDTKHNPKFHDRVTMTVDTSTNTAYMELSSLRSEDTAVYYCVRGNYVNWGQGTTVTVSS

SEQ ID NO: 113 - protein, 108 aa, Artificial Sequence
  Synthetic
EIVLTQSPATLSLSPGERATLSCRASSSVTSSYLYWYQQKPGQAPRLLIYSASNLASGIPARFSGSGSGTDFTLTISSLEPEDFAVYYCHQWYSYPRTFGGGTKVEIK

SEQ ID NO: 114 - protein, 108 aa, Artificial Sequence
  Synthetic
EIVLTQSPATLSLSPGERATLSCRASSSVTSSYLYWYQQKPGQAPRLLIYSASNLASGIPARFSGSGSGTDYTLTISSLEPEDAAVYFCHQWYSYPRTFGGGTKVEIK

SEQ ID NO: 115 - protein, 108 aa, Artificial Sequence
  Synthetic
EIVLTQSPGTLSLSPGERATLSCRASSSVTSSYLYWYQQKPGQAPRLLIYSASNLASGIPDRFSGSGSGTDFTLTISRLEPEDFAVYYCHQWYSYPRTFGGGTKVEIK

SEQ ID NO: 116 - protein, 108 aa, Artificial Sequence
  Synthetic
EIVLTQSPGTLSLSPGERATLSCRASSSVTSSYLYWYQQKPGQAPRLLIYSASNLASGIPDRFSGSGSGTDYTLTISRLEPEDAAVYFCHQWYSYPRTFGGGTKVEIK

SEQ ID NO: 117 - protein, 108 aa, Artificial Sequence
  Synthetic
EIVMTQSPPTLSLSPGERVTLSCRASSSVTSSYLYWYQQKPGQAPRLLIYSASNLASGIPARFSGSGSGTDFTLTISSLQPEDFAVYYCHQWYSYPRTFGGGTKVEIK

SEQ ID NO: 118 - protein, 108 aa, Artificial Sequence
  Synthetic
EIVMTQSPPTLSLSPGERVTLSCRASSSVTSSYLYWYQQKPGQAPRLLIYSASNLASGIPARFSGSGSGTDYTLTISSLQPEDAAVYFCHQWYSYPRTFGGGTKVEIK

SEQ ID NO: 119 - protein, 25 aa, Artificial Sequence
  Synthetic
DVQLVESGGGLVQAGDSLRLSCAAS

SEQ ID NO: 120 - protein, 25 aa, Artificial Sequence
  Synthetic
EVQLVESGGRLVRAGDSLRLSCAAS

SEQ ID NO: 121 - protein, 25 aa, Artificial Sequence
  Synthetic
QVQLVESGGGLVQAGGSLRLACSAS

SEQ ID NO: 122 - protein, 25 aa, Artificial Sequence
  Synthetic
QVQLVESGGGLVQAGGSLRLSCAAS

SEQ ID NO: 123 - protein, 25 aa, Artificial Sequence
  Synthetic
AVQLVESGGGLVQAGGSLRLSCAAS

SEQ ID NO: 124 - protein, 25 aa, Artificial Sequence
  Synthetic
AVQLVESGGGLVQAGDSLRLSCTAS

SEQ ID NO: 125 - protein, 25 aa, Artificial Sequence
  Synthetic
AVQLVESGGGLVQAGDSLRLACAAS

SEQ ID NO: 126 - protein, 25 aa, Artificial Sequence
  Synthetic
EVQLVESGGGLVRAGGSLRLSCAAS

SEQ ID NO: 127 - protein, 25 aa, Artificial Sequence
  Synthetic
EVQLVESGGGLVQAGDSLRLSCTAS

SEQ ID NO: 128 - protein, 25 aa, Artificial Sequence
  Synthetic
QVQLVESGGGLVQAGGSLRLSCAAS

SEQ ID NO: 129 - protein, 25 aa, Artificial Sequence
  Synthetic
AVQLVESGGGLVQAGDSLRLSCTAS

SEQ ID NO: 130 - protein, 25 aa, Artificial Sequence
  Synthetic
EVQLVESGGGLVQAGDSLRLSCAAS

SEQ ID NO: 131 - protein, 25 aa, Artificial Sequence
  Synthetic
QVQLVESGGGLVQAGGSLRLSCAVS

SEQ ID NO: 132 - protein, 25 aa, Artificial Sequence
  Synthetic
EVQLVESGGGLVQAGGSLSLSCAVS

SEQ ID NO: 133 - protein, 25 aa, Artificial Sequence
  Synthetic
QVQLVESGGGLVQAGDSLRLSCAAS

SEQ ID NO: 134 - protein, 25 aa, Artificial Sequence
  Synthetic
QVQLVESGGGLVQAGGSLKLSCAAS

SEQ ID NO: 135 - protein, 25 aa, Artificial Sequence
  Synthetic
QVQLVESGGGLVQAGDSLRLSCAAS

SEQ ID NO: 136 - protein, 25 aa, Artificial Sequence
  Synthetic
QVQLVESGGGLVQAGGSLRLSCAAS

SEQ ID NO: 137 - protein, 25 aa, Artificial Sequence
  Synthetic
QVKLEESGGGLVQAGGSLRLSCVAS

SEQ ID NO: 138 - protein, 25 aa, Artificial Sequence
  Synthetic
AVQLVESGGGLVQAGDSLRLSCAAS

SEQ ID NO: 139 - protein, 25 aa, Artificial Sequence
  Synthetic
DVQLVESGGGLVQAGDSLRLTCSAS

SEQ ID NO: 140 - protein, 25 aa, Artificial Sequence
  Synthetic
EVQLVESGGGLVQAGDSLRLSCVAS

SEQ ID NO: 141 - protein, 25 aa, Artificial Sequence
  Synthetic
EVQLVESGGGLVQAGDSLRLSCAAS

SEQ ID NO: 142 - protein, 25 aa, Artificial Sequence
  Synthetic
QVKLEESGGGLVQVGDSLRLSCAAS

SEQ ID NO: 143 - protein, 25 aa, Artificial Sequence
  Synthetic
QVKLEESGGGLVQVGDSLRLSCAAS

SEQ ID NO: 144 - protein, 25 aa, Artificial Sequence
  Synthetic
QVQLVESGGGLVQAGGSLRLSCAAS

SEQ ID NO: 145 - protein, 25 aa, Artificial Sequence
  Synthetic
QVQLVESGGGLVQAGGSLRLSCAAS

SEQ ID NO: 146 - protein, 25 aa, Artificial Sequence [sequence omitted]

SEQ ID NO: 147 - protein, 25 aa, Artificial Sequence [sequence omitted]

SEQ ID NO: 148 - protein, 25 aa, Artificial Sequence [sequence omitted]

SEQ ID NO: 149 - protein, 25 aa, Artificial Sequence [sequence omitted]

SEQ ID NO: 150 - protein, 25 aa, Artificial Sequence [sequence omitted]

SEQ ID NO: 151 - protein, 25 aa, Artificial Sequence [sequence omitted]

SEQ ID NO: 152 - protein, 25 aa, Artificial Sequence [sequence omitted]

SEQ ID NO: 153 - protein, 25 aa, Artificial Sequence [sequence omitted]

SEQ ID NO: 154 - protein, 25 aa, Artificial Sequence [sequence omitted]

SEQ ID NO: 155 - protein, 25 aa, Artificial Sequence
  Synthetic
EVQLVESGGGLVQPGGSLRLSCTAS

SEQ ID NO: 156 - protein, 25 aa, Artificial Sequence [sequence omitted]

SEQ ID NO: 157 - protein, 25 aa, Artificial Sequence
  Synthetic
EVQLVESGGGLVQAGDSLRLSCVAS

SEQ ID NO: 158 - protein, 25 aa, Artificial Sequence
  Synthetic
EVQLVESGGGLVQAGDSLRLSCVAS

SEQ ID NO: 159 - protein, 25 aa, Artificial Sequence
  Synthetic
EVQLVESGGGLVQAGDSLRLSCVAS

SEQ ID NO: 160 - protein, 25 aa, Artificial Sequence [sequence omitted]

SEQ ID NO: 161 - protein, 25 aa, Artificial Sequence [sequence omitted]

SEQ ID NO: 162 - protein, 25 aa, Artificial Sequence
  Synthetic
EVQLVESGGGLVQPGGSLRLSCVAS

SEQ ID NO: 163 - protein, 25 aa, Artificial Sequence [sequence omitted]

SEQ ID NO: 164 - protein, 25 aa, Artificial Sequence [sequence omitted]

SEQ ID NO: 165 - protein, 25 aa, Artificial Sequence [sequence omitted]

SEQ ID NO: 166 - protein, 25 aa, Artificial Sequence [sequence omitted]

SEQ ID NO: 167 - protein, 25 aa, Artificial Sequence [sequence omitted]

SEQ ID NO: 168 - protein, 25 aa, Artificial Sequence [sequence omitted]

SEQ ID NO: 169 - protein, 10 aa, Artificial Sequence
  Synthetic
GRTFISYAVG

SEQ ID NO: 170 - protein, 10 aa, Artificial Sequence
  Synthetic
GRTFLSYAVG

SEQ ID NO: 171 - protein, 10 aa, Artificial Sequence
  Synthetic
GRTFLTYALG

SEQ ID NO: 172 - protein, 10 aa, Artificial Sequence
  Synthetic
GRTFITYAIG

SEQ ID NO: 173 - protein, 10 aa, Artificial Sequence
  Synthetic
GRTFITYAIG

SEQ ID NO: 174 - protein, 10 aa, Artificial Sequence
  Synthetic
GRTFLSYAVG

SEQ ID NO: 175 - protein, 10 aa, Artificial Sequence
  Synthetic
GRTFLTYALG

SEQ ID NO: 176 - protein, 10 aa, Artificial Sequence
  Synthetic
GRTFITYAIG

SEQ ID NO: 177 - protein, 10 aa, Artificial Sequence
  Synthetic
GRTFLSYAVG

SEQ ID NO: 178 - protein, 10 aa, Artificial Sequence
  Synthetic
GRTFITYAIG

SEQ ID NO: 179 - protein, 10 aa, Artificial Sequence
  Synthetic
GRTFLTYAVG

SEQ ID NO: 180 - protein, 10 aa, Artificial Sequence
  Synthetic
GRTFLTYAVG

SEQ ID NO: 181 - protein, 10 aa, Artificial Sequence
  Synthetic
GRPVSSAVMG

SEQ ID NO: 182 - protein, 10 aa, Artificial Sequence
  Synthetic
GRTLTGLLIG

SEQ ID NO: 183 - protein, 10 aa, Artificial Sequence
  Synthetic
GRTFLTLAVG

SEQ ID NO: 184 - protein, 10 aa, Artificial Sequence
  Synthetic
GRTFITYAMG

SEQ ID NO: 185 - protein, 10 aa, Artificial Sequence
  Synthetic
GRTFLTLAVG

SEQ ID NO: 186 - protein, 10 aa, Artificial Sequence
  Synthetic
GRTFITYAIG

SEQ ID NO: 187 - protein, 10 aa, Artificial Sequence
  Synthetic
GRTFSRYAMG

SEQ ID NO: 188 - protein, 10 aa, Artificial Sequence
  Synthetic
GRTFLSYAVG

SEQ ID NO: 189 - protein, 10 aa, Artificial Sequence
  Synthetic
GRTFISYAVG

SEQ ID NO: 190 - protein, 10 aa, Artificial Sequence
  Synthetic
GRTFVTYGMG

SEQ ID NO: 191 - protein, 10 aa, Artificial Sequence
  Synthetic
GRTFLSYAVG

SEQ ID NO: 192 - protein, 10 aa, Artificial Sequence
  Synthetic
VSSFSINDMG

SEQ ID NO: 193 - protein, 10 aa, Artificial Sequence
  Synthetic
GRTFSNYTMA

SEQ ID NO: 194 - protein, 10 aa, Artificial Sequence
  Synthetic
GRTFITYAIG

SEQ ID NO: 195 - protein, 10 aa, Artificial Sequence
  Synthetic
GRTFITYAIG

SEQ ID NO: 196 - protein, 10 aa, Artificial Sequence
  Synthetic
GRTFITYAIG

SEQ ID NO: 197 - protein, 10 aa, Artificial Sequence
  Synthetic
GRTFITYAIG

SEQ ID NO: 198 - protein, 10 aa, Artificial Sequence
  Synthetic
GRTFITYAIG

SEQ ID NO: 199 - protein, 10 aa, Artificial Sequence
  Synthetic
GRTFITYAIG

SEQ ID NO: 200 - protein, 10 aa, Artificial Sequence
  Synthetic
GRTFITYAIG

SEQ ID NO: 201 - protein, 10 aa, Artificial Sequence
  Synthetic
GRTFITYAIG

SEQ ID NO: 202 - protein, 10 aa, Artificial Sequence
  Synthetic
GRTFLTYAVG

SEQ ID NO: 203 - protein, 10 aa, Artificial Sequence
  Synthetic
GRTFLTYAVG

SEQ ID NO: 204 - protein, 10 aa, Artificial Sequence
  Synthetic
GRTFLTYAVG

SEQ ID NO: 205 - protein, 10 aa, Artificial Sequence
  Synthetic
GRTFLTYAVG

SEQ ID NO: 206 - protein, 10 aa, Artificial Sequence
  Synthetic
GRTFLTYAVG

SEQ ID NO: 207 - protein, 10 aa, Artificial Sequence
  Synthetic
GRTFVTYGMG

SEQ ID NO: 208 - protein, 10 aa, Artificial Sequence
  Synthetic
GRTFVTYGMG

SEQ ID NO: 209 - protein, 10 aa, Artificial Sequence
  Synthetic
GRTFVTYGMG

SEQ ID NO: 210 - protein, 10 aa, Artificial Sequence
  Synthetic
GRTFVTYGMG

SEQ ID NO: 211 - protein, 10 aa, Artificial Sequence
  Synthetic
GRTFVTYGMG

SEQ ID NO: 212 - protein, 10 aa, Artificial Sequence
  Synthetic
GRTFVTYGMG

SEQ ID NO: 213 - protein, 10 aa, Artificial Sequence
  Synthetic
GRTFVTYGMG

SEQ ID NO: 214 - protein, 10 aa, Artificial Sequence
  Synthetic
GRTFVTYGMG

SEQ ID NO: 215 - protein, 10 aa, Artificial Sequence
  Synthetic
GRTFVTYGMG

SEQ ID NO: 216 - protein, 10 aa, Artificial Sequence
  Synthetic
GRTFISYAVG

SEQ ID NO: 217 - protein, 10 aa, Artificial Sequence
  Synthetic
GRTFLTLAVG

SEQ ID NO: 218 - protein, 10 aa, Artificial Sequence
  Synthetic
GRTFLTYAVG

SEQ ID NO: 219 - protein, 14 aa, Artificial Sequence
  Synthetic
WFRQAPGSEREFVA

SEQ ID NO: 220 - protein, 14 aa, Artificial Sequence
  Synthetic
WFRQAPGTEREFVA

SEQ ID NO: 221 - protein, 14 aa, Artificial Sequence
  Synthetic
WFRQAPGKEREFVA

SEQ ID NO: 222 - protein, 14 aa, Artificial Sequence
  Synthetic
WFRQAPGKEREFVT

SEQ ID NO: 223 - protein, 14 aa, Artificial Sequence
  Synthetic
WFRQAPGKEREFVS

SEQ ID NO: 224 - protein, 14 aa, Artificial Sequence
  Synthetic
WFRQAPGTEREFVA

SEQ ID NO: 225 - protein, 14 aa, Artificial Sequence
  Synthetic
WFRQAPGKEREFVA

SEQ ID NO: 226 - protein, 14 aa, Artificial Sequence
  Synthetic
WFRQAPGKEREFVT

SEQ ID NO: 227 - protein, 14 aa, Artificial Sequence
  Synthetic
WFRQAPGTEREFVA

SEQ ID NO: 228 - protein, 14 aa, Artificial Sequence
  Synthetic
WFRQAPGKEREFVS

SEQ ID NO: 229 - protein, 14 aa, Artificial Sequence
  Synthetic
WFRQAPGTEREFVA

SEQ ID NO: 230 - protein, 14 aa, Artificial Sequence
  Synthetic
WFRQAPGTEREFVA

SEQ ID NO: 231 - protein, 14 aa, Artificial Sequence
  Synthetic
WFRQAPGKEREFVG

SEQ ID NO: 232 - protein, 14 aa, Artificial Sequence
  Synthetic
WFRQAPGKERELVA

SEQ ID NO: 233 - protein, 14 aa, Artificial Sequence
  Synthetic
WFRQAPGTEREFVA

SEQ ID NO: 234 - protein, 14 aa, Artificial Sequence
  Synthetic
WFRQAPGKERELVA

SEQ ID NO: 235 - protein, 14 aa, Artificial Sequence
  Synthetic
WFRQAPGTEREFVA

SEQ ID NO: 236 - protein, 14 aa, Artificial Sequence
  Synthetic
WFRQAPGKEREFVT

SEQ ID NO: 237 - protein, 14 aa, Artificial Sequence
  Synthetic
WFRQAPGKEREFVS

SEQ ID NO: 238 - protein, 14 aa, Artificial Sequence
  Synthetic
WFRQAPGTEREFVA

SEQ ID NO: 239 - protein, 14 aa, Artificial Sequence
  Synthetic
WFRQAPGTEREFVA

SEQ ID NO: 240 - protein, 14 aa, Artificial Sequence
  Synthetic
WFRQAPGKEREFVA

SEQ ID NO: 241 - protein, 14 aa, Artificial Sequence
  Synthetic
WFRQAPGTEREFVA

SEQ ID NO: 242 - protein, 14 aa, Artificial Sequence
  Synthetic
WYRQAPGKQRELVA

SEQ ID NO: 243 - protein, 14 aa, Artificial Sequence
  Synthetic
WFRQFPGKEREFVA

SEQ ID NO: 244 - protein, 14 aa, Artificial Sequence
  Synthetic
WFRQAPGKEREFVS

SEQ ID NO: 245 - protein, 14 aa, Artificial Sequence
  Synthetic
WFRQAPGKEREFVS

SEQ ID NO: 246 - protein, 14 aa, Artificial Sequence
  Synthetic
WVRQAPGKGLEWVS

SEQ ID NO: 247 - protein, 14 aa, Artificial Sequence
  Synthetic
WFRQAPGKGLEWVS

SEQ ID NO: 248 - protein, 14 aa, Artificial Sequence
  Synthetic
WFRQAPGKGLEFVS

SEQ ID NO: 249 - protein, 14 aa, Artificial Sequence
  Synthetic
WFRQAPGKGREFVS

SEQ ID NO: 250 - protein, 14 aa, Artificial Sequence
  Synthetic
WFRQAPGKGREFVS

SEQ ID NO: 251 - protein, 14 aa, Artificial Sequence
  Synthetic
WFRQAPGKGREFVS

SEQ ID NO: 252 - protein, 14 aa, Artificial Sequence
  Synthetic
WVRQAPGKGLEWVS

SEQ ID NO: 253 - protein, 14 aa, Artificial Sequence
  Synthetic
WFRQAPGKGLEWVA

SEQ ID NO: 254 - protein, 14 aa, Artificial Sequence
  Synthetic
WFRQAPGKGLEFVA

SEQ ID NO: 255 - protein, 14 aa, Artificial Sequence
  Synthetic
WFRQAPGKGLEFVA

SEQ ID NO: 256 - protein, 14 aa, Artificial Sequence
  Synthetic
WFRQAPGKGREFVS

SEQ ID NO: 257 - protein, 14 aa, Artificial Sequence
  Synthetic
WFRQAPGKEREFVA

SEQ ID NO: 258 - protein, 14 aa, Artificial Sequence
  Synthetic
WFRQAPGKEREFVA

SEQ ID NO: 259 - protein, 14 aa, Artificial Sequence
  Synthetic
WFRQAPGKEREFVA

SEQ ID NO: 260 - protein, 14 aa, Artificial Sequence
  Synthetic
WVRQAPGKGLEWVS

SEQ ID NO: 261 - protein, 14 aa, Artificial Sequence
  Synthetic
WFRQAPGKGLEFVA

SEQ ID NO: 262 - protein, 14 aa, Artificial Sequence
  Synthetic
WFRQAPGKGREFVS

SEQ ID NO: 263 - protein, 14 aa, Artificial Sequence
  Synthetic
WFRQAPGKGREFVS

SEQ ID NO: 264 - protein, 14 aa, Artificial Sequence
  Synthetic
WFRQAPGKGREFVS

SEQ ID NO: 265 - protein, 14 aa, Artificial Sequence
  Synthetic
WFRQAPGKGREFVS

SEQ ID NO: 266 - protein, 14 aa, Artificial Sequence
  Synthetic
WFRQAPGKGREFVS

SEQ ID NO: 267 - protein, 14 aa, Artificial Sequence
  Synthetic
WFRQAPGKGREFVS

SEQ ID NO: 268 - protein, 14 aa, Artificial Sequence
  Synthetic
WFRQAPGKGLEFVS

SEQ ID NO: 269 - protein, 17 aa, Artificial Sequence
  Synthetic
GIRWNGIHTDYADSVKG

SEQ ID NO: 270 - protein, 17 aa, Artificial Sequence
  Synthetic
GIRWSGGYTDYAEAVKG

SEQ ID NO: 271 - protein, 17 aa, Artificial Sequence
  Synthetic
GVSWSGSGTKYADSVKG

SEQ ID NO: 272 - protein, 17 aa, Artificial Sequence
  Synthetic
AINWSGSMTSYADSVKG

SEQ ID NO: 273 - protein, 17 aa, Artificial Sequence
  Synthetic
AINWSGSMTSYADSVKG

SEQ ID NO: 274 - protein, 17 aa, Artificial Sequence
  Synthetic
GIRWSGEHTDYAASVKG

SEQ ID NO: 275 - protein, 17 aa, Artificial Sequence
  Synthetic
GVSWSGSSTKYADSVKG

SEQ ID NO: 276 - protein, 17 aa, Artificial Sequence
  Synthetic
AINWSGSMTSYADSVKG

SEQ ID NO: 277 - protein, 17 aa, Artificial Sequence
  Synthetic
GIRWSGGSTDYSDSVKG

SEQ ID NO: 278 - protein, 17 aa, Artificial Sequence
  Synthetic
AINWSGSMTSYADSVKG

SEQ ID NO: 279 - protein, 17 aa, Artificial Sequence
  Synthetic
GIRWSGGYTDYADSVKG

SEQ ID NO: 280 - protein, 17 aa, Artificial Sequence
  Synthetic
GIRWSGESTDYAESVKG

SEQ ID NO: 281 - protein, 17 aa, Artificial Sequence
  Synthetic
RLTSSATSTFYAESVKG

SEQ ID NO: 282 - protein, 17 aa, Artificial Sequence
  Synthetic
IISWTYGSTNYADSVKG

SEQ ID NO: 283 - protein, 17 aa, Artificial Sequence
  Synthetic
GIRWSGSGTDYADSVKG

SEQ ID NO: 284 - protein, 17 aa, Artificial Sequence
  Synthetic
AISWSGSSTYSADSVKG

SEQ ID NO: 285 - protein, 17 aa, Artificial Sequence
  Synthetic
GIRWSGSGTDYADSVKG

SEQ ID NO: 286 - protein, 17 aa, Artificial Sequence
  Synthetic
AINWSGSMTSYADSVKG

SEQ ID NO: 287 - protein, 17 aa, Artificial Sequence
  Synthetic
TSTGSGGLTSYANSVKG

SEQ ID NO: 288 - protein, 17 aa, Artificial Sequence
  Synthetic
GIRWSGLHTDYADSVKG

SEQ ID NO: 289 - protein, 17 aa, Artificial Sequence
  Synthetic
GIRWNGISTDYTDSVKG

SEQ ID NO: 290 - protein, 17 aa, Artificial Sequence
  Synthetic
AINWSGSMTSYGDSVKG

SEQ ID NO: 291 - protein, 17 aa, Artificial Sequence
  Synthetic
GIRWSGGSTDYADSVKG

SEQ ID NO: 292 - protein, 16 aa, Artificial Sequence
  Synthetic
TIASGGSTNYADSVKG

SEQ ID NO: 293 - protein, 17 aa, Artificial Sequence
  Synthetic
VVSRGGGATDYADSVKG

SEQ ID NO: 294 - protein, 17 aa, Artificial Sequence
  Synthetic
AISWSGSMTSYADSVKG

SEQ ID NO: 295 - protein, 17 aa, Artificial Sequence
  Synthetic
AIQWSGSMTSYADSVKG

SEQ ID NO: 296 - protein, 17 aa, Artificial Sequence
  Synthetic
AIQWSGSMTSYADSVKG

SEQ ID NO: 297 - protein, 17 aa, Artificial Sequence
  Synthetic
AIQWSGSMTSYADSVKG

SEQ ID NO: 298 - protein, 17 aa, Artificial Sequence
  Synthetic
AIQWSGSMTSYADSVKG

SEQ ID NO: 299 - protein, 17 aa, Artificial Sequence
  Synthetic
AISWSGSMTSYADSVKG

SEQ ID NO: 300 - protein, 17 aa, Artificial Sequence
  Synthetic
AISWSGSITSYADSVKG

SEQ ID NO: 301 - protein, 17 aa, Artificial Sequence
  Synthetic
AISWSGSLTSYADSVKG

SEQ ID NO: 302 - protein, 17 aa, Artificial Sequence
  Synthetic
GIRWSGGYTDYADSVKG

SEQ ID NO: 303 - protein, 17 aa, Artificial Sequence
  Synthetic
GIRWSGGYTDYADSVKG

SEQ ID NO: 304 - protein, 17 aa, Artificial Sequence
  Synthetic
GIRWSGGYTDYADSVKG

SEQ ID NO: 305 - protein, 17 aa, Artificial Sequence
  Synthetic
GIRWSGGYTDYADSVKG

SEQ ID NO: 306 - protein, 17 aa, Artificial Sequence
  Synthetic
GIRWSGGYTDYADSVKG

SEQ ID NO: 307 - protein, 17 aa, Artificial Sequence
  Synthetic
AIAWSGSMTSYGDSVKG

SEQ ID NO: 308 - protein, 17 aa, Artificial Sequence
  Synthetic
AISWSGSMTSYGDSVKG

SEQ ID NO: 309 - protein, 17 aa, Artificial Sequence
  Synthetic
AIQWSGSMTSYGDSVKG

SEQ ID NO: 310 - protein, 17 aa, Artificial Sequence
  Synthetic
AIQWSGSMTSYGDSVKG

SEQ ID NO: 311 - protein, 17 aa, Artificial Sequence
  Synthetic
AIQWSGSMTSYGDSVKG

SEQ ID NO: 312 - protein, 17 aa, Artificial Sequence
  Synthetic
AIQWSGSMTSYGDSVKG

SEQ ID NO: 313 - protein, 17 aa, Artificial Sequence
  Synthetic
AISWSGSMTSYGDSVKG

SEQ ID NO: 314 - protein, 17 aa, Artificial Sequence
  Synthetic
AISWSGSITSYGDSVKG

SEQ ID NO: 315 - protein, 17 aa, Artificial Sequence
  Synthetic
AISWSGSLTSYGDSVKG

SEQ ID NO: 316 - protein, 17 aa, Artificial Sequence
  Synthetic
GIRWSGIHTDYADSVKG

SEQ ID NO: 317 - protein, 17 aa, Artificial Sequence
  Synthetic
GIRWSGSGTDYADSVKG

SEQ ID NO: 318 - protein, 17 aa, Artificial Sequence
  Synthetic
GIRWSGGYTDYADSVKG

SEQ ID NO: 319 - protein, 32 aa, Artificial Sequence
  Synthetic
RFTISRDNAKNTVTLEMTSLKPEDTAVYYCAA

SEQ ID NO: 320 - protein, 32 aa, Artificial Sequence
  Synthetic
RFTISRDNAKNTVYLQMNSLKPEDTAVYYCAA

SEQ ID NO: 321 - protein, 32 aa, Artificial Sequence
  Synthetic
RFTISRDNAKNTVYMQMNSLKPEDTAVYYCAA

SEQ ID NO: 322 - protein, 32 aa, Artificial Sequence
  Synthetic
RFTISRDNNKNMVYLQMNSLKPEDTAVYYCAA

SEQ ID NO: 323 - protein, 32 aa, Artificial Sequence
  Synthetic
RFTISRDNAKNTVYLQMNGLKPEDTAVYYCAA

SEQ ID NO: 324 - protein, 32 aa, Artificial Sequence; identical to SEQ ID NO: 320
  Synthetic

SEQ ID NO: 325 - protein, 32 aa, Artificial Sequence; identical to SEQ ID NO: 321
  Synthetic

SEQ ID NO: 326 - protein, 32 aa, Artificial Sequence
  Synthetic
RFTISRDNNKNTVYLQMNSLKPEDTALYYCAA

SEQ ID NO: 327 - protein, 32 aa, Artificial Sequence; identical to SEQ ID NO: 320
  Synthetic

SEQ ID NO: 328 - protein, 32 aa, Artificial Sequence; identical to SEQ ID NO: 323
  Synthetic

SEQ ID NO: 329 - protein, 32 aa, Artificial Sequence; identical to SEQ ID NO: 320
  Synthetic

SEQ ID NO: 330 - protein, 32 aa, Artificial Sequence
  Synthetic
RFTISRDDTKNTVYLQMNSLKPEDTAVYYCAA

SEQ ID NO: 331 - protein, 32 aa, Artificial Sequence
  Synthetic
RFTISRDNAKNTVYLQMNNLKPEDTAVYYCAA

SEQ ID NO: 332 - protein, 32 aa, Artificial Sequence
  Synthetic
RFTISRDNAKNTVLLQMNSLKPEDTAVYYCSA

SEQ ID NO: 333 - protein, 32 aa, Artificial Sequence; identical to SEQ ID NO: 320
  Synthetic

SEQ ID NO: 334 - protein, 32 aa, Artificial Sequence; identical to SEQ ID NO: 320
  Synthetic

SEQ ID NO: 335 - protein, 32 aa, Artificial Sequence
  Synthetic
RFTISRDNAANTVYLQMNSLKPEDTAVYYCAA

SEQ ID NO: 336 - protein, 32 aa, Artificial Sequence
  Synthetic
RFTISRDNAKNTVYLQINGLKSEDTAVYYCAA

SEQ ID NO: 337 - protein, 32 aa, Artificial Sequence
  Synthetic
RFTISRDNAKNTVYLQMNNLKPEDTAIYYCAA

SEQ ID NO: 338 - protein, 32 aa, Artificial Sequence
  Synthetic
RFTISRDNAKNTVYLQMNSLKPEDTAIYYCAA

SEQ ID NO: 339 - protein, 32 aa, Artificial Sequence; identical to SEQ ID NO: 338
  Synthetic

SEQ ID NO: 340 - protein, 32 aa, Artificial Sequence
  Synthetic
RFAISRDNAKNTVYLQMNSLKPEDTAVYYCAA

SEQ ID NO: 341 - protein, 32 aa, Artificial Sequence; identical to SEQ ID NO: 320
  Synthetic

SEQ ID NO: 342 - protein, 32 aa, Artificial Sequence
  Synthetic
RFTISRDNVKNTVYLQMNGLKPEDTAVYYCNA

SEQ ID NO: 343 - protein, 32 aa, Artificial Sequence
  Synthetic
RFTISRDNAKNTMYLQMNSLKTEDTAVYYCAA

SEQ ID NO: 344 - protein, 32 aa, Artificial Sequence; identical to SEQ ID NO: 323
  Synthetic

SEQ ID NO: 345 - protein, 32 aa, Artificial Sequence; identical to SEQ ID NO: 323
  Synthetic

SEQ ID NO: 346 - protein, 32 aa, Artificial Sequence
  Synthetic
RFTISRDNSKNTLYLQMNSLRAEDTAVYYCAK

SEQ ID NO: 347 - protein, 32 aa, Artificial Sequence
  Synthetic
RFTISRDNSKNTVYLQMNSLRAEDTAVYYCAA

SEQ ID NO: 348 - protein, 32 aa, Artificial Sequence; identical to SEQ ID NO: 347
  Synthetic

SEQ ID NO: 349 - protein, 32 aa, Artificial Sequence
  Synthetic
RFTISRDNAKNTLYLQMNSLRPEDTAVYYCAA

SEQ ID NO: 350 - protein, 32 aa, Artificial Sequence; identical to SEQ ID NO: 349
  Synthetic

SEQ ID NO: 351 - protein, 32 aa, Artificial Sequence; identical to SEQ ID NO: 349
  Synthetic

SEQ ID NO: 352 - protein, 32 aa, Artificial Sequence; identical to SEQ ID NO: 346
  Synthetic

SEQ ID NO: 353 - protein, 32 aa, Artificial Sequence; identical to SEQ ID NO: 347
  Synthetic

SEQ ID NO: 354 - protein, 32 aa, Artificial Sequence; identical to SEQ ID NO: 347
  Synthetic

SEQ ID NO: 355 - protein, 32 aa, Artificial Sequence; identical to SEQ ID NO: 347
  Synthetic

SEQ ID NO: 356 - protein, 32 aa, Artificial Sequence; identical to SEQ ID NO: 349
  Synthetic

SEQ ID NO: 357 - protein, 32 aa, Artificial Sequence; identical to SEQ ID NO: 340
  Synthetic

SEQ ID NO: 358 - protein, 32 aa, Artificial Sequence; identical to SEQ ID NO: 340
  Synthetic

SEQ ID NO: 359 - protein, 32 aa, Artificial Sequence; identical to SEQ ID NO: 340
  Synthetic

SEQ ID NO: 360 - protein, 32 aa, Artificial Sequence; identical to SEQ ID NO: 346
  Synthetic

SEQ ID NO: 361 - protein, 32 aa, Artificial Sequence; identical to SEQ ID NO: 347
  Synthetic

SEQ ID NO: 362 - protein, 32 aa, Artificial Sequence; identical to SEQ ID NO: 347
  Synthetic

SEQ ID NO: 363 - protein, 32 aa, Artificial Sequence; identical to SEQ ID NO: 349
  Synthetic

SEQ ID NO: 364 - protein, 32 aa, Artificial Sequence; identical to SEQ ID NO: 349
  Synthetic

SEQ ID NO: 365 - protein, 32 aa, Artificial Sequence; identical to SEQ ID NO: 349
  Synthetic

SEQ ID NO: 366 - protein, 32 aa, Artificial Sequence
  Synthetic
RFTISRDNSKNTLYLQMNSLRAEDTAVYYCAA

SEQ ID NO: 367 - protein, 32 aa, Artificial Sequence; identical to SEQ ID NO: 349
  Synthetic

SEQ ID NO: 368 - protein, 32 aa, Artificial Sequence; identical to SEQ ID NO: 366
  Synthetic

SEQ ID NO: 369 - protein, 15 aa, Artificial Sequence
  Synthetic
HRTIATIPEKYEYEY

SEQ ID NO: 370 - protein, 15 aa, Artificial Sequence
  Synthetic
HRTIATIPEKYEYEY

SEQ ID NO: 371 - protein, 15 aa, Artificial Sequence
  Synthetic
QISAIVPISAHEYEY

SEQ ID NO: 372 - protein, 15 aa, Artificial Sequence
  Synthetic
HRGAIAPMTQSVYDY

SEQ ID NO: 373 - protein, 15 aa, Artificial Sequence
  Synthetic
HRGAIAPMTQSVYDT

SEQ ID NO: 374 - protein, 15 aa, Artificial Sequence
  Synthetic
HTTIATIPKKYEYEY

SEQ ID NO: 375 - protein, 15 aa, Artificial Sequence
  Synthetic
QISAIVPISAHEYQY

SEQ ID NO: 376 - protein, 15 aa, Artificial Sequence
  Synthetic
HLGAIAPMSQSVYDY

SEQ ID NO: 377 - protein, 15 aa, Artificial Sequence
  Synthetic
HRTIATIPEKYEYEY

SEQ ID NO: 378 - protein, 15 aa, Artificial Sequence
  Synthetic
HRGAIAPIAQSVYTN

SEQ ID NO: 379 - protein, 15 aa, Artificial Sequence
  Synthetic
HRTIATIPEKYEYEY

SEQ ID NO: 380 - protein, 15 aa, Artificial Sequence
  Synthetic
HRTIATIPEKYYYEY

SEQ ID NO: 381 - protein, 18 aa, Artificial Sequence
  Synthetic
DVPGTKIWSIQTPDRYNY

SEQ ID NO: 382 - protein, 10 aa, Artificial Sequence
  Synthetic
RDVAVAKYDS

SEQ ID NO: 383 - protein, 15 aa, Artificial Sequence
  Synthetic
HTTIATIPEKYEYEY

SEQ ID NO: 384 - protein, 17 aa, Artificial Sequence
  Synthetic
EVSARTGEHLPKLMGDY

SEQ ID NO: 385 - protein, 15 aa, Artificial Sequence
  Synthetic
HTTIATIPEKYEYEY

SEQ ID NO: 386 - protein, 15 aa, Artificial Sequence
  Synthetic
HRGAIAPMTQSVYDY

SEQ ID NO: 387 - protein, 15 aa, Artificial Sequence
  Synthetic
NRYNSDSRYMSSYDW

SEQ ID NO: 388 - protein, 15 aa, Artificial Sequence
  Synthetic
HRTIATIPEKYEYEY

SEQ ID NO: 389 - protein, 15 aa, Artificial Sequence
  Synthetic
HRTIATIPNKYEYDH

SEQ ID NO: 390 - protein, 15 aa, Artificial Sequence
  Synthetic
ALGAVVYTTREPYTY

SEQ ID NO: 391 - protein, 15 aa, Artificial Sequence
  Synthetic
HRTIATVPNKYEYDT

SEQ ID NO: 392 - protein, 12 aa, Artificial Sequence
  Synthetic
DFRDWTRRRYSY

SEQ ID NO: 393 - protein, 15 aa, Artificial Sequence
  Synthetic
GTDLSYYYSTKKWAY

SEQ ID NO: 394 - protein, 15 aa, Artificial Sequence
  Synthetic
HRGAIAPIAQSVYTN

SEQ ID NO: 395 - protein, 15 aa, Artificial Sequence
  Synthetic
HRGAIAPIAQSVYTN

SEQ ID NO: 396 - protein, 15 aa, Artificial Sequence
  Synthetic
HRGAIAPIAQSVYTN

SEQ ID NO: 397 - protein, 15 aa, Artificial Sequence
  Synthetic
HRGAIAPIAQSVYTN

SEQ ID NO: 398 - protein, 15 aa, Artificial Sequence
  Synthetic
HRGAIAPIAQSVYTN

SEQ ID NO: 399 - protein, 15 aa, Artificial Sequence
  Synthetic
HRGAIAPIAQSVYTN

SEQ ID NO: 400 - protein, 15 aa, Artificial Sequence
  Synthetic
HRGAIAPIAQSVYTN

SEQ ID NO: 401 - protein, 15 aa, Artificial Sequence
  Synthetic
HRGAIAPIAQSVYTN

SEQ ID NO: 402 - protein, 15 aa, Artificial Sequence
  Synthetic
HRTIATIPEKYEYEY

SEQ ID NO: 403 - protein, 15 aa, Artificial Sequence
  Synthetic
HRTIATIPEKYEYEY

SEQ ID NO: 404 - protein, 15 aa, Artificial Sequence
  Synthetic
HRTIATIPEKYEYEY

SEQ ID NO: 405 - protein, 15 aa, Artificial Sequence
  Synthetic
HRTIATIPEKYEYEY

SEQ ID NO: 406 - protein, 15 aa, Artificial Sequence
  Synthetic
HRTIATIPEKYEYEY

SEQ ID NO: 407 - protein, 15 aa, Artificial Sequence
  Synthetic
ALGAVVYTTREPYTY

SEQ ID NO: 408 - protein, 15 aa, Artificial Sequence
  Synthetic
ALGAVVYTTREPYTY

SEQ ID NO: 409 - protein, 15 aa, Artificial Sequence
  Synthetic
ALGAVVYTTREPYTY

SEQ ID NO: 410 - protein, 15 aa, Artificial Sequence
  Synthetic
ALGAVVYTTREPYTY

SEQ ID NO: 411 - protein, 15 aa, Artificial Sequence
  Synthetic
ALGAVVYTTREPYTY

SEQ ID NO: 412 - protein, 15 aa, Artificial Sequence
  Synthetic
ALGAVVYTTREPYTY

SEQ ID NO: 413 - protein, 15 aa, Artificial Sequence
  Synthetic
ALGAVVYTTREPYTY

SEQ ID NO: 414 - protein, 15 aa, Artificial Sequence
  Synthetic
ALGAVVYTTREPYTY

SEQ ID NO: 415 - protein, 15 aa, Artificial Sequence
  Synthetic
ALGAVVYTTREPYTY

SEQ ID NO: 416 - protein, 15 aa, Artificial Sequence
  Synthetic
HRTIATIPEKYEYEY

SEQ ID NO: 417 - protein, 15 aa, Artificial Sequence
  Synthetic
HTTIATIPEKYEYEY

SEQ ID NO: 418 - protein, 15 aa, Artificial Sequence
  Synthetic
HRTIATIPEKYEYEY

SEQ ID NO: 419 - protein, 11 aa, Artificial Sequence
  Synthetic
WGQGTQVTVSS

SEQ ID NO: 420 - protein, 11 aa, Artificial Sequence
  Synthetic
WGQGTQVTVSS

SEQ ID NO: 421 - protein, 11 aa, Artificial Sequence
  Synthetic
WGQGTQVTVSS

SEQ ID NO: 422 - protein, 11 aa, Artificial Sequence
  Synthetic
WGQGTQVTVSS

SEQ ID NO: 423 - protein, 11 aa, Artificial Sequence
  Synthetic
WGQGTQVTVSS

SEQ ID NO: 424 - protein, 11 aa, Artificial Sequence
  Synthetic
WGQGTQVTVSS

SEQ ID NO: 425 - protein, 11 aa, Artificial Sequence
  Synthetic
WGQGTQVTVSS

SEQ ID NO: 426 - protein, 11 aa, Artificial Sequence
  Synthetic
WGQGTQVTVSS

SEQ ID NO: 427 - protein, 11 aa, Artificial Sequence
  Synthetic
WGQGTQVTVSS

SEQ ID NO: 428 - protein, 11 aa, Artificial Sequence
  Synthetic
WGQGTQVTVSS

SEQ ID NO: 429 - protein, 11 aa, Artificial Sequence
  Synthetic
WGQGTQVTVSS

SEQ ID NO: 430 - protein, 11 aa, Artificial Sequence
  Synthetic
WGQGTQVTVSS

SEQ ID NO: 431 - protein, 11 aa, Artificial Sequence
  Synthetic
WGQGTQVTVSS

SEQ ID NO: 432 - protein, 11 aa, Artificial Sequence
  Synthetic
WGQGTQVTVSS

SEQ ID NO: 433 - protein, 11 aa, Artificial Sequence
  Synthetic
WGRGTQVTVSS

SEQ ID NO: 434 - protein, 11 aa, Artificial Sequence
  Synthetic
WGQGTQVTVSS

SEQ ID NO: 435 - protein, 11 aa, Artificial Sequence
  Synthetic
WGQGTQVTVSS

SEQ ID NO: 436 - protein, 11 aa, Artificial Sequence
  Synthetic
WGQGTQVTVSS

SEQ ID NO: 437 - protein, 11 aa, Artificial Sequence
  Synthetic
WGQGTQVTVSS

SEQ ID NO: 438 - protein, 11 aa, Artificial Sequence
  Synthetic
WGQGTQVTVSS

SEQ ID NO: 439 - protein, 11 aa, Artificial Sequence
  Synthetic
WGQGTQVTVSS

SEQ ID NO: 440 - protein, 11 aa, Artificial Sequence
  Synthetic
WGRGTQVTVSS

SEQ ID NO: 441 - protein, 11 aa, Artificial Sequence
  Synthetic
WGQGTQVTVSS

SEQ ID NO: 442 - protein, 11 aa, Artificial Sequence
  Synthetic
WGQGTQVTVSS

SEQ ID NO: 443 - protein, 11 aa, Artificial Sequence
  Synthetic
WGQGTQVTVSS

SEQ ID NO: 444 - protein, 11 aa, Artificial Sequence
  Synthetic
WGQGTQVTVSS

SEQ ID NO: 445 - protein, 11 aa, Artificial Sequence
  Synthetic
WGQGTQVTVSS

SEQ ID NO: 446 - protein, 11 aa, Artificial Sequence
  Synthetic
WGQGTLVTVSS

SEQ ID NO: 447 - protein, 11 aa, Artificial Sequence
  Synthetic
WGQGTLVTVSS

SEQ ID NO: 448 - protein, 11 aa, Artificial Sequence
  Synthetic
WGQGTLVTVSS

SEQ ID NO: 449 - protein, 11 aa, Artificial Sequence
  Synthetic
WGQGTLVTVSS

SEQ ID NO: 450 - protein, 11 aa, Artificial Sequence
  Synthetic
WGQGTLVTVSS

SEQ ID NO: 451 - protein, 11 aa, Artificial Sequence
  Synthetic
WGQGTLVTVSS

SEQ ID NO: 452 - protein, 11 aa, Artificial Sequence
  Synthetic
WGQGTLVTVSS

SEQ ID NO: 453 - protein, 11 aa, Artificial Sequence
  Synthetic
WGQGTLVTVSS

SEQ ID NO: 454 - protein, 11 aa, Artificial Sequence
  Synthetic
WGQGTLVTVSS

SEQ ID NO: 455 - protein, 11 aa, Artificial Sequence
  Synthetic
WGQGTLVTVSS

SEQ ID NO: 456 - protein, 11 aa, Artificial Sequence
  Synthetic
WGQGTLVTVSS

SEQ ID NO: 457 - protein, 11 aa, Artificial Sequence
  Synthetic
WGRGTQVTVSS

SEQ ID NO: 458 - protein, 11 aa, Artificial Sequence
  Synthetic
WGRGTQVTVSS

SEQ ID NO: 459 - protein, 11 aa, Artificial Sequence
  Synthetic
WGRGTQVTVSS

SEQ ID NO: 460 - protein, 11 aa, Artificial Sequence
  Synthetic
WGQGTLVTVSS

SEQ ID NO: 461 - protein, 11 aa, Artificial Sequence
  Synthetic
WGQGTLVTVSS

SEQ ID NO: 462 - protein, 11 aa, Artificial Sequence
  Synthetic
WGQGTLVTVSS

SEQ ID NO: 463 - protein, 11 aa, Artificial Sequence
  Synthetic
WGQGTLVTVSS

SEQ ID NO: 464 - protein, 11 aa, Artificial Sequence
  Synthetic
WGQGTLVTVSS

SEQ ID NO: 465 - protein, 11 aa, Artificial Sequence
  Synthetic
WGQGTLVTVSS

SEQ ID NO: 466 - protein, 11 aa, Artificial Sequence
  Synthetic
WGQGTLVTVSS

SEQ ID NO: 467 - protein, 11 aa, Artificial Sequence
  Synthetic
WGQGTLVTVSS

SEQ ID NO: 468 - protein, 11 aa, Artificial Sequence
  Synthetic
WGQGTLVTVSS

SEQ ID NO: 469 - protein, 124 aa, Artificial Sequence
  Synthetic
DVQLVESGGGLVQAGDSLRLSCAASGRTFISYAVGWFRQAPGSEREFVAGIRWNGIHTDYADSVKGRFTISRDNAKNTVTLEMTSLKPEDTAVYYCAAHRTIATIPEKYEYEYWGQGTQVTVSS

SEQ ID NO: 470 - protein, 124 aa, Artificial Sequence
  Synthetic
EVQLVESGGRLVRAGDSLRLSCAASGRTFLSYAVGWFRQAPGTEREFVAGIRWSGGYTDYAEAVKGRFTISRDNAKNTVYLQMNSLKPEDTAVYYCAAHRTIATIPEKYEYEYWGQGTQVTVSS

SEQ ID NO: 471 - protein, 124 aa, Artificial Sequence
  Synthetic
QVQLVESGGGLVQAGGSLRLACSASGRTFLTYALGWFRQAPGKEREFVAGVSWSGSGTKYADSVKGRFTISRDNAKNTVYMQMNSLKPEDTAVYYCAAQISAIVPISAHEYEYWGQGTQVTVSS

SEQ ID NO: 472 - protein, 124 aa, Artificial Sequence
  Synthetic
QVQLVESGGGLVQAGGSLRLSCAASGRTFITYAIGWFRQAPGKEREFVTAINWSGSMTSYADSVKGRFTISRDNNKNMVYLQMNSLKPEDTAVYYCAAHRGAIAPMTQSVYDYWGQGTQVTVSS

SEQ ID NO: 473 - protein, 124 aa, Artificial Sequence
  Synthetic
AVQLVESGGGLVQAGGSLRLSCAASGRTFITYAIGWFRQAPGKEREFVSAINWSGSMTSYADSVKGRFTISRDNAKNTVYLQMNGLKPEDTAVYYCAAHRGAIAPMTQSVYDTWGQGTQVTVSS

SEQ ID NO: 474 - protein, 124 aa, Artificial Sequence
  Synthetic
AVQLVESGGGLVQAGDSLRLSCTASGRTFLSYAVGWFRQAPGTEREFVAGIRWSGEHTDYAASVKGRFTISRDNAKNTVYLQMNSLKPEDTAVYYCAAHTTIATIPKKYEYEYWGQGTQVTVSS

SEQ ID NO: 475 - protein, 124 aa, Artificial Sequence
  Synthetic
AVQLVESGGGLVQAGDSLRLACAASGRTFLTYALGWFRQAPGKEREFVAGVSWSGSSTKYADSVKGRFTISRDNAKNTVYMQMNSLKPEDTAVYYCAAQISAIVPISAHEYQYWGQGTQVTVSS

SEQ ID NO: 476 - protein, 124 aa, Artificial Sequence
  Synthetic
EVQLVESGGGLVRAGGSLRLSCAASGRTFITYAIGWFRQAPGKEREFVTAINWSGSMTSYADSVKGRFTISRDNNKNTVYLQMNSLKPEDTALYYCAAHLGAIAPMSQSVYDYWGQGTQVTVSS

SEQ ID NO: 477 - protein, 124 aa, Artificial Sequence
  Synthetic
EVQLVESGGGLVQAGDSLRLSCTASGRTFLSYAVGWFRQAPGTEREFVAGIRWSGGSTDYSDSVKGRFTISRDNAKNTVYLQMNSLKPEDTAVYYCAAHRTIATIPEKYEYEYWGQGTQVTVSS

SEQ ID NO: 478 - protein, 124 aa, Artificial Sequence
  Synthetic
QVQLVESGGGLVQAGGSLRLSCAASGRTFITYAIGWFRQAPGKEREFVSAINWSGSMTSYADSVKGRFTISRDNAKNTVYLQMNGLKPEDTAVYYCAAHRGAIAPIAQSVYTNWGQGTQVTVSS

SEQ ID NO: 479 - protein, 124 aa, Artificial Sequence
  Synthetic
AVQLVESGGGLVQAGDSLRLSCTASGRTFLTYAVGWFRQAPGTEREFVAGIRWSGGYTDYADSVKGRFTISRDNAKNTVYLQMNSLKPEDTAVYYCAAHRTIATIPEKYEYEYWGQGTQVTVSS

SEQ ID NO: 480 - protein, 124 aa, Artificial Sequence
  Synthetic
EVQLVESGGGLVQAGDSLRLSCAASGRTFLTYAVGWFRQAPGTEREFVAGIRWSGESTDYAESVKGRFTISRDDTKNTVYLQMNSLKPEDTAVYYCAAHRTIATIPEKYYYEYWGQGTQVTVSS

SEQ ID NO: 481 - protein, 127 aa, Artificial Sequence
  Synthetic
QVQLVESGGGLVQAGGSLRLSCAVSGRPVSSAVMGWFRQAPGKEREFVGRLTSSATSTFYAESVKGRFTISRDNAKNTVYLQMNNLKPEDTAVYYCAADVPGTKIWSIQTPDRYNYWGQGTQVTVSS

SEQ ID NO: 482 - protein, 119 aa, Artificial Sequence
  Synthetic
EVQLVESGGGLVQAGGSLSLSCAVSGRTLTGLLIGWFRQAPGKERELVAIISWTYGSTNYADSVKGRFTISRDNAKNTVLLQMNSLKPEDTAVYYCSARDVAVAKYDSWGQGTQVTVSS

SEQ ID NO: 483 - protein, 124 aa, Artificial Sequence
  Synthetic
QVQLVESGGGLVQAGDSLRLSCAASGRTFLTLAVGWFRQAPGTEREFVAGIRWSGSGTDYADSVKGRFTISRDNAKNTVYLQMNSLKPEDTAVYYCAAHTTIATIPEKYEYEYWGRGTQVTVSS

SEQ ID NO: 484 - protein, 126 aa, Artificial Sequence
  Synthetic
QVQLVESGGGLVQAGGSLKLSCAASGRTFITYAMGWFRQAPGKERELVAAISWSGSSTYSADSVKGRFTISRDNAKNTVYLQMNSLKPEDTAVYYCAAEVSARTGEHLPKLMGDYWGQGTQVTVSS

SEQ ID NO: 485 - protein, 124 aa, Artificial Sequence
  Synthetic
QVQLVESGGGLVQAGDSLRLSCAASGRTFLTLAVGWFRQAPGTEREFVAGIRWSGSGTDYADSVKGRFTISRDNAANTVYLQMNSLKPEDTAVYYCAAHTTIATIPEKYEYEYWGQGTQVTVSS

SEQ ID NO: 486 - protein, 124 aa, Artificial Sequence
  Synthetic
QVQLVESGGGLVQAGGSLRLSCAASGRTFITYAIGWFRQAPGKEREFVTAINWSGSMTSYADSVKGRFTISRDNAKNTVYLQINGLKSEDTAVYYCAAHRGAIAPMTQSVYDYWGQGTQVTVSS

SEQ ID NO: 487 - protein, 124 aa, Artificial Sequence
  Synthetic
QVKLEESGGGLVQAGGSLRLSCVASGRTFSRYAMGWFRQAPGKEREFVSTSTGSGGLTSYANSVKGRFTISRDNAKNTVYLQMNNLKPEDTAIYYCAANRYNSDSRYMSSYDWWGQGTQVTVSS

SEQ ID NO: 488 - protein, 124 aa, Artificial Sequence
  Synthetic
AVQLVESGGGLVQAGDSLRLSCAASGRTFLSYAVGWFRQAPGTEREFVAGIRWSGLHTDYADSVKGRFTISRDNAKNTVYLQMNSLKPEDTAIYYCAAHRTIATIPEKYEYEYWGQGTQVTVSS

SEQ ID NO: 489 - protein, 124 aa, Artificial Sequence
  Synthetic
DVQLVESGGGLVQAGDSLRLTCSASGRTFISYAVGWFRQAPGTEREFVAGIRWNGISTDYTDSVKGRFTISRDNAKNTVYLQMNSLKPEDTAIYYCAAHRTIATIPNKYEYDHWGQGTQVTVSS

SEQ ID NO: 490 - protein, 124 aa, Artificial Sequence
  Synthetic
EVQLVESGGGLVQAGDSLRLSCVASGRTFVTYGMGWFRQAPGKEREFVAAINWSGSMTSYGDSVKGRFAISRDNAKNTVYLQMNSLKPEDTAVYYCAAALGAVVYTTREPYTYWGRGTQVTVSS

SEQ ID NO: 491 - protein, 124 aa, Artificial Sequence
  Synthetic
EVQLVESGGGLVQAGDSLRLSCAASGRTFLSYAVGWFRQAPGTEREFVAGIRWSGGSTDYADSVKGRFTISRDNAKNTVYLQMNSLKPEDTAVYYCAAHRTIATVPNKYEYDTWGQGTQVTVSS

SEQ ID NO: 492 - protein, 120 aa, Artificial Sequence
  Synthetic
QVKLEESGGGLVQVGDSLRLSCAASVSSFSINDMGWYRQAPGKQRELVATIASGGSTNYADSVKGRFTISRDNVKNTVYLQMNGLKPEDTAVYYCNADFRDWTRRRYSYWGQGTQVTVSS

SEQ ID NO: 493 - protein, 124 aa, Artificial Sequence
  Synthetic
QVKLEESGGGLVQVGDSLRLSCAASGRTFSNYTMAWFRQFPGKEREFVAVVSRGGGATDYADSVKGRFTISRDNAKNTMYLQMNSLKTEDTAVYYCAAGTDLSYYYSTKKWAYWGQGTQVTVSS

SEQ ID NO: 494 - protein, 124 aa, Artificial Sequence
  Synthetic
QVQLVESGGGLVQAGGSLRLSCAASGRTFITYAIGWFRQAPGKEREFVSAISWSGSMTSYADSVKGRFTISRDNAKNTVYLQMNGLKPEDTAVYYCAAHRGAIAPIAQSVYTNWGQGTQVTVSS

SEQ ID NO: 495 - protein, 124 aa, Artificial Sequence
  Synthetic
QVQLVESGGGLVQAGGSLRLSCAASGRTFITYAIGWFRQAPGKEREFVSAIQWSGSMTSYADSVKGRFTISRDNAKNTVYLQMNGLKPEDTAVYYCAAHRGAIAPIAQSVYTNWGQGTQVTVSS

SEQ ID NO: 496 - protein, 124 aa, Artificial Sequence
  Synthetic
EVQLVESGGGLVQPGGSLRLSCAASGRTFITYAIGWVRQAPGKGLEWVSAIQWSGSMTSYADSVKGRFTISRDNSKNTLYLQMNSLRAEDTAVYYCAKHRGAIAPIAQSVYTNWGQGTLVTVSS

SEQ ID NO: 497 - protein, 124 aa, Artificial Sequence
  Synthetic
EVQLVESGGGLVQPGGSLRLSCAASGRTFITYAIGWFRQAPGKGLEWVSAIQWSGSMTSYADSVKGRFTISRDNSKNTVYLQMNSLRAEDTAVYYCAAHRGAIAPIAQSVYTNWGQGTLVTVSS

SEQ ID NO: 498 - protein, 124 aa, Artificial Sequence
  Synthetic
EVQLVESGGGLVQPGGSLRLSCAASGRTFITYAIGWFRQAPGKGLEFVSAIQWSGSMTSYADSVKGRFTISRDNSKNTVYLQMNSLRAEDTAVYYCAAHRGAIAPIAQSVYTNWGQGTLVTVSS

SEQ ID NO: 499 - protein, 124 aa, Artificial Sequence
  Synthetic
EVQLVESGGGLVQPGGSLRLSCAASGRTFITYAIGWFRQAPGKGREFVSAISWSGSMTSYADSVKGRFTISRDNAKNTLYLQMNSLRPEDTAVYYCAAHRGAIAPIAQSVYTNWGQGTLVTVSS

SEQ ID NO: 500 - protein, 124 aa, Artificial Sequence
  Synthetic
EVQLVESGGGLVQPGGSLRLSCAASGRTFITYAIGWFRQAPGKGREFVSAISWSGSITSYADSVKGRFTISRDNAKNTLYLQMNSLRPEDTAVYYCAAHRGAIAPIAQSVYTNWGQGTLVTVSS

SEQ ID NO: 501 - protein, 124 aa, Artificial Sequence
  Synthetic
EVQLVESGGGLVQPGGSLRLSCAASGRTFITYAIGWFRQAPGKGREFVSAISWSGSLTSYADSVKGRFTISRDNAKNTLYLQMNSLRPEDTAVYYCAAHRGAIAPIAQSVYTNWGQGTLVTVSS

SEQ ID NO: 502 - protein, 124 aa, Artificial Sequence
  Synthetic
EVQLVESGGGLVQPGGSLRLSCAASGRTFLTYAVGWVRQAPGKGLEWVSGIRWSGGYTDYADSVKGRFTISRDNSKNTLYLQMNSLRAEDTAVYYCAKHRTIATIPEKYEYEYWGQGTLVTVSS

SEQ ID NO: 503 - protein, 124 aa, Artificial Sequence
  Synthetic
EVQLVESGGGLVQPGGSLRLSCAASGRTFLTYAVGWFRQAPGKGLEWVAGIRWSGGYTDYADSVKGRFTISRDNSKNTVYLQMNSLRAEDTAVYYCAAHRTIATIPEKYEYEYWGQGTLVTVSS

SEQ ID NO: 504 - protein, 124 aa, Artificial Sequence
  Synthetic
EVQLVESGGGLVQPGGSLRLSCAASGRTFLTYAVGWFRQAPGKGLEFVAGIRWSGGYTDYADSVKGRFTISRDNSKNTVYLQMNSLRAEDTAVYYCAAHRTIATIPEKYEYEYWGQGTLVTVSS

SEQ ID NO: 505 - protein, 124 aa, Artificial Sequence
  Synthetic
EVQLVESGGGLVQPGGSLRLSCTASGRTFLTYAVGWFRQAPGKGLEFVAGIRWSGGYTDYADSVKGRFTISRDNSKNTVYLQMNSLRAEDTAVYYCAAHRTIATIPEKYEYEYWGQGTLVTVSS

SEQ ID NO: 506 - protein, 124 aa, Artificial Sequence
  Synthetic
EVQLVESGGGLVQPGGSLRLSCAASGRTFLTYAVGWFRQAPGKGREFVSGIRWSGGYTDYADSVKGRFTISRDNAKNTLYLQMNSLRPEDTAVYYCAAHRTIATIPEKYEYEYWGQGTLVTVSS

SEQ ID NO: 507 - protein, 124 aa, Artificial Sequence
  Synthetic
EVQLVESGGGLVQAGDSLRLSCVASGRTFVTYGMGWFRQAPGKEREFVAAIAWSGSMTSYGDSVKGRFAISRDNAKNTVYLQMNSLKPEDTAVYYCAAALGAVVYTTREPYTYWGRGTQVTVSS

SEQ ID NO: 508 - protein, 124 aa, Artificial Sequence
  Synthetic
EVQLVESGGGLVQAGDSLRLSCVASGRTFVTYGMGWFRQAPGKEREFVAAISWSGSMTSYGDSVKGRFAISRDNAKNTVYLQMNSLKPEDTAVYYCAAALGAVVYTTREPYTYWGRGTQVTVSS

SEQ ID NO: 509 - protein, 124 aa, Artificial Sequence
  Synthetic
EVQLVESGGGLVQAGDSLRLSCVASGRTFVTYGMGWFRQAPGKEREFVAAIQWSGSMTSYGDSVKGRFAISRDNAKNTVYLQMNSLKPEDTAVYYCAAALGAVVYTTREPYTYWGRGTQVTVSS

SEQ ID NO: 510 - protein, 124 aa, Artificial Sequence
  Synthetic
EVQLVESGGGLVQPGGSLRLSCAASGRTFVTYGMGWVRQAPGKGLEWVSAIQWSGSMTSYGDSVKGRFTISRDNSKNTLYLQMNSLRAEDTAVYYCAKALGAVVYTTREPYTYWGQGTLVTVSS

SEQ ID NO: 511 - protein, 124 aa, Artificial Sequence
  Synthetic
EVQLVESGGGLVQPGGSLRLSCAASGRTFVTYGMGWFRQAPGKGLEFVAAIQWSGSMTSYGDSVKGRFTISRDNSKNTVYLQMNSLRAEDTAVYYCAAALGAVVYTTREPYTYWGQGTLVTVSS

SEQ ID NO: 512 - protein, 124 aa, Artificial Sequence
  Synthetic
EVQLVESGGGLVQPGGSLRLSCVASGRTFVTYGMGWFRQAPGKGREFVSAIQWSGSMTSYGDSVKGRFTISRDNSKNTVYLQMNSLRAEDTAVYYCAAALGAVVYTTREPYTYWGQGTLVTVSS

SEQ ID NO: 513 - protein, 124 aa, Artificial Sequence
  Synthetic
EVQLVESGGGLVQPGGSLRLSCAASGRTFVTYGMGWFRQAPGKGREFVSAISWSGSMTSYGDSVKGRFTISRDNAKNTLYLQMNSLRPEDTAVYYCAAALGAVVYTTREPYTYWGQGTLVTVSS

SEQ ID NO: 514 - protein, 124 aa, Artificial Sequence
  Synthetic
EVQLVESGGGLVQPGGSLRLSCAASGRTFVTYGMGWFRQAPGKGREFVSAISWSGSITSYGDSVKGRFTISRDNAKNTLYLQMNSLRPEDTAVYYCAAALGAVVYTTREPYTYWGQGTLVTVSS

SEQ ID NO: 515 - protein, 124 aa, Artificial Sequence
  Synthetic
EVQLVESGGGLVQPGGSLRLSCAASGRTFVTYGMGWFRQAPGKGREFVSAISWSGSLTSYGDSVKGRFTISRDNAKNTLYLQMNSLRPEDTAVYYCAAALGAVVYTTREPYTYWGQGTLVTVSS

SEQ ID NO: 516 - protein, 124 aa, Artificial Sequence
  Synthetic
EVQLVESGGGLVQPGGSLRLSCAASGRTFISYAVGWFRQAPGKGREFVSGIRWSGIHTDYADSVKGRFTISRDNAKNTLYLQMNSLRPEDTAVYYCAAHRTIATIPEKYEYEYWGQGTLVTVSS

SEQ ID NO: 517 - protein, 124 aa, Artificial Sequence
  Synthetic
EVQLVESGGGLVQPGGSLRLSCAASGRTFLTLAVGWFRQAPGKGREFVSGIRWSGSGTDYADSVKGRFTISRDNAKNTLYLQMNSLRPEDTAVYYCAAHTTIATIPEKYEYEYWGQGTLVTVSS

SEQ ID NO: 518 - protein, 124 aa, Artificial Sequence
  Synthetic
EVQLVESGGGLVQPGGSLRLSCAASGRTFLTYAVGWFRQAPGKGLEFVSGIRWSGGYTDYADSVKGRFTISRDNSKNTLYLQMNSLRAEDTAVYYCAAHRTIATIPEKYEYEYWGQGTLVTVSS

SEQ ID NO: 519 - protein, 356 aa, Artificial Sequence
  Synthetic
DVQLVESGGGLVQAGDSLRLSCAASGRTFISYAVGWFRQAPGSEREFVAGIRWNGIHTDYADSVKGRFTISRDNAKNTVTLEMTSLKPEDTAVYYCAAHRTIATIPEKYEYEYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 520 - protein, 356 aa, Artificial Sequence
  Synthetic
EVQLVESGGGLVQPGGSLRLSCAASGRTFISYAVGWFRQAPGKGREFVSGIRWSGIHTDYADSVKGRFTISRDNAKNTLYLQMNSLRPEDTAVYYCAAHRTIATIPEKYEYEYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 521 - protein, 356 aa, Artificial Sequence
  Synthetic
EVQLVESGGRLVRAGDSLRLSCAASGRTFLSYAVGWFRQAPGTEREFVAGIRWSGGYTDYAEAVKGRFTISRDNAKNTVYLQMNSLKPEDTAVYYCAAHRTIATIPEKYEYEYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 522 - protein, 356 aa, Artificial Sequence
  Synthetic
QVQLVESGGGLVQAGGSLRLSCAASGRTFITYAIGWFRQAPGKEREFVTAINWSGSMTSYADSVKGRFTISRDNNKNMVYLQMNSLKPEDTAVYYCAAHRGAIAPMTQSVYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 523 - protein, 356 aa, Artificial Sequence
  Synthetic
AVQLVESGGGLVQAGDSLRLSCTASGRTFLSYAVGWFRQAPGTEREFVAGIRWSGEHTDYAASVKGRFTISRDNAKNTVYLQMNSLKPEDTAVYYCAAHTTIATIPKKYEYEYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 524 - protein, 356 aa, Artificial Sequence
  Synthetic
AVQLVESGGGLVQAGDSLRLACAASGRTFLTYALGWFRQAPGKEREFVAGVSWSGSSTKYADSVKGRFTISRDNAKNTVYMQMNSLKPEDTAVYYCAAQISAIVPISAHEYQYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 525 - protein, 356 aa, Artificial Sequence
  Synthetic
EVQLVESGGGLVRAGGSLRLSCAASGRTFITYAIGWFRQAPGKEREFVTAINWSGSMTSYADSVKGRFTISRDNNKNTVYLQMNSLKPEDTALYYCAAHLGAIAPMSQSVYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 526 - protein, 356 aa, Artificial Sequence
  Synthetic
QVQLVESGGGLVQAGGSLRLSCAASGRTFITYAIGWFRQAPGKEREFVSAINWSGSMTSYADSVKGRFTISRDNAKNTVYLQMNGLKPEDTAVYYCAAHRGAIAPIAQSVYTNWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 527 - protein, 356 aa, Artificial Sequence
  Synthetic
AVQLVESGGGLVQAGDSLRLSCTASGRTFLTYAVGWFRQAPGTEREFVAGIRWSGGYTDYADSVKGRFTISRDNAKNTVYLQMNSLKPEDTAVYYCAAHRTIATIPEKYEYEYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 528 - protein, 356 aa, Artificial Sequence
  Synthetic
QVQLVESGGGLVQAGDSLRLSCAASGRTFLTLAVGWFRQAPGTEREFVAGIRWSGSGTDYADSVKGRFTISRDNAKNTVYLQMNSLKPEDTAVYYCAAHTTIATIPEKYEYEYWGRGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 529 - protein, 356 aa, Artificial Sequence
  Synthetic
EVQLVESGGGLVQPGGSLRLSCAASGRTFLTLAVGWFRQAPGKGREFVSGIRWSGSGTDYADSVKGRFTISRDNAKNTLYLQMNSLRPEDTAVYYCAAHTTIATIPEKYEYEYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 530 - protein, 356 aa, Artificial Sequence
  Synthetic
EVQLVESGGGLVQPGGSLRLSCAASGRTFLTYAVGWFRQAPGKGLEFVSGIRWSGGYTDYADSVKGRFTISRDNSKNTLYLQMNSLRAEDTAVYYCAAHRTIATIPEKYEYEYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 531 - protein, 358 aa, Artificial Sequence
  Synthetic
QVQLVESGGGLVQAGGSLKLSCAASGRTFITYAMGWFRQAPGKERELVAAISWSGSSTYSADSVKGRFTISRDNAKNTVYLQMNSLKPEDTAVYYCAAEVSARTGEHLPKLMGDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 532 - protein, 356 aa, Artificial Sequence
  Synthetic
QVQLVESGGGLVQAGGSLRLSCAASGRTFITYAIGWFRQAPGKEREFVTAINWSGSMTSYADSVKGRFTISRDNAKNTVYLQINGLKSEDTAVYYCAAHRGAIAPMTQSVYDYWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 533 - protein, 356 aa, Artificial Sequence
  Synthetic
DVQLVESGGGLVQAGDSLRLTCSASGRTFISYAVGWFRQAPGTEREFVAGIRWNGISTDYTDSVKGRFTISRDNAKNTVYLQMNSLKPEDTAIYYCAAHRTIATIPNKYEYDHWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 534 - protein, 356 aa, Artificial Sequence
  Synthetic
EVQLVESGGGLVQAGDSLRLSCVASGRTFVTYGMGWFRQAPGKEREFVAAINWSGSMTSYGDSVKGRFAISRDNAKNTVYLQMNSLKPEDTAVYYCAAALGAVVYTTREPYTYWGRGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 535 - protein, 356 aa, Artificial Sequence
  Synthetic
EVQLVESGGGLVQAGDSLRLSCAASGRTFLSYAVGWFRQAPGTEREFVAGIRWSGGSTDYADSVKGRFTISRDNAKNTVYLQMNSLKPEDTAVYYCAAHRTIATVPNKYEYDTWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 536 - protein, 356 aa, Artificial Sequence
  Synthetic
QVQLVESGGGLVQAGGSLRLSCAASGRTFITYAIGWFRQAPGKEREFVSAIAWSGSMTSYADSVKGRFTISRDNAKNTVYLQMNGLKPEDTAVYYCAAHRGAIAPIAQSVYTNWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 537 - protein, 356 aa, Artificial Sequence
  Synthetic
QVQLVESGGGLVQAGGSLRLSCAASGRTFITYAIGWFRQAPGKEREFVSAISWSGSMTSYADSVKGRFTISRDNAKNTVYLQMNGLKPEDTAVYYCAAHRGAIAPIAQSVYTNWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 538 - protein, 356 aa, Artificial Sequence
  Synthetic
QVQLVESGGGLVQAGGSLRLSCAASGRTFITYAIGWFRQAPGKEREFVSAIQWSGSMTSYADSVKGRFTISRDNAKNTVYLQMNGLKPEDTAVYYCAAHRGAIAPIAQSVYTNWGQGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 539 - protein, 356 aa, Artificial Sequence
  Synthetic
EVQLVESGGGLVQPGGSLRLSCAASGRTFITYAIGWVRQAPGKGLEWVSAIQWSGSMTSYADSVKGRFTISRDNSKNTLYLQMNSLRAEDTAVYYCAKHRGAIAPIAQSVYTNWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 540 - protein, 356 aa, Artificial Sequence
  Synthetic
EVQLVESGGGLVQPGGSLRLSCAASGRTFITYAIGWFRQAPGKGLEWVSAIQWSGSMTSYADSVKGRFTISRDNSKNTVYLQMNSLRAEDTAVYYCAAHRGAIAPIAQSVYTNWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 541 - protein, 356 aa, Artificial Sequence
  Synthetic
EVQLVESGGGLVQPGGSLRLSCAASGRTFITYAIGWFRQAPGKGLEFVSAIQWSGSMTSYADSVKGRFTISRDNSKNTVYLQMNSLRAEDTAVYYCAAHRGAIAPIAQSVYTNWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 542 - protein, 356 aa, Artificial Sequence
  Synthetic
EVQLVESGGGLVQPGGSLRLSCAASGRTFITYAIGWFRQAPGKGREFVSAISWSGSMTSYADSVKGRFTISRDNAKNTLYLQMNSLRPEDTAVYYCAAHRGAIAPIAQSVYTNWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 543 - protein, 356 aa, Artificial Sequence
  Synthetic
EVQLVESGGGLVQPGGSLRLSCAASGRTFITYAIGWFRQAPGKGREFVSAISWSGSITSYADSVKGRFTISRDNAKNTLYLQMNSLRPEDTAVYYCAAHRGAIAPIAQSVYTNWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 544 - protein, 356 aa, Artificial Sequence
  Synthetic
EVQLVESGGGLVQPGGSLRLSCAASGRTFITYAIGWFRQAPGKGREFVSAISWSGSLTSYADSVKGRFTISRDNAKNTLYLQMNSLRPEDTAVYYCAAHRGAIAPIAQSVYTNWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 545 - protein, 356 aa, Artificial Sequence
  Synthetic
EVQLVESGGGLVQPGGSLRLSCAASGRTFLTYAVGWVRQAPGKGLEWVSGIRWSGGYTDYADSVKGRFTISRDNSKNTLYLQMNSLRAEDTAVYYCAKHRTIATIPEKYEYEYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 546 - protein, 356 aa, Artificial Sequence
  Synthetic
EVQLVESGGGLVQPGGSLRLSCAASGRTFLTYAVGWFRQAPGKGLEWVAGIRWSGGYTDYADSVKGRFTISRDNSKNTVYLQMNSLRAEDTAVYYCAAHRTIATIPEKYEYEYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 547 - protein, 356 aa, Artificial Sequence
  Synthetic
EVQLVESGGGLVQPGGSLRLSCAASGRTFLTYAVGWFRQAPGKGLEFVAGIRWSGGYTDYADSVKGRFTISRDNSKNTVYLQMNSLRAEDTAVYYCAAHRTIATIPEKYEYEYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 548 - protein, 356 aa, Artificial Sequence
  Synthetic
EVQLVESGGGLVQPGGSLRLSCTASGRTFLTYAVGWFRQAPGKGLEFVAGIRWSGGYTDYADSVKGRFTISRDNSKNTVYLQMNSLRAEDTAVYYCAAHRTIATIPEKYEYEYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 549 - protein, 356 aa, Artificial Sequence
  Synthetic
EVQLVESGGGLVQPGGSLRLSCAASGRTFLTYAVGWFRQAPGKGREFVSGIRWSGGYTDYADSVKGRFTISRDNAKNTLYLQMNSLRPEDTAVYYCAAHRTIATIPEKYEYEYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 550 - protein, 356 aa, Artificial Sequence
  Synthetic
EVQLVESGGGLVQAGDSLRLSCVASGRTFVTYGMGWFRQAPGKEREFVAAIAWSGSMTSYGDSVKGRFAISRDNAKNTVYLQMNSLKPEDTAVYYCAAALGAVVYTTREPYTYWGRGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 551 - protein, 356 aa, Artificial Sequence
  Synthetic
EVQLVESGGGLVQAGDSLRLSCVASGRTFVTYGMGWFRQAPGKEREFVAAISWSGSMTSYGDSVKGRFAISRDNAKNTVYLQMNSLKPEDTAVYYCAAALGAVVYTTREPYTYWGRGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 552 - protein, 356 aa, Artificial Sequence
  Synthetic
EVQLVESGGGLVQAGDSLRLSCVASGRTFVTYGMGWFRQAPGKEREFVAAIQWSGSMTSYGDSVKGRFAISRDNAKNTVYLQMNSLKPEDTAVYYCAAALGAVVYTTREPYTYWGRGTQVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 553 - protein, 356 aa, Artificial Sequence
  Synthetic
EVQLVESGGGLVQPGGSLRLSCAASGRTFVTYGMGWVRQAPGKGLEWVSAIQWSGSMTSYGDSVKGRFTISRDNSKNTLYLQMNSLRAEDTAVYYCAKALGAVVYTTREPYTYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 554 - protein, 356 aa, Artificial Sequence
  Synthetic
EVQLVESGGGLVQPGGSLRLSCAASGRTFVTYGMGWFRQAPGKGLEFVAAIQWSGSMTSYGDSVKGRFTISRDNSKNTVYLQMNSLRAEDTAVYYCAAALGAVVYTTREPYTYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 555 - protein, 356 aa, Artificial Sequence
  Synthetic
EVQLVESGGGLVQPGGSLRLSCVASGRTFVTYGMGWFRQAPGKGREFVSAIQWSGSMTSYGDSVKGRFTISRDNSKNTVYLQMNSLRAEDTAVYYCAAALGAVVYTTREPYTYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 556 - protein, 356 aa, Artificial Sequence
  Synthetic
EVQLVESGGGLVQPGGSLRLSCAASGRTFVTYGMGWFRQAPGKGREFVSAISWSGSMTSYGDSVKGRFTISRDNAKNTLYLQMNSLRPEDTAVYYCAAALGAVVYTTREPYTYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 557 - protein, 356 aa, Artificial Sequence
  Synthetic
EVQLVESGGGLVQPGGSLRLSCAASGRTFVTYGMGWFRQAPGKGREFVSAISWSGSITSYGDSVKGRFTISRDNAKNTLYLQMNSLRPEDTAVYYCAAALGAVVYTTREPYTYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 558 - protein, 356 aa, Artificial Sequence
  Synthetic
EVQLVESGGGLVQPGGSLRLSCAASGRTFVTYGMGWFRQAPGKGREFVSAISWSGSLTSYGDSVKGRFTISRDNAKNTLYLQMNSLRPEDTAVYYCAAALGAVVYTTREPYTYWGQGTLVTVSSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 559 - protein, 580 aa, Artificial Sequence
  Synthetic
QVQLVQSGAEVKKPGASVKVSCKASGFNFEDTYMHWVRQAPGQGLEWMGRIDPADADTKYNPKFQDRVTITVDTSTNTAYMELSSLRSEDTAVYYCVRGNYVNWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVESGGGLVQPGGSLRLSCAASGRTFVTYGMGWFRQAPGKGREFVSAISWSGSMTSYGDSVKGRFTISRDNAKNTLYLQMNSLRPEDTAVYYCAAALGAVVYTTREPYTYWGQGTLVTVSS

SEQ ID NO: 560 - protein, 215 aa, Artificial Sequence
  Synthetic
EIVLTQSPGTLSLSPGERATLSCRASSSVSSSYLYWYQQKPGQAPRLLIYSTSNLASGIPDRFSGSGSGTDYTLTISRLEPEDAAVYFCHQWYSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 561 - protein, 584 aa, Artificial Sequence
  Synthetic
QVQLVQSGAEVKKPGSSVKVSCKASGYTFTSNYIHWVRQAPGQGLEWMGWIYPGDADTNYNQKFNGRVTLTADKSTSTAYMELSSLRSEDTAVYYCAINYGGIWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSEVQLVESGGGLVQPGGSLRLSCAASGRTFVTYGMGWFRQAPGKGREFVSAISWSGSMTSYGDSVKGRFTISRDNAKNTLYLQMNSLRPEDTAVYYCAAALGAVVYTTREPYTYWGQGTLVTVSS

SEQ ID NO: 562 - protein, 214 aa, Artificial Sequence
  Synthetic
DIQMTQSPSSLSASVGDRVTITCQASQDIGNKLIWYQQKPGKAPKLLIYYVTNLPGGVPSRFSGSGSGTDFTLTISSLQPEDFATYYCLQYKQNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 563 - protein, 580 aa, Artificial Sequence
  Synthetic
EVQLVESGGGLVQPGGSLRLSCAASGRTFVTYGMGWFRQAPGKGREFVSAISWSGSMTSYGDSVKGRFTISRDNAKNTLYLQMNSLRPEDTAVYYCAAALGAVVYTTREPYTYWGQGTLVTVSSGGGGSGGGGSGGGGSQVQLVQSGAEVKKPGASVKVSCKASGFNFEDTYMHWVRQAPGQGLEWMGRIDPADADTKYNPKFQDRVTITVDTSTNTAYMELSSLRSEDTAVYYCVRGNYVNWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 564 - protein, 584 aa, Artificial Sequence
  Synthetic
EVQLVESGGGLVQPGGSLRLSCAASGRTFVTYGMGWFRQAPGKGREFVSAISWSGSMTSYGDSVKGRFTISRDNAKNTLYLQMNSLRPEDTAVYYCAAALGAVVYTTREPYTYWGQGTLVTVSSGGGGSGGGGSGGGGSQVQLVQSGAEVKKPGSSVKVSCKASGYTFTSNYIHWVRQAPGQGLEWMGWIYPGDADTNYNQKFNGRVTLTADKSTSTAYMELSSLRSEDTAVYYCAINYGGIWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 565 - protein, 441 aa, Artificial Sequence
  Synthetic
QVQLVQSGAEVKKPGASVKVSCKASGFNFEDTYMHWVRQAPGQGLEWMGRIDPADADTKYNPKFQDRVTITVDTSTNTAYMELSSLRSEDTAVYYCVRGNYVNWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 566 - protein, 354 aa, Artificial Sequence
  Synthetic
EIVLTQSPGTLSLSPGERATLSCRASSSVSSSYLYWYQQKPGQAPRLLIYSTSNLASGIPDRFSGSGSGTDYTLTISRLEPEDAAVYFCHQWYSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGGSLRLSCAASGRTFVTYGMGWFRQAPGKGREFVSAISWSGSMTSYGDSVKGRFTISRDNAKNTLYLQMNSLRPEDTAVYYCAAALGAVVYTTREPYTYWGQGTLVTVSS

SEQ ID NO: 567 - protein, 445 aa, Artificial Sequence
  Synthetic
QVQLVQSGAEVKKPGSSVKVSCKASGYTFTSNYIHWVRQAPGQGLEWMGWIYPGDADTNYNQKFNGRVTLTADKSTSTAYMELSSLRSEDTAVYYCAINYGGIWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 568 - protein, 353 aa, Artificial Sequence
  Synthetic
DIQMTQSPSSLSASVGDRVTITCQASQDIGNKLIWYQQKPGKAPKLLIYYVTNLPGGVPSRFSGSGSGTDFTLTISSLQPEDFATYYCLQYKQNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSEVQLVESGGGLVQPGGSLRLSCAASGRTFVTYGMGWFRQAPGKGREFVSAISWSGSMTSYGDSVKGRFTISRDNAKNTLYLQMNSLRPEDTAVYYCAAALGAVVYTTREPYTYWGQGTLVTVSS

SEQ ID NO: 569 - protein, 354 aa, Artificial Sequence
  Synthetic
EVQLVESGGGLVQPGGSLRLSCAASGRTFVTYGMGWFRQAPGKGREFVSAISWSGSMTSYGDSVKGRFTISRDNAKNTLYLQMNSLRPEDTAVYYCAAALGAVVYTTREPYTYWGQGTLVTVSSGGGGSGGGGSGGGGSEIVLTQSPGTLSLSPGERATLSCRASSSVSSSYLYWYQQKPGQAPRLLIYSTSNLASGIPDRFSGSGSGTDYTLTISRLEPEDAAVYFCHQWYSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 570 - protein, 353 aa, Artificial Sequence
  Synthetic
EVQLVESGGGLVQPGGSLRLSCAASGRTFVTYGMGWFRQAPGKGREFVSAISWSGSMTSYGDSVKGRFTISRDNAKNTLYLQMNSLRPEDTAVYYCAAALGAVVYTTREPYTYWGQGTLVTVSSGGGGSGGGGSGGGGSDIQMTQSPSSLSASVGDRVTITCQASQDIGNKLIWYQQKPGKAPKLLIYYVTNLPGGVPSRFSGSGSGTDFTLTISSLQPEDFATYYCLQYKQNPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC